SEQUENCE LISTING (2 sequences)

SEQ ID NO: 1 - protein, 20 aa, Pyrrhocoris apterus
VDKGSYLPRPTPPRPIYNRN

SEQ ID NO: 2 - protein, 3 aa, Pyrrhocoris apterus
YLP

(19) United States
(12) Patent Application Publication
(10) Pub. No.: US 2010/0087466 A1
(43) Pub. Date: Apr. 8, 2010

(54) NOVEL SMALL MOLECULE DNAK INHIBITORS

(76) Inventors: Michael Alan Sturgess, Quakertown, PA (US); Frank Kotch, Monona, WI (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/553,648

(22) Filed: Sep. 3, 2009

Related U.S. Application Data
(60) Provisional application No. 61/093,881, filed on Sep. 3, 2008.
(60) Provisional application No. 61/186,658, filed on Jun. 12, 2009.

(51) Int. Cl. A61K 31/506; C07D 207/06; C07D 401/12; C07D 239/28; A61K 31/40; A61K 31/454; A61P 31/02; A61P 31/04

(52) U.S. Cl. 514/275; 548/533; 546/208; 544/332; 514/422; 514/326

(57) ABSTRACT

Methods of inhibiting HSP70 proteins, agents causing the inhibition of HSP70 proteins, and the effects of such inhibition on cell proliferation. Anti-microbial agents comprising small molecules, or pharmaceutical salts thereof, disclosed herein and further methods of use thereof are also disclosed. The disclosed small molecules, or pharmaceutical salts thereof, are effective in inhibiting microbial chaperone activity in microbes, such as homologs of HSP70. The disclosed small molecules, or pharmaceutical salts thereof, are also effective for the therapeutic treatment of cancer.

### CROSS REFERENCE TO RELATED APPLICATIONS

This application claims benefit of U.S. Provisional Application Ser. No. 61/093,881, filed Sep. 3, 2008, and U.S. Provisional Application Ser. No. 61/186,658, filed Jun. 12, 2009, under 35 U.S.C. §119(e), which are hereby incorporated by reference in their entirety.

### FIELD OF THE INVENTION

The present invention relates generally to methods of inhibiting HSP70 proteins, agents causing the inhibition of HSP70 proteins, and the effects of such inhibition on cell proliferation. More specifically, the invention relates to antimicrobial agents, and methods of use thereof wherein the antimicrobial agents comprise small molecules which bind to and inhibit bacterial chaperone proteins, such as DnaK and/or other bacterial homologs of HSP70. The invention also relates to methods and use of these small molecules as inhibitors of mammalian HSP70 proteins and the use of such species for the therapeutic treatment of cancer.

### BACKGROUND OF THE INVENTION

The continuing search for new and effective antibacterial agents which can treat infections caused by increasingly resistant microorganisms has identified a plethora of potential next generation agents. Many of these agents, however, have subsequently been shown to demonstrate either poor physiochemical properties, an increased tendency to induce bacterial resistance, poor toxicological profiles, or low efficiency in vivo. Accordingly, researchers are left to develop new approaches to combat bacterial infections within mammals.

One such approach is to target molecular mechanisms within the bacteria. For example, bacteria are known to respond to unfavorable conditions, such as elevated temperature. Elevated temperature is a human defense mechanism triggered by infection because it can damage proteins necessary for the survival of the bacteria. In response, many bacteria have evolved to develop molecular chaperones, such as bacterial homologs of human heat shock protein 70 (HSP70), designed to repair damaged proteins in order to survive the elevated temperature. These molecular chaperone proteins are essential because they not only repair proteins that become damaged or denatured as a consequence of unfavorable conditions, but, in doing so, activate signaling cascades that allow the bacteria to adapt to the conditions. One such bacterial HSP70 is DnaK.

DnaK is an acidic 70 kD molecular chaperone protein, often found in Gram negative bacteria. A bacterial homolog of mammalian Hsp70 protein, DnaK is the central protein in an ATP-driven multi-protein bacterial chaperone system, which includes other chaperone proteins DnaJ and GrpE. The DnaK chaperone system participates in a variety of cellular processes, including protein folding, protein translocation, and the assembly and disassembly of protein complexes. The DnaK chaperone system also regulates signal transduction pathways by controlling the stability and activities of transcriptional regulators and protein kinases. One role of this chaperone system is to catalyze the refolding of either unfolded or misfolded bacterial proteins. Another role of the DnaK chaperone system is the regulation of gene expression through the processing of specific RNA polymerase subunits. Thus, DnaK protects proteins that have been denatured by heat and peptides that are being synthesized. DnaK also blocks the folding of certain proteins that must remain unfolded until they have translocated across cellular membranes.

The structure of DnaK is comprised of a 638 amino acid sequence with an N-terminal nucleotide binding domain, containing an ATPase active site, and a C-terminal substrate binding domain. The substrate binding domain is further subdivided into two parts, a beta-sandwich subdomain and an alpha-helical subdomain. The beta-sandwich subdomain holds the substrate binding groove, while the alpha-helical subdomain functions as a lid over the substrate binding groove and is thought to control the accessibility to the substrate binding site. Specifically, the alpha-helical subdomain is comprised of five (5) alpha helices, A-E. Alpha helices A and B are packed onto the beta-sandwich subdomain. Helix B, together with helices C, D and E, build up a hydrophobic helical core that acts as a lid over the peptide binding groove of the beta-sandwich subdomain.

DnaK's substrate binding domain binds to regions of unfolded proteins that are rich in hydrophobic residues, thus preventing the breakdown and/or facilitating in the folding of such proteins. DnaK binds to and releases proteins in a cycle that involves ATP hydrolysis and several other chaperone proteins, including DnaJ and GrpE. In an ATP-dependent reaction cycle, DnaK binds to a stretch of exposed hydrophobic residues on partially denatured protein and refolds the protein in concert with DnaK-mediated ATP hydrolysis. It is this mechanism or similar mechanism with other HSP70 homologs, that many bacteria utilize to overcome heat exposure and protein denaturation as a result of the increase temperature or immune response within mammals.

A subset of the insect-derived antibacterial peptides has been found to inhibit the chaperone activity of bacterial homologs of HSP70, such as of DnaK. One such peptide is pyrrhocoricin, a 20 amino acid residue peptide from Pyrrhocoris (Cociancich et al., Biochem. J. 300:567-575 (1994)). Pyrrhocoricin is a glycopeptide with the following amino acid sequence: Val-Asp-Lys-Gly-Ser-Tyr-Leu-Pro-Arg-Pro-Thr-Pro-Pro-Arg-Pro-Ile-Tyr-Asn-Arg-Asn (SEQ ID NO: 1). This peptide is rich in the amino acid proline and is glycosylated on its Thr-11 amino acid residue with N-acetylgalactosamine, a disaccharide. Pyrrhocoricin is hydrophilic, which is evident because 50% of its amino acid residues are charged and/or polar while only three of its amino acid residues are hydrophobic. While the natural peptide is glycosylated at Thr-11, the non-glycosylated peptide has been shown to be similarly active, indeed most studies with this peptide have employed the non-glycosylated C-terminal amide analog as a surrogate of pyrrhocoricin.

The interaction of pyrrhocoricin with bacterial homologs of HSP70, e.g. DnaK, has been studied to some extent (see, e.g., Otvos, Jr. et al., Biochemistry 39:14150-14159 (2000); Kragol et al., Biochemistry 40:3016-3026 (2001); Liudmila et al., FEBS Lett. 565: 65-69 (2004)). The mode of action of this peptide is inhibition of DnaK, and thus, inhibition of protein folding. Specifically, residues within the N-terminal of the peptide, Val 1-Pro 10, have been implicated in binding to DnaK, and were shown to interact with an unnatural peptide construct mimicking the helices D and E of the helical lid of the bacterial protein (Kragol et al., Eur. J. Biochem. 269:4226-4237 (2002)). However, once bound to the DnaK, the precise mechanism through which pyrrhocoricin inhibits DnaK activity is in dispute. Some researchers report that pyrrhocoricin works by acting as an inhibitor of the ATPase activity of DnaK and that this inhibition prevents DnaK from assisting the folding of damaged proteins necessary to maintain the life of bacteria (Otvos, Jr. et al., Biochemistry 39:14150-14159 (2000)). Other researchers report that pyrrhocoricin actually stimulates the ATPase activity of DnaK and binds to the peptide binding groove of DnaK, which would normally encompass unfolded protein sequences. It is this competition that reportedly inhibits DnaK. (Liudmila et al., FEBS Lett. 565: 65-69 (2004)). Nonetheless, it is clear that Pyrrhocoricin is known to inhibit DnaK's chaperone activity. In analogous studies, however, pyrrhocoricin was shown not to interact with the same region of DnaK from Gram-positive bacteria (e.g. S. aureus). In either case, pyrrhocoricin's inhibitory activity was accomplished without disrupting bacterial cellular membranes.

In the presence of mammalian serum, however, pyrrhocoricin is known to be highly susceptible to degradation. When pyrrhocoricin degrades, it becomes inactive as an antibacterial agent. Specifically, it was previously shown that pyrrhocoricin, when exposed to mammalian serum in a naked form, enzymatically degrades to structurally inactive proteolytic cleavage products. To this end, administration of pyrrhocoricin to a human as an antimicrobial agent is ineffective because the peptide is inactivated within the human body before it may be absorbed within the bacterial invader.

Accordingly, there is a continuing need for novel anti-bacterial agents that inhibit chaperone protein activity, such as bacterial homologs of HSP70, of bacterial invaders of the human body. More specifically, there is a continuing need for novel anti-bacterial agents that inhibit the activity of HSP70 bacterial homologs, while retaining activity as an antibacterial agent upon administration into the human body. The present invention, as described herein, addresses these needs.

### SUMMARY OF THE INVENTION

The present invention fills the foregoing needs by providing novel small molecules, or pharmaceutical salts thereof, for inhibiting the activity of DnaK, bacterial HSP70 or any homolog thereof. As noted above, the polypeptide pyrrhocoricin acts as an antibacterial agent through its inhibition of bacterial chaperone proteins such as DnaK. Yet pyrrhocoricin becomes highly susceptible to degradation when introduced into the human body. In addition, this peptide demonstrated an unacceptable toxicity profile in animals, possibly associated with the tendency of the peptide to disrupt cellular membranes at high concentrations. Furthermore, the size and highly hydrophilic nature of these peptides, and the cost of producing these peptides at production scale renders them less than ideal antibacterial agents. Accordingly, the present invention reduces the essential chaperone binding motif of pyrrhocoricin to optimize the antibacterial effect on HSP70 bacterial homolog proteins while reducing the susceptibility to degradation and potential for membrane damage when introduced into the human body. To this end, applicants have discovered that the administration of the present invention inhibits bacterial chaperone proteins and, ultimately, is effective as an anti-microbial agent.

Applicants have also discovered the use of these small molecules as inhibitors of mammalian HSP70 proteins and the use of such species for the therapeutic treatment of cancer.

A specific embodiment of the invention includes a small molecule or polypeptide, or a pharmaceutical salt thereof, defined by the structure of Formula I:

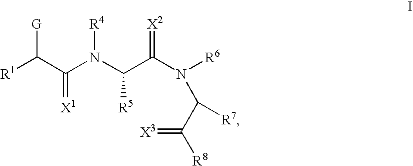

wherein X¹X², and X³ are substituent groups independently selected from the group consisting of an oxygen atom, a sulfur atom, and two hydrogen atoms.

R¹ is a substituent group selected from the group consisting of a hydrogen, 1 to 16 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 10 carbon hydroxyalkyl group, a 1 to 6 carbon alkylamino group, a 1 to 6 carbon dialkylamino group, a 1 to 6 carbon aminoalkyl group, a 1 to 6 carbon alkoxyamino group, or a substituent group defined by Formula II(a):

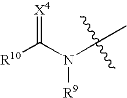

wherein R⁹ is a substituent group selected from the group consisting of a hydrogen atom and a 1 to 6 carbon alkyl; R¹⁰ is a substituent group selected from the group consisting of a hydroxyl group, a hydroxyalkyl, a 1 to 17 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 6 carbon alkylamino group and a 1 to 6 carbon dialkylamino group, and an aminoalkyl group; and X⁴ is a substituent group selected from the group consisting of an oxygen atom, a sulfur atom, and two hydrogen atoms.

G is a substitutent group selected from the group consisting of a benzyl group, a substituted benzyl group, a phenyl group, a substituted phenyl group, a heteroarylalkyl group wherein a hydrogen atom of an alkyl group is substituted by a heteroaryl group, a 1 to 6 carbon alkyl group, or a substituted 1 to 6 carbon alkyl group. In one embodiment, the substituted phenyl group and substituted benzyl groups are defined by the respective formulas:

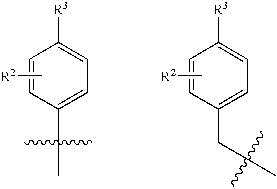

wherein R² is a substituent group selected from a group consisting of a hydrogen atom, a 1 to 6 carbon alkyl group, a hydroxyl group, a 1 to 2 ring aralkyloxy group, a 1 to 10 carbon alkoxy group, a halo group, an amine group, a 1 to 6 carbon alkylamino group, a carboxylic acid ester, an amide group, or a 1 to 6 carbon dialkylamino group. In a further embodiment, the substituent group of R² is capable of forming a cyclic unit in conjunction with R³ or R⁴.

R³ is a substituent group which is selected from the group consisting of a hydrogen atom, a hydroxyl group, a 1 to 6 carbon alkyl group, a 1 to 10 carbon alkoxy group, a 1 to 6 carbon hydroxyalkyl group, a 1 to 2 ring aralkyloxy group, a 1 to 2 ring aryloxy group, a halo group, a sulfidryl group, an alkylsulfidryl group, an amine group, a 1 to 6 carbon alkylamino group, a carboxylic acid ester, an amide group, and a 1 to 6 carbon dialkylamino group, a carboxylic acid ester, an amide group, and a substituent group represented by the structure of Formula II(b):

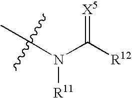

wherein X⁵ is a substituent group selected from the group consisting of an oxygen atom, a sulfur atom, and two hydrogen atoms; R¹¹ is a substituent group selected from the group consisting of a hydrogen atom and a 1 to 6 carbon alkyl group; and R¹² is a substituent group selected from the group consisting of a hydroxyl group, a 1 to 17 carbon alkyl group, a 1 to 10 carbon alkoxy group, a 1 to 2 ring aralkyloxy group, a 1 to 2 ring aryloxy group, a 1 to 6 carbon alkylamino group, and a 1 to 2 ring aralkylamino. In one embodiment, the R³ substituent group is capable of forming a cyclic ring structure in conjunction with an R² or an R⁴ substituent group.

R⁴ is a substituent group selected from the group consisting of a hydrogen atom and a 1 to 6 carbon alkyl group wherein the R⁴ substituent group is capable of forming a cyclic ring structure in conjunction with a G, an R³ or an R² substituent group.

R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group.

R⁶ is a substituent group selected from the group consisting of a hydrogen atom and a 1 to 6 carbon alkyl group optionally substituted wherein the R⁶ substituent group is capable of forming a cyclic ring structure in conjunction with an R⁷ substituent group.

R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group.

R⁸ is a substituent group which is selected from the group consisting of a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a secondary amine, a tertiary amine, a tertiary cyclic amine optionally substituted by a guanidinylalkyl group, a 1 to n carbon aminoalkylamino group where n=2-6 and derivatives thereof such as an amide, a carbamate, a urea, an amidine and a guanidine, an aminoalkyl group and derivatives thereof such as an amide, a carbamate, a urea, an amidine and a guanidine. Aminoalkylamino R⁸ groups are represented generally by the formula NH(CH₂)ₙNH₂, with the aforementioned derivatives represented by formulas such as NH(CH₂)ₙNHC(O)Me, NH(CH₂)ₙNHC(═NH)NH₂, and the like. It is contemplated that R⁸ includes compounds with an additional alkyl substituent within the (CH₂)ₙ chain, which can be either cyclic or acyclic. In at least one embodiment, the R⁸ substituent comprises a peptide moiety having 1-4 amino acids bound at the R⁸ position by an N-terminus end of the peptide moiety. Additional substituent groups may extend from an N terminus, a side chain and/or a C terminus of each amino acid within the peptide moiety wherein the additional substituent groups may be selected from the group consisting of a hydrogen atom, a hydroxyl group, a heterocyclic aromatic compound, an alkyl ester group, a cycloalkyl group, an amino alkyl group, a diaminoalkyl group, a primary amine, a secondary amine, and a tertiary amine.

As noted above, the G substituent group is comprised of a benzyl group, a substituted benzyl group, a heterocyclic aromatic alkyl group, a phenyl group, a substituted phenyl group, an 1 to 6 carbon alkyl group, or a substituted 1 to 6 carbon alkyl group. To this end, in a preferred, but non-limiting, embodiment the G substituent group is a benzyl group and the structure of the present invention is defined by Formula III:

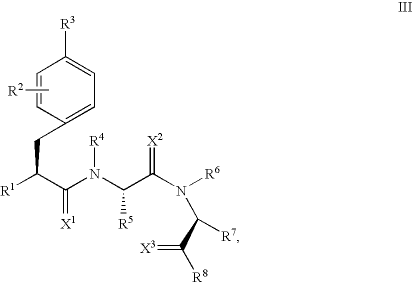

wherein X¹ is a substituent group selected from the group consisting of an oxygen atom, a sulfur atom, and two hydrogen atoms.

X² is a substituent group selected from the group consisting of an oxygen atom, a sulfur atom, and two hydrogen atoms.

X³ is a substituent group selected from the group consisting of an oxygen atom, a sulfur atom, and two hydrogen atoms.

R¹ is a substituent group selected from the group consisting of a hydrogen, 1 to 16 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 10 carbon hydroxyalkyl group, a 1 to 6 carbon alkylamino group, a 1 to 6 carbon dialkylamino group, a 1 to 6 carbon aminoalkyl group, a 1 to 6 carbon alkoxyamino group, or a substituent group defined by Formula II(a):

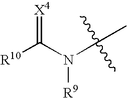

wherein R⁹ is a substituent group selected from the group consisting of a hydrogen atom and a 1 to 6 carbon alkyl; R¹⁰ is a substituent group selected from the group consisting of a hydroxyl group, a 1 to 17 carbon alkyl group, a 3 to 6 carbon cycloalkyl group, a 1 to 2 ring aralkyl group, a 1 to 2 ring aryl group, a 1 to 10 carbon alkoxy group, a 1 to 6 carbon alkylamino group and a 1 to 6 carbon dialkylamino group; and X⁴ is a substituent group selected from the group consisting of an oxygen atom, a sulfur atom, and two hydrogen atoms.

R² is a substituent group selected from a group consisting of a hydrogen atom, a 1 to 6 carbon alkyl group, a hydroxyl group, a 1 to 2 ring aralkyloxy group, a 1 to 10 carbon alkoxy group, a halo group, an amine group, a 1 to 6 carbon alkylamino group, a carboxylic acid ester, an amide group, or a 1 to 6 carbon dialkylamino group. In another embodiment, the substituent group of R² is capable of forming a cyclic unit in conjunction with R³ or R⁴.

R³ is a substituent group which is selected from the group consisting of a hydrogen atom, a hydroxyl group, a 1 to 6 carbon alkyl group, a 1 to 10 carbon alkoxy group, a 1 to 6 carbon hydroxyalkyl group, a 1 to 2 ring aralkyloxy group, a 1 to 2 ring aryloxy group, a halo group, a sulfidryl group, an alkylsulfidryl group, an amine group, a 1 to 6 carbon alkylamino group, a carboxylic acid ester, an amide group, and a 1 to 6 carbon dialkylamino group, a carboxylic acid ester, an amide group, and a substituent group comprising Formula II(b):

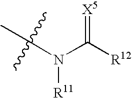

wherein X⁵ is a substituent group selected from the group consisting of an oxygen atom, a sulfur atom, and two hydrogen atoms; R¹¹ is a substituent group selected from the group consisting of a hydrogen atom and a 1 to 6 carbon alkyl group; and R¹² is a substituent group selected from the group consisting of a hydroxyl group, a 1 to 17 carbon alkyl group, a 1 to 10 carbon alkoxy group, a 1 to 2 ring aralkyloxy group, a 1 to 2 ring aryloxy group, a 1 to 6 carbon alkylamino group, and a 1 to 2 ring aralkylamino. In one embodiment, the R³ substituent group is capable of forming a cyclic ring structure in conjunction with an R² or an R⁴ substituent group.

R⁴ is a substituent group selected from the group consisting of a hydrogen atom and a 1 to 6 carbon alkyl group wherein the R⁴ substituent group may be able to form a cyclic ring structure in conjunction with a G, an R³ or an R² substituent group.

R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group.

R⁶ is a substituent group selected from the group consisting of a hydrogen atom and a 1 to 6 carbon alkyl group wherein the R⁶ substituent group is capable of forming a cyclic ring structure in conjunction with an R⁷ substituent group.

R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group.

R⁸ is a substituent group which is selected from the group consisting of a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a secondary amine, a tertiary amine, a tertiary cyclic amine optionally substituted by a guanidinylalkyl group, a 1 to n carbon aminoalkylamino group where n=2-6 and derivatives thereof such as an amide, a carbamate, a urea, an amidine and a guanidine, an aminoalkyl group and derivatives thereof such as an amide, a carbamate, a urea, an amidine and a guanidine. Aminoalkylamino R⁸ groups are represented generally by the formula NH(CH₂)ₙNH₂, with the aforementioned derivatives represented by formulas such as NH(CH₂)ₙNHC(O)Me, NH(CH₂)ₙNHC(═NH)NH₂, and the like. It is contemplated that R⁸ includes compounds with an additional methyl substituent within the (CH₂)ₙ chain. In at least one embodiment, the R⁸ substituent comprises a peptide moiety having 1-4 amino acids bound at the R⁸ position by an N-terminus end of the peptide moiety. Additional substituent groups may extend from an N terminus, a side chain and/or a C terminus of each amino acid within the peptide moiety wherein the additional substituent groups may be selected from the group consisting of a hydrogen atom, a hydroxyl group, a heterocyclic aromatic compound, an alkyl ester group, a cycloalkyl group, an amino alkyl group, a diaminoalkyl group, a primary amine, a secondary amine, and a tertiary amine.

As noted above, the small molecules, or pharmaceutical salts thereof, of the present invention are effective in inhibiting bacterial chaperone activity. More specifically, the present invention is effective in inhibiting the activity of bacterial homologs of HSP70, e.g. DnaK. Substituent groups are added to the core structure of the present invention, embodied within Formulas I and III, so as to prevent the core peptide structure from degrading during administration and/or to improve the interaction with the DnaK protein. To this end, in one embodiment, one or more small molecules, or pharmaceutical salts thereof, of the above structures may be administered as an antimicrobial agent to a mammal infected with a bacteria. The small molecules, or pharmaceutical salts thereof, may be administered as an anti-bacterial composition within a pharmaceutically acceptable carrier or vehicle suitable for administration as a protein composition.

The pharmaceutical compositions of the present invention may also be formulated to suit a selected route of administration, and may contain ingredients specific to the route of administration. To this end, any route of administration may be utilized so long as the small molecule, or a pharmaceutical salt thereof, of the present invention is placed into contact with the targeted bacteria, such as by way of the blood stream of the mammal. Exemplary examples of administration include, but are not limited to, injection (e.g. intravenous intradermal, intramuscular, or subcutaneous) or oral (e.g. a pill, tablet capsule, solution, syrup, elixir, suspension, gel, or powder). The formulation may also include diluents as well as, in some cases, adjuvants, buffers, preservatives and the like.

The dosage of the small molecules, or pharmaceutical salts thereof, of the present invention in each anti-bacterial effective dose is selected with regard to consideration of the pathogen causing the infection, the severity of infection, the patient's age, weight, sex, general physical condition and the like. The small molecules, or pharmaceutical salts thereof, of the present invention, or compositions thereof, will generally be used in an amount effective to achieve the intended purpose. To this end, dosages of the small molecules, or pharmaceutical salts thereof, will depend on the particular application. For example, for use to treat or prevent microbial infections or diseases related thereto, the small molecules of the present invention, or pharmaceutical salts thereof, are administered or applied in a therapeutically effective amount. Therapeutically effective amount means an amount effective to ameliorate the symptoms of, or ameliorate, treat or prevent microbial infections or diseases related thereto. Determination of a therapeutically effective amount is well within the capabilities of those skilled in the art, especially in light of the detailed disclosure and examples provided herein.

The small molecules, or pharmaceutical salts thereof, of the present invention may be administered along with or in combination with other active agents, such as conventional antibiotics or any other anti-microbial agent known in the art. To this end, coadministration of such agents enhances the antibacterial potential of the small molecules and the antibacterial agent. Such synergistic interaction of antibacterial agents has been demonstrated in the case of pyrrhocoricin, and analogs of pyrrhocoricin, when administered in combination with fluoroquinolones and aminoglycosides. Such coadministration to patients suffering from a bacterial infection has the possible benefit of i) lowering the necessary therapeutically effective dose; ii) extending the duration of activity of a fixed dose; iii) reducing the likelihood of the development of resistant strains of the infecting organism; and/or iv) expanding the spectrum of activity of the individual agents. Agents that exhibit a therapeutic synergistic effect when coadministered are preferred.

### DETAILED DESCRIPTION OF THE INVENTION

The present invention includes novel small molecules exhibiting anti-bacterial activity by inhibiting bacterial chaperone protein activity. The small molecules are structurally based on the naturally occurring glycosylated peptide, pyrrhocoricin, yet are modified to reduce the essential bacterial chaperone binding motif to a substituted polypeptide and to prevent degradation within human serum. Specifically, amino acid residues Val-1 through Pro-10 of SEQ ID NO: 1 are known to comprise the essential chaperone binding motif of pyrrhocoricin. However, administration of this 10 amino acid peptide as an anti-microbial agent is ineffective. The present invention overcomes this problem by administering variants of the pyrrhocoricin based peptide sequence Tyr-Leu-Pro (SEQ ID NO: 2) (amino acids 6-8 of the pyrrhocoricin) as a minimal unit to create a structure which optimally binds to bacterial chaperone proteins without being highly susceptible to degradation when introduced into mammalian serum. To aid in the understanding of the present invention, the following non-limiting definitions are provided:

The term “alkyl” refers to a group of atoms derived from a straight or branched-chain hydrocarbon molecule by removing a hydrogen from one of the carbon atoms. Alkyl groups and the alkyl portions of other groups defined herein may be alkyl-substituted by alkyl groups of one to four carbon atoms. Methyl substituents are preferred.

The term “alkoxy group” refers to an alkyl group linked to an oxygen atom. In other words, an “alkoxy group” is a group derived from an alkyl alcohol molecule by removing the hydrogen from the hydroxyl group.

The term “alkylamino group” refers to a group that consists of a nitrogen atom attached by a single bond to a group of carbon and hydrogen atoms arranged in an alkyl chain. In other words, an “alkylamino group” includes a group derived from an amine molecule by replacing one or more hydrogen atoms with alkyl groups. The alkyl portion of the amino group comprises a carbon chain, as exemplified in the preferred embodiments, and is attached to a molecular backbone via a nitrogen atom of said amino group.

The term “alkylsulfidryl group” refers to a group that consists of an alkyl group connected to a sulfidryl group.

The term “amide” refers to a carbonyl group linked to an amine group forming R′₁C(O)NR′₂R′₃, wherein the R′₁, R′₂ and R′₃ can be any atom or functional group deemed by those of ordinary skill in the art suitable. For example, the amine group may be a primary, secondary or tertiary amine and each R′₁, R′₂ and R′₃ is independently any suitable substituent group discussed herein. The term “amide” is used herein regardless of its point of attachment to a core structure. Further, the term “amide” used herein includes functional groups such as carbamates and ureas. The term “carbamate” is directed to amides that share a common functional group with the general structure —NR′₁(CO)O—R′₂. The term “Urea” is directed to amides that share a common functional group with the general formula —NR′₁(CO)NR′₂—. R′₁ and R′₂ are as described above. The term “amine” refers to an organic substituent group composed of a nitrogen atom, N.

The term “amino acid” refers to alpha amino acids, molecules that contains both amine and carboxyl substituent groups attached to the same carbon, which is called the alpha carbon. Amino acids are the basic building blocks of proteins. They connect to form short polymer chains called peptides, or longer polymer chains called polypeptides or proteins.

The term “aminoalkyl group” refers to an alkyl group substituted by at least one amino group in place of its hydrogen atom, wherein the group is attached to a molecular backbone via a carbon atom of said alkyl group. In other words, an “aminoalkyl group” includes a group derived from an amine molecule by replacing one or more hydrogen atoms with alkyl groups and is attached to a molecular core directly via a carbon atom of its alkyl chain.

The term “aminoalkylamino” refers to an alkylamino group having one of the hydrogen atoms of the alkyl group replaced by a nitrogen atom.

The term “aralkyloxy group” refers to an alkoxy group having one of its hydrogen atoms on the alkyl group replaced by an aryl group.

The term “aryl group” refers to a group of atoms derived from a molecule containing aromatic ring(s) by removing one hydrogen that is bonded to the aromatic ring(s).

The term “aryloxy group” refers to a group derived from a molecule containing aromatic ring(s) by replacing one hydrogen on the aromatic ring(s) with an oxygen atom.

The term “aralkyl group” refers to a group in which an aryl group is substituted for a hydrogen atom of an alkyl group.

The term “benzyl” refers to a group of atoms derived from a benzene ring wherein one hydrogen is substituted with a methylene group, as represented by PhCH₂—.

The term “Boc” or t-Boc, stands for tert-Butyloxy-carbonyl.

The term “cycloalkyl” refers to a group of atoms derived from a non-aromatic monocyclic or polycyclic molecule comprised of carbon and hydrogen atoms by removing one hydrogen bonded to the carbon ring(s).

The term “carboxylic acid” refers to an organic acid characterized by the presence of a carboxyl group which has the formula —COOH.

The term “carboxylic acid ester” refers to a carboxylic acid group (C(O)OH) with any of the substituent groups described herein replacing the hydrogen atom of the hydroxyl group forming (C(O)OR′₁).

The term “dialkylamino group” refers to a group derived from an amine molecule by replacing two hydrogen atoms with two alkyl groups such that each hydrogen can be replaced by an independent 1 to 6 carbon alkyl group.

The term “Fmoc” stands for Fluorenyl-methoxy-carbonyl.

The terms “guanidine” and “guanidines” are directed to a group of organic compounds that share a common functional group with the general formula:

(R′₁R′₂N)—(C═N—R′₃)—R′₁R′₂N

Thus these terms include substituted guanidines, including imide-substituted guanidines. The term “guanidinium” refers to a protonated guanidine with a positive charge. The term “guanidinyl” is directed to a functional group having the structure (R₁R₂N)—C(═N—R₃)—N(R₁′)—, wherein R₁, R₂, R₁′, and R₃ are each independently hydrogen or C₁-C₄ alkyl.

The term “guanidinylalkyl” refers to a C₁-C₄ alkyl group substituted by a guanidinyl group, for example, formula —(CH₂)ₙ—NHC(═NH)NH₂, wherein n is an integer selected from 1 through 4, represents a few simple “guanidinylalkyl” groups.

The term “halo” refers to the halogens, a series of non-metal elements from group 7 of the periodic table. The halogen elements are comprised of fluorine, F, chlorine, Cl, bromine, Br, iodine, I, and astatine, At.

The term “heterocyclic aromatic compound” refers to an aromatic ring, e.g. benzene, wherein one or more of the carbon atoms of the ring have been replaced by a heteroatom. By the term “heteroatom” it is meant any atom that is not carbon or hydrogen, typically, nitrogen, oxygen, and sulfur.

The term “heterocyclic aromatic alkyl” refers to an alkyl group in which a hydrogen atom is substituted by a heterocyclic aromatic group as defined herein.

The term “hydroxyl” refers to a group consisting of an oxygen atom and a hydrogen atom connected by a covalent bond. The term “hydroxyl group” is used to describe the substituent group —OH when it is substituted in place of a hydrogen atom on an organic molecule.

The term “hydroxyalkyl group” refers to an alkyl group having one of its hydrogen atoms replaced by a hydroxyl group.

The term “primary amine” refers to an amine group with two hydrogens and one non-hydrogen organic substituent group.

The term “secondary amine” refers to an amine group with one hydrogen and two non-hydrogen organic substituent groups.

The term “sulfidryl” refers to a thiol, a compound that contains the substituent group composed of a sulfur atom and a hydrogen atom, —SH.

The term “tertiary amine” refers to an amine group with three non-hydrogen organic substituent groups.

The term “tertiary cyclic amine” refers to an amino group with the amino nitrogen (N) belonging to a 5- or 6-membered non-aromatic ring structure. Representative examples of “tertiary cyclic amine” include, but not limited to, piperidinyl, piperazinyl, and pyrrolidinyl.

In one embodiment of the present invention, anti-bacterial small molecules are based upon the reduction of the bacterial chaperone binding motif of pyrrhocoricin Tyr-Leu-Pro (SEQ ID NO: 2) (amino acids 6-8) by various substituent groups. To this end, the antibacterial small molecules and/or peptides of the present invention are defined by Formula I:

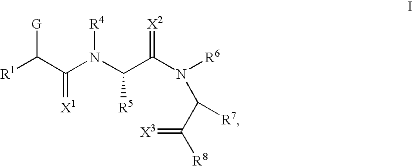

wherein X¹ is a substituent group selected from the group consisting of an oxygen atom, a sulfur atom, and two hydrogen atoms.

X² is a substituent group selected from the group consisting of an oxygen atom, a sulfur atom, and two hydrogen atoms.

X³ is a substituent group selected from the group consisting of an oxygen atom, a sulfur atom, and two hydrogen atoms.

R¹ is a substituent group selected from the group consisting of a hydrogen, 1 to 16 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 10 carbon hydroxyalkyl group, a 1 to 6 carbon alkylamino group, a 1 to 6 carbon dialkylamino group, a 1 to 6 carbon aminoalkyl group, a 1 to 6 carbon alkoxyamino group, or a substituent group defined by Formula II(a):

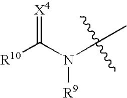

wherein R⁹ is a substituent group selected from the group consisting of a hydrogen atom and a 1 to 6 carbon alkyl; R¹⁰ is a substituent group selected from the group consisting of a hydroxyl group, a 1 to 17 carbon alkyl group, a 3 to 6 carbon cycloalkyl group, a 1 to 2 ring aralkyl group, a 1 to 2 ring aryl group, a 1 to 10 carbon alkoxy group, a 1 to 6 carbon alkylamino group and a 1 to 6 carbon dialkylamino group; and X⁴ is a substituent group selected from the group consisting of an oxygen atom, a sulfur atom, and two hydrogen atoms.

G is a substitutent group selected from the group consisting of a benzyl group, a substituted benzyl group, a phenyl group, a substituted phenyl group, a heteroarylalkyl group wherein a hydrogen atom of an alkyl group is substituted by a heteroaryl group, a 1 to 6 carbon alkyl group, or a substituted 1 to 6 carbon alkyl group. In one embodiment, the substituted phenyl group and substituted benzyl groups are defined by the respective formulas:

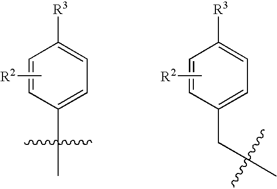

wherein R² is a substituent group selected from a group consisting of a hydrogen atom, a 1 to 6 carbon alkyl group, a hydroxyl group, a 1 to 2 ring aralkyloxy group, a 1 to 10 carbon alkoxy group, a 1 to 10 carbon hydroxyalkyl, a halo group, an amine group, a 1 to 6 carbon alkylamino group, a carboxylic acid ester, an amide group, an aminoalkyl or a 1 to 6 carbon dialkylamino group. In one embodiment, the substituent group of R² is capable of forming a cyclic unit in conjunction with R³ or R⁴.

R³ is a substituent group which is selected from the group consisting of a hydrogen atom, a hydroxyl group, a 1 to 6 carbon alkyl group, a 1 to 10 carbon alkoxy group, a 1 to 6 carbon hydroxyalkyl group, a 1 to 2 ring aralkyloxy group, a 1 to 2 ring aryloxy group, a halo group, a sulfidryl group, an alkylsulfidryl group, an amine group, a 1 to 6 carbon alkylamino group, a carboxylic acid ester, an amide group, an aminoalkyl group, a 1 to 6 carbon dialkylamino group, a carboxylic acid ester, and a substituent group represented by the structure of Formula II(b):

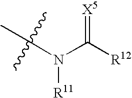

wherein X⁵ is a substituent group selected from the group consisting of an oxygen atom, a sulfur atom, and two hydrogen atoms; R¹¹ is a substituent group selected from the group consisting of a hydrogen atom and a 1 to 6 carbon alkyl group; and R¹² is a substituent group selected from the group consisting of a hydroxyl group, a 1 to 17 carbon alkyl group, a 1 to 10 carbon alkoxy group, a 1 to 2 ring aralkyloxy group, a 1 to 2 ring aryloxy group, a 1 to 6 carbon alkylamino group, and a 1 to 2 ring aralkylamino. In one embodiment, the R³ substituent group is capable of forming a cyclic ring structure in conjunction with an R² or an R⁴ substituent group.

In another embodiment, as discussed above, the G substituent group may be comprised of a 1 to 6 carbon substituted alkyl group. To this end, the hydrogen atoms at any point along the alkyl group may be substituted with one or more heteroatoms, additional alkyl groups, cycloalkyl groups, aryl groups, aralkyl groups, alkoxy groups, amine groups, amide groups, aromatic rings structures, heteroaromatic ring structures, aromatic analogs of a known natural or unnatural amino acid, or heteroaromatic analogs of a known natural or unnatural amino acid. In a non-limiting example, the alkyl group is comprised of a methyl group wherein at least one hydrogen atom is replaced with a substitutent group other than a hydrogen. In one embodiment, the substitutent group is a thienyl group wherein the carbon atom adjacent/attached to the sulfur atom of the thienyl group is bound to the methyl carbon. As such, in this embodiment the G substituent group forms a 3-(2-thieny)-alanine. In another embodiment, the substituent group is a benzothienyl group wherein the carbon atom adjacent/attached to the sulfur atom of the thienyl group is bound to the methyl carbon. As such, in this embodiment the G substituent group form a 3-(2-benzothieny)-alanine. However, the present invention is not limited to these embodiments and may include any other aromatic or heteroaromatic compounds, such as five to six member aromatic or heteroaromatic rings, or any other substituent group discussed herein.

R⁴ is a substituent group selected from the group consisting of a hydrogen atom and a 1 to 6 carbon alkyl group wherein the R⁴ substituent group may be able to form a cyclic ring structure in conjunction with a G, an R³ or an R² substituent group.

R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group

R⁶ is a substituent group selected from the group consisting of a hydrogen atom and a 1 to 6 carbon alkyl group wherein the R⁶ substituent group is capable of forming a cyclic ring structure in conjunction with an R⁷ substituent group.

R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group.

R⁸ is a substituent group which is selected from the group consisting of a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a secondary amine, a tertiary amine, a tertiary cyclic amine optionally substituted by a guanidinylalkyl group, a 1 to n carbon aminoalkylamino group where n=2-6 and derivatives thereof such as an amide, a carbamate, a urea, an amidine and a guanidine, an aminoalkyl group and derivatives thereof such as an amide, a carbamate, a urea, an amidine and a guanidine. Aminoalkylamino R⁸ groups are represented generally by the formula NH(CH₂)ₙNH₂, with the aforementioned derivatives represented by formulas such as NH(CH₂)ₙNHC(O)Me, NH(CH₂)ₙNHC(═NH)NH₂, and the like. In at least one embodiment, the R⁸ substituent comprises a peptide moiety having 1-4 amino acids bound at the R⁸ position by an N-terminus end of the peptide moiety. Additional substituent groups may extend from an N terminus, a side chain and/or a C terminus of each amino acid within the peptide moiety wherein the additional substituent groups may be selected from the group consisting of a hydrogen atom, a hydroxyl group, a heterocyclic aromatic compound, an alkyl ester group, a cycloalkyl group, an amino alkyl group, an aminoalkylamino group, a primary amine, a secondary amine, and a tertiary amine.

Referring to Formula I, in one embodiment, the G substitutent group is a 4 carbon butyl group. The butyl group may be a straight-chain alkyl group or a branched chain such as a sec-butyl group (as illustrated in Example 22).

In another embodiment of Formula I, the G substituent group is a substituted benzyl group. To this end, the compounds of present invention may be defined by Formula III:

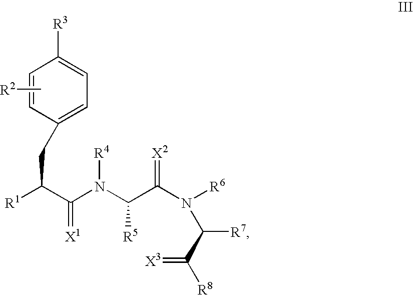

Wherein X¹, X², and X³ can be an oxygen atom, a sulfur atom, and two hydrogen atoms.

R¹ is a substituent group selected from one of the following groups: (a) an alkyl group; (b) a cycloalkyl group; (c) an aralkyl group; (d) an aryl group; (e) an alkoxy group; (f) an alkylamino group; (g) a dialkylamino group; (h) a hydrogen; (j) an alkoxyamino group; (k) a hydroxyalkyl group, (l) an amide group, (m) an aminoalkyl group, (m) a primary amine; (n) a secondary amine; or (o) a tertiary amine. More specifically, the R¹ substituent group is a hydrogen, any 1 to 6 carbon alkyl group, a 3 to 6 carbon cycloalkyl group, a 1 to 2 ring aralkyl group, a 1 to 2 ring aryl group, a 1 to 10 carbon alkoxy group, a 1 to 6 carbon alkylamino group, a 1 to 6 carbon dialkylamino group, or a substituent group comprising Formula II(a):

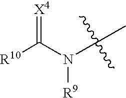

In a further embodiment, the small molecule, or pharmaceutical salts thereof, of the present invention are directed to a combination of Formula II and Formula III yielding Formula III(A):

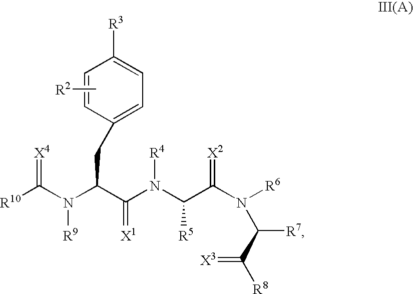

wherein R⁹ is a substituent group which is selected from one of the following: (a) a hydrogen atom; or (b) an alkyl group, such as a 1 to 6 carbon alkyl group. R¹⁰ is a substituent group selected from one of the following groups: (a) an alkyl group; (b) a cycloalkyl group; (c) an aralkyl group; (d) an aryl group; (e) an alkoxy group; (f) an alkylamino group; (g) a dialkylamino group; or (h) a hydroxyl group. More specifically, the R¹⁰ substituent group may be a hydroxyl group, a 1 to 17 carbon alkyl group, a 3 to 6 carbon cycloalkyl group, a 1 to 2 ring aralkyl group, a 1 to 2 ring aryl group, a 1 to 10 carbon alkoxy group, a 1 to 6 carbon alkylamino group or a 1 to 6 carbon dialkylamino group. For example, the R¹⁰ substituent group may be either a methyl group (as illustrated in Examples 1-11 and 14-24) or tert-butoxy group comprising of the chemical formula OC(CH₃)₃ wherein the addition of the tert-butoxy group forms a BOC group at the N terminus of the molecule (as illustrated in Example 12).

X⁴ can be either an oxygen atom, a sulfur atom, or two hydrogen atoms.

R² is substituent selected from one of the following: (a) a hydrogen atom; (b) an alkyl group; (c) a hydroxyl group; (d) an alkoxy group, (e) a halo group (f) an amino group; (g) an alkylamino group; (h) a dialkylamino group (i) a carboxylic acid ester, (j) an amide group, or (k) an aralkyloxy group. More specifically, the substituent group of R² is a hydrogen atom, a 1 to 6 carbon alkyl group, a hydroxyl group, a 1 to 10 carbon alkoxy group, a halo group, an amine group, a 1 to 6 carbon alkylamino group, a 1 to 6 carbon dialkylamino group, a carboxylic acid ester, an amide group, a 1 to 2 ring aralkyloxy group, or a 1 to 2 ring aralkyloxy group. In one embodiment, for example, the substituent group of R² is a hydrogen atom (as illustrated in Example 13). In another embodiment, the substituent group of R² forms a cyclic unit, e.g. a phenyl ring, in conjunction with R³ (as illustrated in Examples 8-9).

R³ is a substituent selected from one of the following: (a) a hydrogen atom; (b) an alkyl group; (c) an alkoxy group; (d) a hydroxyalkyl group; (e) an aralkyloxy group; (f) an aryloxy group; (g) a halo group; (h) a sulfidryl group; (i) an alkylsulfidryl group; (j) a primary amine group; (k) a secondary amine group; (l) a tertiary amine group; (m) an alkylamino group; (n) a dialkylamino group; (o) a carboxylic acid ester; (p) an amide group, or (q) a hydroxyl group. More specifically, R³ is a substituent group comprised of a hydrogen atom, a hydroxyl group, a 1 to 6 carbon alkyl group, a 1 to 10 carbon alkoxy group, a 1 to 6 carbon hydroxyalkyl group, a 1 to 2 ring aralkyloxy group, a 1 to 2 ring aryloxy group, a halo group, a sulfidryl group, an alkylsulfidryl group, an amine group, a 1 to 6 carbon alkylamino group, a 1 to 6 carbon dialkylamino group, a carboxylic acid ester, an amide group, or a substituent group comprising Formula II(b):

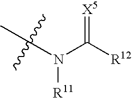

wherein R¹¹ is a hydrogen atom or a 1 to 6 carbon alkyl group; R¹² may be hydroxyl group, a 1 to 17 carbon alkyl group, a 1 to 10 carbon alkoxy group, a 1 to 2 ring aralkyloxy group, a 1 to 2 ring aryloxy group, a 1 to 6 carbon alkylamino group, or a 1 to 2 ring aralkylamino; and X⁵ is selected from the group consisting of an oxygen atom, a sulfur atom, and two hydrogen atoms.

In one embodiment, the substituent group of R² is capable of forming a cyclic unit, such as but not limited to a phenyl ring, in conjunction with R³ (as illustrated in Examples 8-9) or R⁴.

Based on the above, the substituent groups of R³ may be divided into two groups (1) groups bound at the R³ position by either a carbon or a hydrogen and (2) groups bound at the R³ position by a heteroatom. To this end, in one embodiment R³ may be comprised of a hydrogen group, a hydroxyalkyl group, an alkyl group a carboxylic acid ester, or an amide group, which may be represented by Formula III(B):

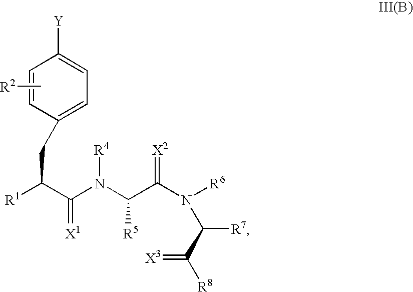

wherein Y represents a hydrogen group, a hydroxyalkyl group a carboxylic acid ester, an amide group, or an alkyl group wherein the hydrogen atom or a carbon atom in any of the above groups is bound at the Y position. For example, Y may be a hydrogen atom (as illustrated in Example 13), an alkyl group forming a phenyl ring structure with R² (as illustrated in Examples 8-9), or any similar embodiment in accordance with the above groups.

In another embodiment, R³ may be a substituent group bound at the R³ position by a heteroatom wherein a heteroatom is an atom other than carbon or hydrogen such as, but not limited to, a nitrogen atom, a sulfur atom, an oxygen atom, a fluorine atom, a bromine atom, a chlorine atom, and a iodine atom. To this end, this embodiment comprises an alkoxy group, a aralkyloxy group; an aryloxy group; a halo group; a sulfidryl group; an alkylsulfidryl group; a primary amine; a secondary amine; a tertiary amine; an alkylamino group; a dialkylamino group; or a hydroxyl group and is represented by Formula III(C):

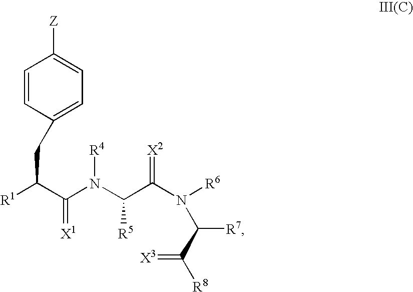

wherein Z represents an alkoxy group, a aralkyloxy group; an aryloxy group; a halo group; a sulfidryl group; an alkylsulfidryl group; a primary amine; a secondary amine; a tertiary amine; an alkylamino group; a dialkylamino group; or a hydroxyl group bound at the R3 position by a heteroatom. For example, Z may be a fluorine atom (as illustrated in Structures 10-11), a benzoyl group (as illustrated in Examples 2, 3, 5, 14, 17, 20, and 21), a hydroxyl group (as illustrated in Examples 1, 4, 12, 15, 16, 18, 19, 23 and 24) or a substituent group having the

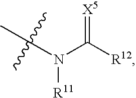

wherein R¹¹ is a hydrogen and R¹² is a benzoyl group (as illustrated in Examples 6 and 7).

R⁴ is either (a) a hydrogen atom or (b) an alkyl group. More specifically, R⁴ may be a hydrogen atom or a 1 to 6 carbon alkyl group wherein the alkyl group may be a chain molecule or exist as a cyclic ring structure in conjunction with a substituent group of G, R³ or R². For example, in one embodiment, R⁴ is an alkyl group forming a cyclic ring structure with R³ wherein R³ is comprised of Formula IV and the substituent group of R⁴ is attached at the R¹¹ position or to a substituent group at the R¹¹ position. However, this embodiment is not intended to be limiting to the present invention.

R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group.

For example, in one non-limiting embodiment, R⁵ is an isobutyl group forming a Leucine amino acid (See Examples 1-21). In another embodiment, R⁵ is a sec-butyl group forming an Isoleucine amino acid (See Example 23 and 24). In another embodiment, R⁵ is an amine group having the formula (CH₂)₄NH₂, or variant thereof, forming the side chain of Isoleucine amino acid. As used herein, a variant of the Isoleucine side chain includes the replacement of one or more hydrogens of the amine group with one or more organic substituent groups. For example, in one non-limiting embodiment, a carboxybenzyl group may replace a hydrogen of the amine group thereby forming a group of the formula (CH₂)₄NHC(O)OCH₂Ph (See Example 22). In another embodiment, R⁵ can be carbamate. In yet another embodiment, R⁵ is a lysine, an Ornithine or an aminoalkyl group.

R⁶ is a substituent group selected from one of the following: (a) a hydrogen atom or (b) an alkyl group. More specifically, R⁶ is a substituent group selected from the group consisting of a hydrogen atom and a 1 to 6 carbon alkyl group. For example, in one non-limiting embodiment, the R⁶ substituent group is capable of forming a cyclic ring structure in conjunction with an R⁷ substituent group. In a further embodiment, the cyclic ring structure formed between R⁶ and R⁷ may be a cyclic structure having from four to eight atoms wherein one of the atoms is a nitrogen. The cyclic structure may comprise the side chain of the amino acid Proline (as illustrated in examples 1-22). Alternatively, the R⁶ may be a hydrogen (as illustrated in examples 23 and 24).

R⁷ is a substituent selected from one of the following: (a) a hydrogen atom; (b) an alkyl group; (c) an aralkyl group; (d) a hydroxyalkyl group (e) a cycloalkyl group, (f) used in conjunction with R⁷ to form a cyclic unit. More specifically, R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group. To this end, in one embodiment, the R⁷ substituent group is capable of forming a cyclic ring structure in conjunction with an R⁶ substituent group. In a further embodiment, the cyclic ring structure formed between R⁷ and R⁶ may be a cyclic structure having from four to eight atoms wherein one of the atoms is a nitrogen. For example, the cyclic structure is the side chain of the amino acid Proline (as illustrated in the examples 1-22). Alternatively, the R⁷ group is a hydroxyalkyl group such as a hydroxymethyl group (OH—CH₂—) thereby forming a Serine amino acid (as illustrated in examples 23 and 24).

R⁸ is a substituent group selected from a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a second-ary amine, a tertiary amine, a tertiary cyclic amine optionally substituted by a guanidinylalkyl group, a 1 to n carbon aminoalkyl-amino group where n=2-6 and derivatives thereof such as an amide, a carbamate, a urea, an amidine and a guanidine, an aminoalkyl group and derivatives thereof such as an amide, a carbamate, a urea, an amidine and a guanidine. Amino-alkylamino R⁸ groups are represented generally by the formula NH(CH₂)ₙNH₂, with the aforementioned derivatives represented by formulas such as NH(CH₂)ₙNHC(O)Me, NH(CH₂)ₙNHC(═NH)NH₂, and the like. In at least one embodi-ment, the R⁸ substituent comprises a peptide moiety having 1-4 amino acids bound at the R⁸ position by an N-terminus end of the peptide moiety. Additional substituent groups may extend from an N terminus, a side chain and/or a C terminus of each amino acid within the peptide moiety wherein the additional substituent groups may be selected from the group consisting of a hydrogen atom, a hydroxyl group, a heterocyclic aromatic compound, an alkyl ester group, a cycloalkyl group, an amino alkyl group, an aminoalkylamino group, a primary amine, a secondary amine, and a tertiary amine. The peptide moiety may be a 1-4 natural or unnatural amino acids with at least one additional substituent group extending from the peptide.

In one embodiment, R⁸ is a substituent group which is not a peptide moiety. To this end, the R⁸ substituent group is selected from the group consisting of a hydroxyl group, a 1 to 10 carbon alkoxy group, a 1 to 2 ring aralkyloxy group, a 1 to 6 carbon alkylamino group, a 1 to 6 carbon dialkylamino group, a carbamate, or a guanidine, a tertiary cyclic amine optionally substituted by a guanidinylalkyl group, a primary amine, a secondary amine, and a tertiary amine, each of which is bound at the R⁸ position by a heteroatom. For example, R⁸ may be a methoxy group (as illustrated in Examples 2, 6, 8, 10, and 23), a hydroxyl group (as illustrated in Examples 3, 7, 9, 11, 22 and 24), or a 1,4 diaminobutane group (as illustrated in Example 5) each of which may be bound at the R⁸ position by a heteroatom. In another embodiment, the present invention provides a compound of Formula III(C), wherein Z represents a hydroxyl or aralkyloxy group, for example, benzyloxy; X¹, X², X³ and X⁴ are each oxygen (O); R¹ a substituent group comprising Formula II(a):

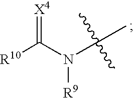

R⁴ is hydrogen; R⁵ is a C₁₋₄ alkyl; R⁶ and R⁷ together, along with the nitrogen and carbon atoms to which they are attached, respectively, form a five-membered ring; R⁸ is 1-piperidinyl or pyrrolidinyl optionally substituted by a guanidinylalkyl group; R⁹ is hydrogen; and R¹⁰ is C₁₋₈ alkyl.

In another embodiment, the small molecules of the present invention are defined by the Formula III(D):

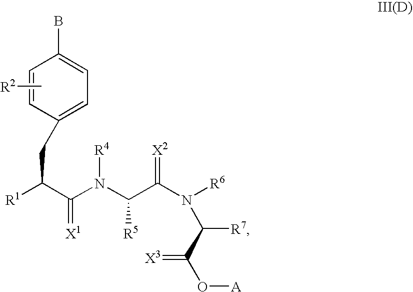

wherein O is an oxygen bound at the R⁸ position and, when A is a hydrogen atom, B can generally be selected from the group consisting of a hydrogen atom; a hydroxyl group; an alkyl group; an alkoxy group; a hydroxyalkyl group; an aralkyloxy group; an aryloxy group; a halo group; a sulfidryl group; an alkylsulfidryl group; an amino acid; an alkylamino group; a hydroxyl group; or a dialkylamino group.

However, in a most preferred embodiment, if A is a hydrogen, then B must be selected from the group consisting of a hydrogen, an alkyl group, an alkoxy group, a hydroxyalkyl group, a halo group, an aralkyloxy group, an aryloxy group, or an alkylamino group. To this end, if R⁸ is a hydroxyl group then the R³ group is preferably not a hydroxyl group.

In another embodiment, the R⁸ substituent may be a peptide moiety bound at the R⁸ position by a heteroatom such as the nitrogen atom of the N terminus of the peptide moiety. For example, R⁸ may be at least one amino acid wherein the amino acid is an Ornithine or a variant of Ornithine bound to the R⁸ position by its N-terminus. As contemplated herein, a variant of Ornithine may be the addition or subtraction of additional substituent groups from the N terminus, C terminus and/or side chain of the amino acid. A variant may also include a modification, addition, or subtraction of carbon atoms to the side chain of the Ornithine amino acid.

The additional substituent groups which may be added to the Ornithine may be one or more hydrogen atoms, alkyl groups, hydroxyl groups, heterocyclic aromatic compounds, alkyl ester groups, cycloalkyl groups, amino alkyl groups, aminoalkylamino groups, primary amines, secondary amines, and tertiary amines. To this end, this embodiment is represented by Formula III(E):

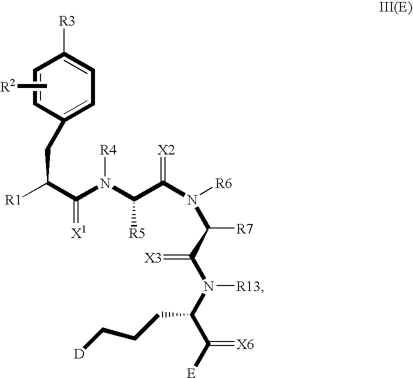

wherein additional group R¹³ is a hydrogen atom, a hydroxyl group, a heterocyclic aromatic compound, an alkyl ester group, a cycloalkyl group, an amino alkyl group, an aminoalkylamino group, a primary amine, a secondary amine, or a tertiary amine. Additional group D is selected from the group consisting of a hydrogen atom, a hydroxyl group, a heterocyclic aromatic or non-aromatic compound, an alkyl ester group, a cycloalkyl group, an aminoalkyl group, an aminoalkylamino group, an alkylamino, an amide including a carbamate and an urea, a guanidine, a guanidinyl, a guanidinium, a primary amine, a secondary amine, and a tertiary amine. Additional group, X⁶ is selected from the group consisting of an oxygen atom, a sulfur atom, and two hydrogen atoms. Additional group E is selected from the group consisting of a hydrogen atom, a hydroxyl group, a heterocyclic aromatic compound, an alkyl ester group, a cycloalkyl group, an amino alkyl group, an aminoalkylamino group, a primary amine, a secondary amine, or a tertiary amine.

To this end, in one non-limiting embodiment, R¹³ is a hydrogen and D is a carbamate, a guanidine, guanidinium, a guanidinyl or substituted guanidinyl group, so as to form the side chain structure of Arginine (as illustrated in Examples 1, 4, and 12-16). Additional substituent D, however, are not limited to this embodiment and may comprise such functional groups so as to form an Ornithine amino acid (as illustrated in Examples 19 and 20). Alternatively, substituent D may comprise a BOC group or a 2-pyrimidine group so as to form an Ornithine variant (as illustrated in Examples 17, 18, and 21).

In other embodiments of the present invention, additional substituent group E may be a primary amine, such as NH₂ (as illustrated in Example 1), a secondary alkylamine, such as NHC₄H₉ (as illustrated in Example 15), a secondary amine with a cycloalkyl structure, such as NH(CH₂)₃Ph (as illustrated in Examples 12 and 16-21), or a cyclo tertiary amine such as N(CH₂)₅ (as illustrated in Examples 4 and 14).

In another embodiment, the small molecules of the present invention are represented by Formula III(E), wherein R¹ a substituent group comprising Formula II(a):

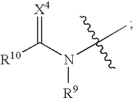

R² is hydrogen; R³ is —OH or —Obzl; R⁴ is hydrogen; R⁵ is C₁₋₄ alkyl; R⁶ and R⁷ together, along with the nitrogen and carbon atoms to which they are attached respectively, form a five-membered ring; R⁹ is hydrogen; and R¹⁰ is C₁₋₈ alkyl; X¹, X², X³ and X⁴ are each oxygen (O); D is guanidinyl; and E is amino (NH₂), C₁₋₄ alkoxy, or a tertiary cyclic amine.

In another embodiment, the small molecules of the present invention are represented by Formula III(F):

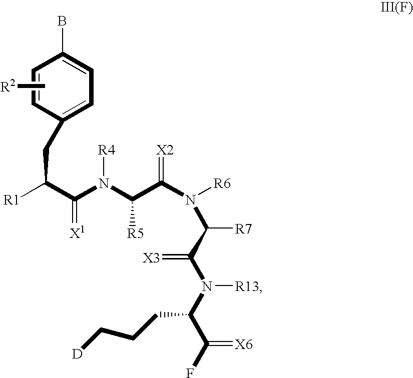

wherein F may be a hydroxyl group, a primary amine, or an alkoxy group and B may generally be selected from the group consisting of a hydrogen atom; a hydroxyl group; an alkyl group; an alkoxy group; a hydroxyalkyl group; an aralkyloxy group; an aryloxy group; a halo group; a sulfidryl group; an alkylsulfidryl group; an amino acid; an alkylamino group; an aminoalkyl or a dialkylamino group. In a more preferred embodiment, however, if F is a hydroxyl group, a primary amine, or an alkoxy group then B must be selected from the group consisting of a hydrogen, an alkyl group, an alkoxy group, a hydroxyalkyl group, a halo group, an aralkyloxy group, an aryloxy group, or an alkylamino group. To this end, if the additional substituent attached to the C terminus of the peptide moiety (represented by F in Formula III(F)) is a primary amine (NH₂), a hydroxyl group, or an alkoxy group, then the R³ group is preferably not a hydroxyl group.

In a further embodiment of the present invention, the variant of Ornithine may be formed from the addition or subtraction of carbon atoms from the Ornithine side chain. For example, the Ornithine side chain of Formula III(E) may comprise one additional carbon, thereby, forming a Lysine amino acid, or variant thereof, as illustrated in Formula III(E)(i):

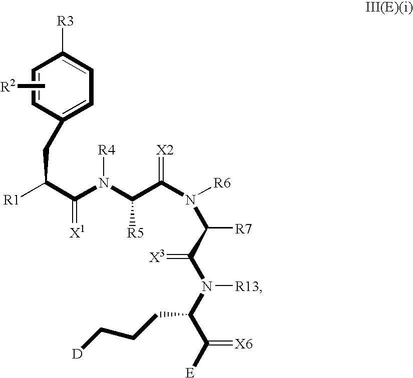

Alternatively, one carbon may be removed from the side chain of the Ornithine variant of Formula III(E), thereby forming Formula III(E)(ii):

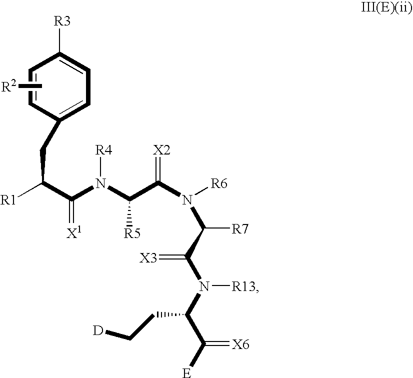

In an even further alternative, two carbons may be removed from the side chain of the Ornithine variant of Formula III(E), thereby, forming Formula III(E)(iii)

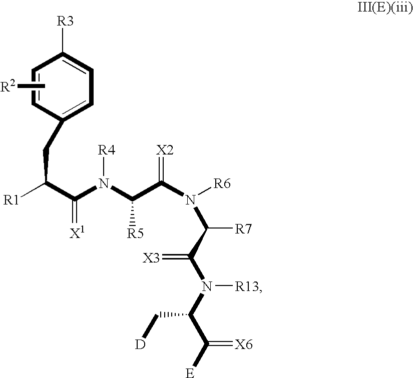

In another embodiment, the R⁸ group is a two amino acid peptide moiety comprising Ornithine, or a variant thereof, and/or Proline wherein the N terminus of the Ornithine or Ornithine variant is bound at the R⁸ position and the N terminus of the Proline is bound to the C terminus of the Ornithine or Ornithine variant. The Proline has at least one additional substituent group extending from at least its C terminus. The additional substituent group may be a hydrogen atom, a hydroxyl group, a heterocyclic aromatic or non-aromatic compound, an alkyl ester group, a cycloalkyl group, an aminoalkyl group, a carbamate, guanidinyl, a guanidinium or a substituted guanidinyl group, a primary amine, a secondary amine, and a tertiary amine. For example, this embodiment is represented by Formula III(G):

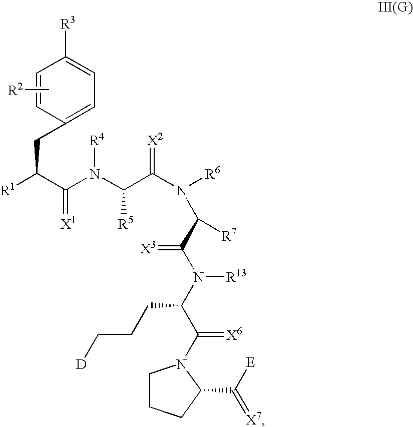

wherein additional substituents R¹³, and D is selected from the group consisting of a hydrogen atom, a hydroxyl group, a heterocyclic aromatic compound, an alkyl ester group, a cycloalkyl group, an aminoalkyl group, an aminoalkylamino group, alkylamino, an amide, a carbamate, an urea, a guanidine, a primary amine, a secondary amine, and a tertiary amine. X⁷ is selected from the group consisting of an oxygen atom, a sulfur atom, and two hydrogen atoms. E is selected from the group consisting of a hydrogen atom, a hydroxyl group, a heterocyclic aromatic compound, an alkyl ester group, an alkylamino, a cycloalkyl group, an aminoalkyl group, an aminoalkylamino group, a primary amine, a secondary amine, and a tertiary amine.

To this end, the E substituent group may be a primary amine, such as NH₂ (as illustrated in Example 1), a secondary alkylamine, such as NHC₄H₉ (as illustrated in Example 15), a secondary amine with a cycloalkyl structure, such as NH(CH₂)₃Ph (as illustrated in Examples 12 and 16-21), or a cyclo tertiary amine such as N(CH₂)₅ (as illustrated in Examples 4 and 14).

In another embodiment, the small molecules of the present invention are represented by Formula III(H):

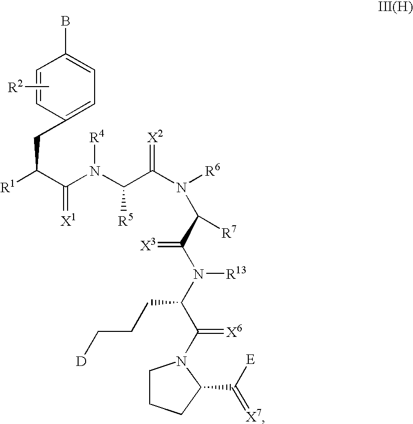

wherein F is a hydroxyl group, a primary amine, or an alkoxy group and B may then be a hydrogen atom; a hydroxyl group; an alkyl group; an alkoxy group; a hydroxyalkyl group; an aralkyloxy group; an aryloxy group; a halo group; a sulfidryl group; an alkylsulfidryl group; an amino acid; an alkylamino group; or a dialkylamino group. In the most preferred embodiment of this aspect of the invention, however, if F is a hydroxyl group, a primary amine, or an alkoxy group then B must be selected from the group consisting of a hydrogen, an alkyl group, an alkoxy group, a hydroxyalkyl group, a halo group, an aralkyloxy group, an aryloxy group, or an alkylamino group. To this end, if the additional substituent group at the F position is a primary amine, a hydroxyl group, or an alkoxy group, then the R³ group is preferably not a hydroxyl group.

In one embodiment, the two peptide moiety is comprised of an Arginine-Proline moiety. To this end, D is a guanidinyl, a guanidinium, or a substituted guanidinyl group, and in the case of R⁸ being Arginine, D has the formula:

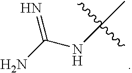

The two peptide moiety, however, is not limited to an Arginine-Proline moiety. In a further embodiment, R⁸ is a substituent group comprising an unnatural amino acid in conjunction with at least one natural amino acid. For example, the unnatural amino acid is a variant of the amino acid structure of Ornithine. To this end, referring to Formula III(G), the unnatural amino acid may be Ornithine wherein additional substituent groups D is a carbamate comprising the general formula:

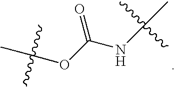

Furthermore, the natural amino acid may be Proline wherein the N-terminus end of the Ornithine is bound at the R⁸ position. The present invention is not limited to a standard Ornithine amino acid structure, as disclosed above, and may include variants and/or derivatives of the Ornithine. For example, referring again to Formula III(G), additional substituent D may comprise a BOC substituent group (as illustrated in Examples 17 and 18) or a 2-pyrimidine substituent group (as illustrated in Example 21). Yet, the additional substituent group D is not limited to these embodiments and may be any of the embodiments disclosed herein.

In a further embodiment of the present invention, the variant of Ornithine in the two peptide moiety may be formed from the addition or subtraction of carbon atoms from the side chain. For example, the Ornithine side chain may be one additional carbon, thereby, forming a Lysine amino acid, as illustrated in Formula III(G)(i)

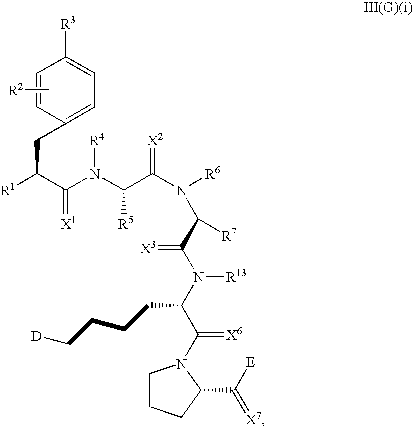

Alternatively, one carbon may be removed from the side chain of Ornithine, thereby forming Formula III(G)(ii):

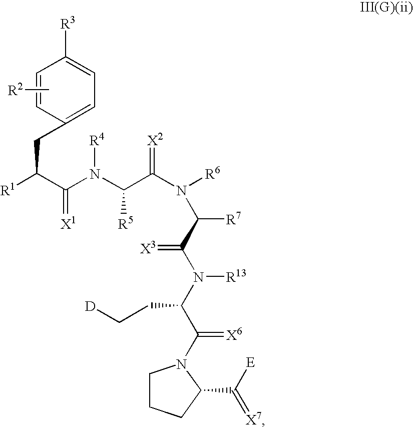

In an even further alternative, two carbons are removed from the side chain of Ornithine, thereby, forming Formula III(G)(iii)

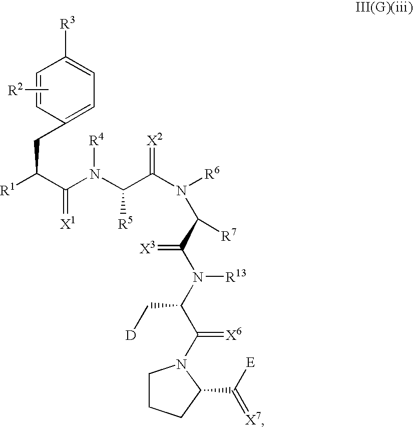

Each of the above embodiments illustrate the various possible compounds of which the substituent groups may comprise in Formulas I-IV. To this end, each of these compounds may be in any combination so as to form a compound of the present invention. The following exemplary compounds are not intended to limit the possible compounds associated with the present invention. Rather, they are set forth so as to illustrate several possible compounds contemplated by the present invention and consistent with the above.

In one non-limiting embodiment, the present invention has the structure of Formula I wherein G comprises a substituted benzyl group hereby forming Formula III. The X¹, X², and X³ groups of the small molecule are oxygen atoms. The R¹ group is defined by the structure:

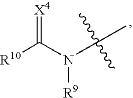

wherein X⁴ is an oxygen atom, R⁹ is a hydrogen atom, and R¹⁰ is a methyl group. The R² and R⁴ groups are hydrogen atoms. The R³ group is a hydroxyl group. The R⁵ group is an isobutyl group. The R⁶ and R⁷ groups are comprised of alkyl groups which form a cyclic ring structure therebetween. Finally, the R⁸ group is comprised of an Arg-Pro peptide wherein the N terminus of the Arginine is bound to the structure at the R⁸ position. The resulting C terminus of the Proline is further bound to a primary amine, NH₂. To this end, the resulting chemical structure (as illustrated in Example 1) is the following:

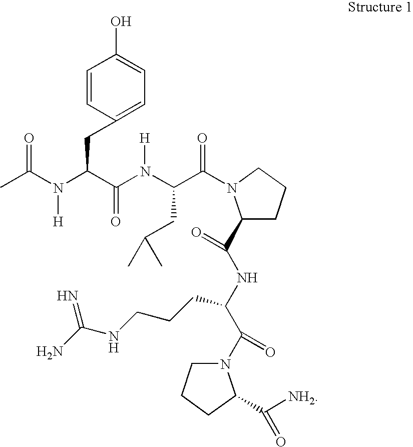

In another non-limiting embodiment, the present invention comprises of Formula I wherein G is a substituted benzyl group thereby forming Formula III. The X¹, X², and X³ groups are oxygen atoms. The R¹ group is defined by Formula II(a):

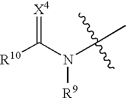

wherein X⁴ is an oxygen atom, R⁹ is a hydrogen atom, and R¹⁰ is a methyl group. The R² and R⁴ groups are preferably hydrogen atoms. The R³ group is a benzoyl group. The R⁵ group is an isobutyl group. The R⁶ and R⁷ groups are comprised of alkyl groups which form a cyclic ring structure therebetween. Finally, R⁸ is a methoxy group (OCH3). To this end, the resulting chemical structure (as illustrated in Example 2) is the following:

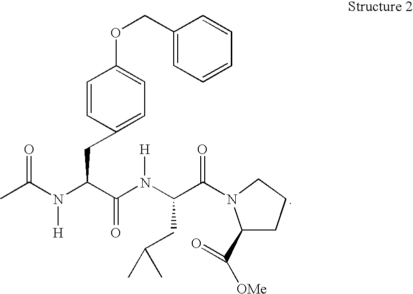

In another non-limiting embodiment, the present invention is represented by the structure of Formula I wherein G is a substituted benzyl group thereby forming Formula III. The X¹, X², and X³ groups are oxygen atoms. The R¹ group is represented by the structure of Formula II(a):

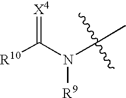

wherein X⁴ is an oxygen atom, R⁹ is a hydrogen atom, and R¹⁰ is a methyl group. The R² and R⁴ groups are hydrogen atoms. The R³ group is a benzoyl group. The R⁵ group is an isobutyl group. The R⁶ and R⁷ groups are alkyl groups which form a cyclic ring structure therebetween. Finally, R⁸ is a hydroxyl group (OH). To this end, the resulting chemical structure (as illustrated in Example 3) is the following:

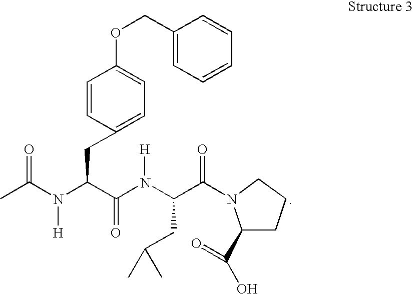

In another non-limiting embodiment, the present invention is comprised of Formula I wherein G is a substituted benzyl group thereby forming Formula III. The X¹, X², and X³ groups are oxygen atoms. The R¹ group is represented by Formula II(a):

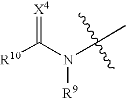

wherein X⁴ is an oxygen atom, R⁹ is a hydrogen atom, and R¹⁰ is a methyl group. The R² and R⁴ groups are comprised of hydrogen atoms. The R³ group is a hydroxyl group. The R⁵ group is an isobutyl group. The R⁶ and R⁷ groups are each an alkyl group which form a cyclic ring structure therebetween. Finally, the R⁸ group is an Arginine amino acid wherein the N terminus end of the amino acid is bound at the R⁸ position. The C terminus of the Arginine is further bound to a cyclic tertiary amine comprising the formula N(CH₂)₅ wherein, in one embodiment, the nitrogen atom of the cyclic amine is bound to the C terminus of the Arginine. To this end, the resulting chemical structure (as illustrated in Example 4) is the following:

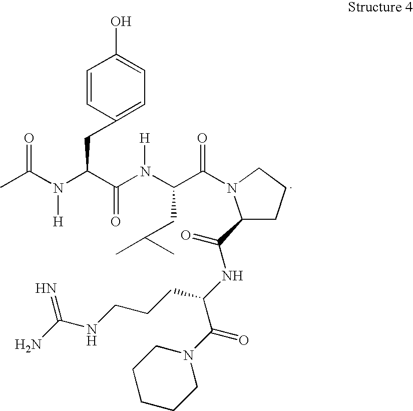

In another non-limiting embodiment, the present invention is represented by structure of Formula I, wherein G comprises a substituted benzyl group thereby forming Formula III. The X¹, X², and X³ groups are oxygen atoms. The R¹ the alkyl group is represented by the structure of Formula II(a):

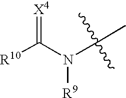

wherein X⁴ is an oxygen atom, R⁹ is a hydrogen atom, and R¹⁰ is methyl group. The R² and R⁴ groups are hydrogen atoms. The R³ group is a benzoyl group. The R⁵ group is an isobutyl group. The R⁶ and R⁷ groups are each an alkyl group which form a cyclic ring structure therebetween. Finally, R⁸ is a 1,4 diaminobutyl group (NHC₄H₈NH) wherein at least one of the nitrogen atoms of the 1,4 diaminobutyl group is bound at the R⁸ position. To this end, the resulting chemical structure (as illustrated in Example 5) is the following:

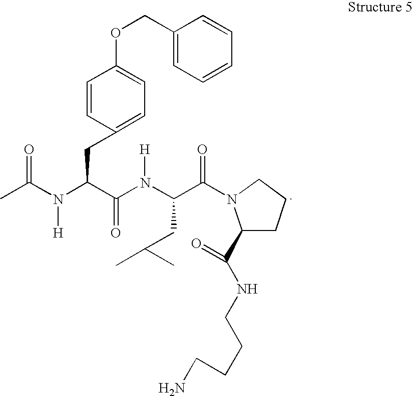

In another non-limiting embodiment, the present invention is represented by the structure of Formula I, wherein G is a substituted benzyl group thereby forming Formula III. The X¹, X², and X³ groups are each an oxygen atom. The R¹ group is represented by the Formula II(a):

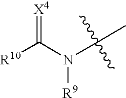

wherein X⁴ is an oxygen atom, R⁹ is a hydrogen atom, and R¹⁰ is a methyl group. The R² and R⁴ groups are independently hydrogen atoms. The R³ group is represented by Formula II(b):

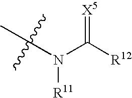

wherein X⁵ is an oxygen, R¹¹ is a hydrogen atom and R¹² is a benzoyl group. The R⁵ group comprises an isobutyl group. The R⁶ and R⁷ groups are each an alkyl group which form a cyclic ring structure therebetween. Finally, R⁸ is a methoxy group (OCH3). To this end, the resulting chemical structure (as illustrated in Example 6) is the following:

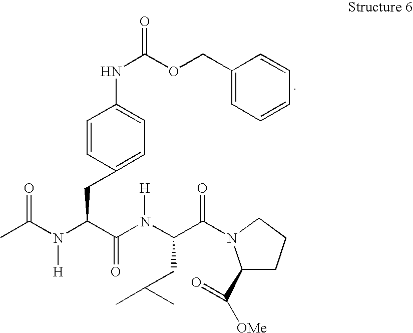

In another non-limiting embodiment, the present invention is represented by Formula I wherein G comprises a substituted benzyl group thereby forming Formula III. The X¹, X², and X³ groups are oxygen atoms. The R¹ group is comprised of Formula II(a):

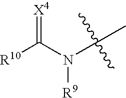

wherein X⁴ is an oxygen atom, R⁹ is a hydrogen atom, and R¹⁰ is a methyl group. The R² and R⁴ groups are each a hydrogen atom. The R³ group is represented by the structure of Formula II(b):

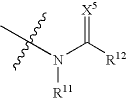

wherein X⁵ is an oxygen atom, R¹¹ is a hydrogen atom and R¹² is a benzoyl group. The R⁵ group is an isobutyl group. The R⁶ and R⁷ groups are each an alkyl group which form a cyclic ring structure therebetween. Finally, R⁸ is a hydroxyl group. To this end, the resulting chemical structure (as illustrated in Example 7) is the following:

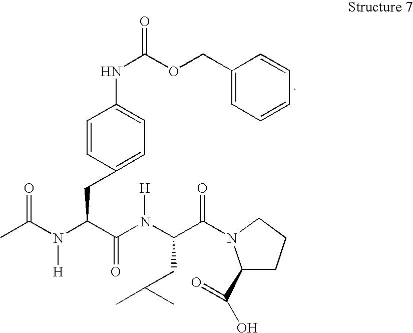

In another non-limiting embodiment, the present invention is represented by the structure of Formula I, wherein G is a substituted benzyl group thereby forming Formula III. The X¹, X², and X³ groups are oxygen atoms. The R¹ group is represented by the structure of Formula II(a):

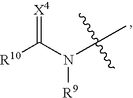

wherein X⁴ is an oxygen atom, R⁹ is a hydrogen atom, and R¹⁰ is a methyl group. The R² and R³ groups form an aryl group therebetween. The R⁴ group is a hydrogen atom. The R⁵ group is an isobutyl group. The R⁶ and R⁷ groups are each an alkyl group which form a cyclic ring structure therebetween. Finally, R⁸ is comprised of a methoxy group (OCH3). To this end, the resulting chemical structure (as illustrated in Example 8) is the following:

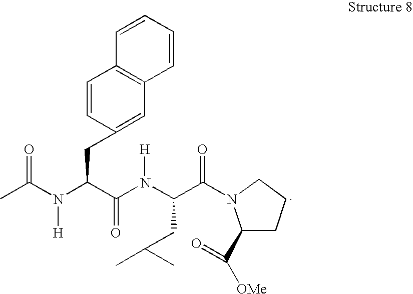

In another non-limiting embodiment, the present invention is represented by the structure of Formula I, wherein G is a substituted benzyl group thereby forming Formula III. The X¹, X², and X³ groups are oxygen atoms. The R¹ group is represented by the structure of Formula II(a):

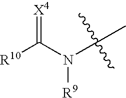

wherein X⁴ is an oxygen atom, R⁹ is a hydrogen atom, and R¹⁰ is a methyl group. The R⁴ group is a hydrogen atom. The R² and R³ groups form an aryl group therebetween. The R⁵ group comprises an isobutyl group. The R⁶ and R⁷ groups are each an alkyl group which form a cyclic ring structure therebetween. Finally, R⁸ is a hydroxyl group. To this end, the resulting chemical structure (as illustrated in Example 9) is the following:

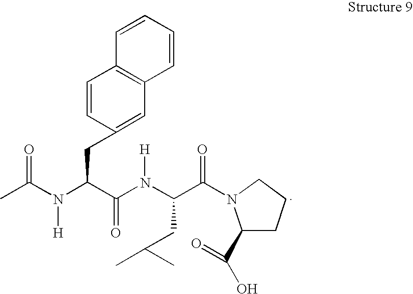

In another non-limiting embodiment, the present invention is represented by Formula I, wherein G is comprised of a substituted benzyl group thereby forming Formula III. The X¹, X², and X³ groups are oxygen atoms. The R¹ group is represented by the structure of Formula II(a):

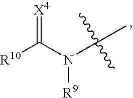

wherein X⁴ is an oxygen atom, R⁹ is a hydrogen atom, and R¹⁰ is a methyl group. The R² and R⁴ groups are hydrogen atoms. The R³ group is a fluorine group. The R⁵ group is an isobutyl group. The R⁶ and R⁷ groups are each alkyl groups which form a cyclic ring structure therebetween. Finally, R⁸ is a methoxy group (OCH3). To this end, the resulting chemical structure (as illustrated in Example 10) is the following:

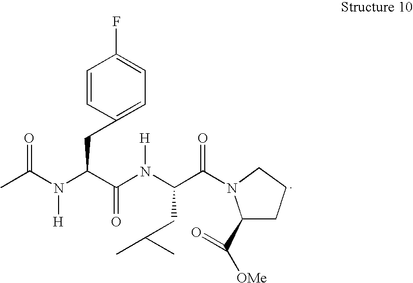

In another non-limiting embodiment, the present invention is represented by the structure of Formula I wherein G is a substituted benzyl group thereby forming Formula III. The X¹, X², and X³ groups are oxygen atoms. The R¹ group is represented by the structure of Formula II(a):

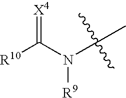

wherein X⁴ is an oxygen atom, R⁹ is a hydrogen atom, and R¹⁰ is a methyl group. The R² and R⁴ groups are each hydrogen atoms. The R³ group is a fluorine group. The R⁵ group is an isobutyl group. The R⁶ and R⁷ groups are each an alkyl group which form a cyclic ring structure therebetween. Finally, R⁸ is a hydroxyl group. To this end, the resulting chemical structure (as illustrated in Example 11) is the following:

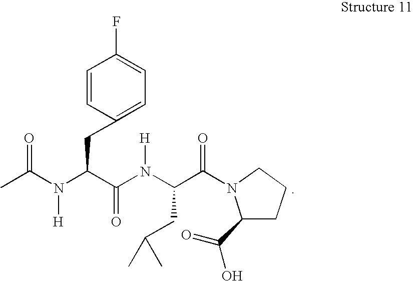

In another non-limiting embodiment, the present invention is represented by the structure of Formula I, wherein G is a substituted benzyl group thereby forming Formula III. The X¹, X², and X³ groups are oxygen atoms. The R¹ the alkyl group is represented by the structure of Formula II(a):

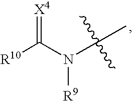

wherein X⁴ is an oxygen atom, R⁹ is a hydrogen atom, and R¹⁰ is a tert-butoxy group (OCC₃H₉) wherein the tert-butoxy group is added at the R¹⁰ position to form a BOC group at the N-terminus of the small molecule. The R² and R⁴ groups are hydrogen atoms. The R³ group is a hydroxyl group. The R⁵ group is an isobutyl group. The R⁶ and R⁷ groups are each an alkyl group which form a cyclic ring structure therebetween. Finally, the R⁸ group is an Arg-Pro peptide wherein the N terminus end of the Arginine is bound at the R⁸ position. The resulting C terminus of the Proline is further bound to a secondary amine with a cycloalkyl substituent such as NH(CH₂)₃Ph wherein the nitrogen atom of the amine is bound to the C-terminus of the Proline. To this end, the resulting chemical structure (as illustrated in Example 12) is the following:

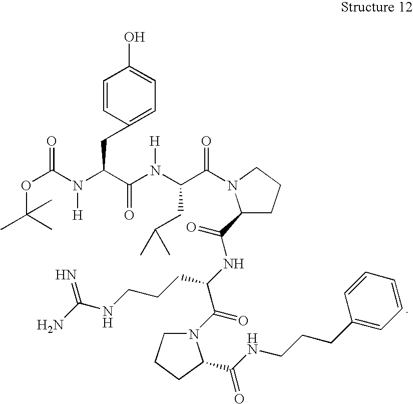

In another non-limiting embodiment, the present invention is represented by the structure Formula I, wherein G is a substituted benzyl group thereby forming Formula III. The X¹, X², and X³ groups are oxygen atoms. The R¹, R², R³, and R⁴ groups are all hydrogen atoms. The R⁵ group is an isobutyl group. The R⁶ and R⁷ groups are each an alkyl group which form a cyclic ring structure therebetween. Finally, the R⁸ group is a derivative of an Arginine amino acid wherein the N terminus end of the amino acid is bound to the structure at the R⁸ position and the C terminus is further bound to a cyclic tertiary amine, piperidinyl, comprising the formula N(CH₂)₅ wherein the nitrogen atom of said amine is bound to the C-terminus of the Arginine. To this end, the resulting chemical structure (as illustrated in Example 13) is the following:

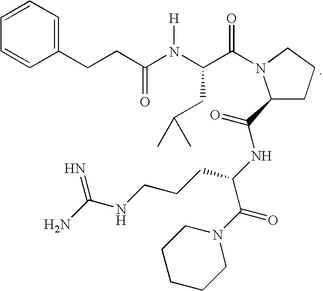

In another non-limiting embodiment, the present invention is represented by the structure of Formula I, wherein G is a substituted benzyl group thereby forming Formula III. The X¹, X², and X³ groups are oxygen atoms. The R¹ group is represented by the structure of Formula II(a):

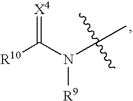

wherein X⁴ is an oxygen atom, R⁹ is a hydrogen atom, and R¹⁰ is a methyl group comprising the chemical formula CH₃. The R² and R⁴ groups are both hydrogen atoms. The R³ group is a benzoyl group. The R⁵ group is an isobutyl group. The R⁶ and R⁷ groups are each an alkyl group which form a cyclic ring structure therebetween. Finally, the R⁸ group is a derivative of Arginine amino acid wherein the N terminus end of the amino acid is bound to the structure at the R⁸ position, the C terminus is further bound to a cyclic tertiary amine, piperidinyl, comprising the formula N(CH₂)₅ wherein the nitrogen atom of said amine is bound to the C-terminus of the Arginine. To this end, the resulting chemical structure (as illustrated in Example 14) is the following:

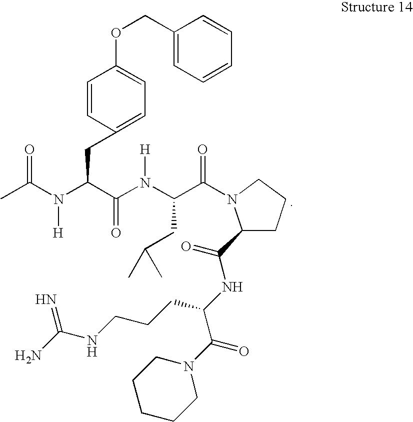

In another non-limiting embodiment, the present invention is represented by the structure of Formula I, wherein G is a substituted benzyl group thereby forming Formula III. The X¹, X², and X³ groups are oxygen atoms. The R¹ group is represented by the structure of Formula II(a):

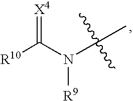

wherein X⁴ is an oxygen atom, R⁹ is a hydrogen atom, and R¹⁰ is a methyl group. The R² and R⁴ groups are hydrogen atoms. The R³ group is a hydroxyl group. The R⁵ group is an isobutyl group. The R⁶ and R⁷ groups are each an alkyl group which form a cyclic ring structure therebetween. Finally, the R⁸ group is an Arg-Pro peptide wherein the N terminus end of the Arginine is bound at the R⁸ position of the above structure. The resulting C terminus of the Proline is further bound to a secondary amine wherein the secondary amine is comprised of the formula: NHC₄H₉ wherein the nitrogen atom of the amine is bound to the C-terminus of the Proline. To this end, the resulting chemical structure (as illustrated in Example 15) is the following:

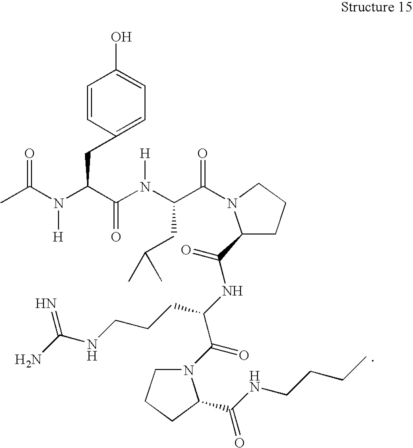

In another non-limiting embodiment, the present invention is represented by the structure of Formula I, wherein G is a substituted benzyl group thereby forming Formula III. The X¹, X², and X³ groups are oxygen atoms. The R¹ group is represented by the structure of Formula II(a):

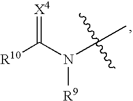

wherein X⁴ is an oxygen atom, R⁹ is a hydrogen atom, and R¹⁰ is a methyl group. The R² and R⁴ groups are each comprised of hydrogen atoms. The R³ group is a hydroxyl group. The R⁵ group is an isobutyl group. The R⁶ and R⁷ groups are each an alkyl group which form a cyclic ring structure therebetween. Finally, the R⁸ group is an Arg-Pro peptide wherein the N-terminus end of the Arginine is bound at the R⁸ position. The resulting C-terminus of the Proline is further bound to a secondary amine with a cycloalkyl substituent such as NH(CH₂)₃Ph wherein the nitrogen atom of the amine is bound to the C-terminus of the Proline. To this end, the resulting chemical structure (as illustrated in Example 16) is the following:

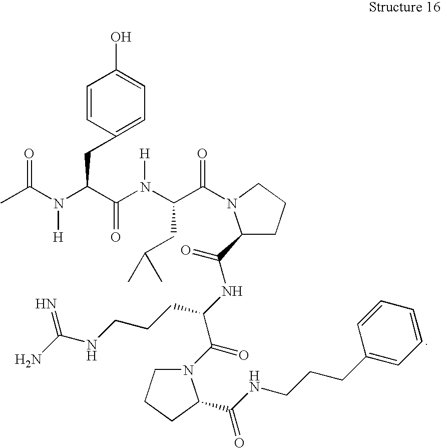

In another non-limiting embodiment, the present invention is represented by the structure of Formula I wherein G is a substituted benzyl group thereby forming Formula III. The X¹, X², and X³ groups are oxygen atoms. The R¹ group is the represented by Formula II(a):

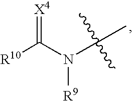

wherein X⁴ is an oxygen atom, R⁹ is a hydrogen atom, and R¹⁰ is a methyl group. The R² and R⁴ groups are hydrogen atoms. The R³ group is a benzoyl group. The R⁵ group is an isobutyl group. The R⁶ and R⁷ groups are each an alkyl group which form a cyclic ring structure therebetween. Finally, the R⁸ group is a (Boc)Orn-Pro peptide wherein the N terminus end of the Ornithine is bound to the structure at the R⁸ position of the above structure. The resulting C terminus of the Proline is further bound to a secondary amine with a cycloalkyl structure attached thereto such as NH(CH₂)₃Ph wherein the nitrogen atom of the amine is bound to the C-terminus of the Proline. To this end, the resulting chemical structure (as illustrated in Example 17) is the following:

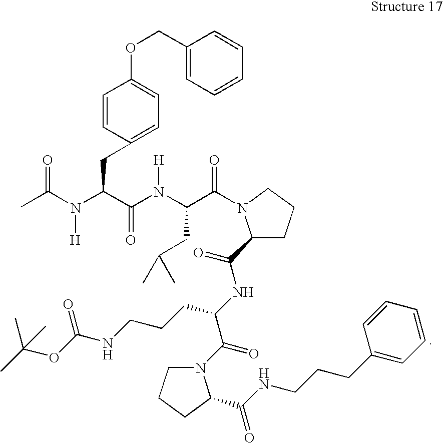

In another non-limiting embodiment, the present invention is represented by the structure of Formula I wherein G is a substituted benzyl group thereby forming Formula III. The X¹, X², and X³ groups are oxygen atoms. The R¹ group is represented by the structure of Formula II(a):

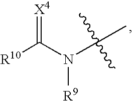

wherein X⁴ is an oxygen atom, R⁹ is a hydrogen atom, and R¹⁰ is a methyl group. The R² and R⁴ groups are hydrogen atoms. The R³ group is a hydroxyl group. The R⁵ group is a isobutyl group. The R⁶ and R⁷ groups are each an alkyl group which form a cyclic ring structure therebetween. Finally, the R⁸ group is a (Boc)Orn-Pro peptide wherein the N-terminus end of the Ornithine is bound to the structure at the R⁸ position of the above structure. The resulting C-terminus of the Proline is further bound to a secondary amine with a cycloalkyl structure attached thereto such as NH(CH₂)₃Ph wherein the nitrogen atom of the amine is bound to the C-terminus of the Proline. To this end, the resulting chemical structure (as illustrated in Example 18) is the following:

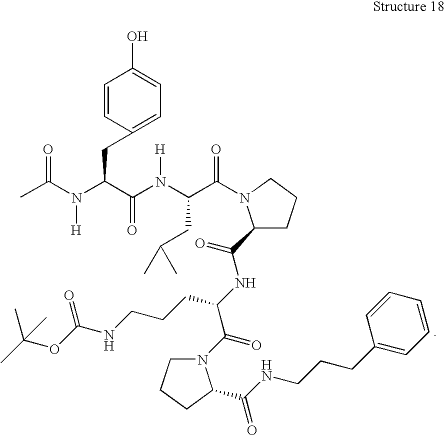

In another non-limiting embodiment, the present invention is represented by the structure of Formula I wherein G is a substituted benzyl group thereby forming Formula III. The X¹, X², and X³ groups are oxygen atoms. The R¹ group is represented by the structure of Formula II(a):

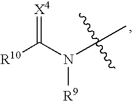

wherein X⁴ is an oxygen atom, R⁹ is a hydrogen atom, and R¹⁰ is a methyl group. The R² and R⁴ groups are hydrogen atoms. The R³ group is a hydroxyl group. The R⁵ group is an isobutyl group. The R⁶ and R⁷ groups are each an alkyl group which forms a cyclic ring structure therebetween. Finally, the R⁸ group is of an Orn-Pro peptide wherein the N terminus end of the Ornithine is bound to the structure at the R⁸ position of the above structure. The resulting C terminus of the Proline is further bound to a secondary amine with a cycloalkyl structure attached thereto such as NH(CH₂)₃Ph wherein the nitrogen atom of the amine may be bound to the C-terminus of the Proline. To this end, the resulting chemical structure (as illustrated in Example 19) is the following:

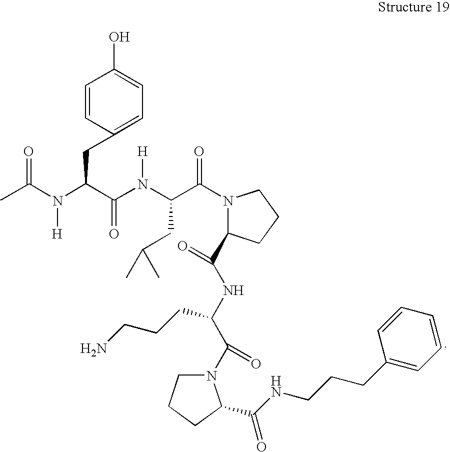

In another non-limiting embodiment, the present invention is represented by the structure of Formula I wherein G is a substituted benzyl group thereby forming Formula III. The X¹, X², and X³ groups are oxygen atoms. The R¹ group is represented by Formula II(a):

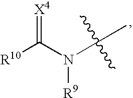

wherein X⁴ is an oxygen atom, R⁹ is a hydrogen atom, and R¹⁰ is a methyl group. The R² and R⁴ groups are hydrogen atoms. The R³ group is a benzoyl group. The R⁵ group is an isobutyl group. The R⁶ and R⁷ groups are each an alkyl group which forms a cyclic ring structure therebetween. Finally, the R⁸ group is an Orn-Pro peptide wherein the N terminus end of the Ornithine is bound to the structure at the R⁸ position of the above structure. The resulting C-terminus of the Proline is further bound to a secondary amine with a cycloalkyl structure attached thereto such as NH(CH₂)₃Ph wherein the nitrogen atom of the amine is bound to the C-terminus of the Proline. To this end, the resulting chemical structure (as illustrated in Example 20) is the following:

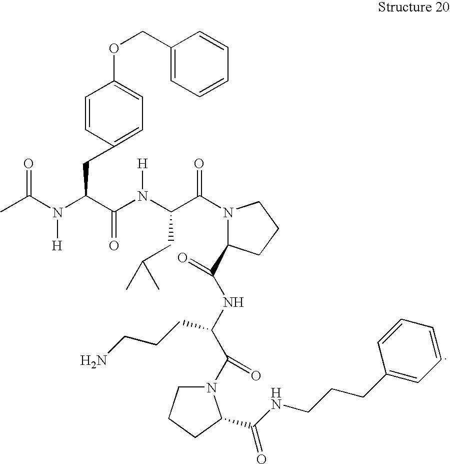

In another non-limiting embodiment, the present invention is represented by the structure of Formula I wherein G is a substituted benzyl group thereby forming Formula III. The X¹, X², and X³ groups are oxygen atoms. The R¹ group is represented by the structure of Formula II(a):

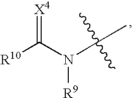

wherein X⁴ is an oxygen atom, R⁹ is a hydrogen atom, and R¹⁰ is a methyl group. The R² and R⁴ groups are hydrogen atoms. The R³ group is a benzoyl group. The R⁵ group is an isobutyl group. The R⁶ and R⁷ groups are each an alkyl groups which form a cyclic ring structure therebetween. Finally, the R⁸ group is an Orn(2-pyrimidine)-Pro peptide wherein the N-terminus end of the peptide is bound to the structure at the R⁸ position of the above structure. The resulting C-terminus of the Proline is further bound to a secondary amine with a cycloalkyl structure attached thereto such as NH(CH₂)₃Ph wherein the nitrogen atom of the amine is bound to the C-terminus of the Proline. To this end, the resulting chemical structure (as illustrated in Example 21) is the following:

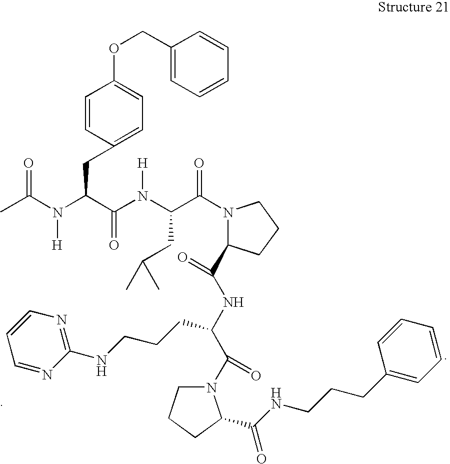

In another non-limiting embodiment, the present invention is represented by the structure of Formula I, wherein G is a sec-butyl group. The X¹, X², and X³ groups are oxygen atoms. The R¹ group is represented by the structure of Formula II(a):

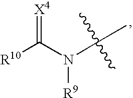

wherein X⁴ is an oxygen atom, R⁹ is a hydrogen atom, and R¹⁰ is a methyl group. The R⁴ group is a hydrogen atom. The R⁵ group is a variant of an Isoleucine side chain wherein one hydrogen of the amine group is substituted with a carboxybenzyl forming the formula (CH₂)₄NHC(O)OCH₂Ph. The R⁶ and R⁷ groups are each an alkyl group which form a cyclic ring structure therebetween. Finally, R⁸ is a hydroxyl group (OH). To this end, the resulting chemical structure (as illustrated in Example 22) is the following:

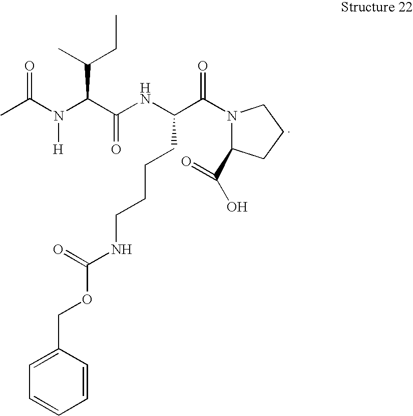

In another non-limiting embodiment, the present invention is represented by the structure of Formula I wherein G is a substituted benzyl group thereby forming Formula III. The X¹, X², and X³ groups are oxygen atoms. The R¹ group is comprised of Formula II(a):

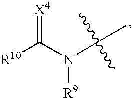

wherein X⁴ is an oxygen atom, R⁹ is a hydrogen atom, and R¹⁰ is a methyl group. The R², R⁴, and R⁶ groups are hydrogen atoms. The R³ group is a hydroxyl group. The R⁵ group is a sec-butyl group. The R⁷ group is a hydroxymethyl group. Finally, R⁸ is a methoxy group (OCH₃). To this end, the resulting chemical structure (as illustrated in Example 23) is the following:

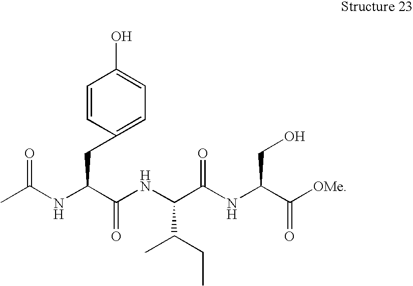

In another non-limiting embodiment, the present invention is represented by the structure of Formula I wherein G is a substituted benzyl group thereby forming Formula III. The X¹, X², and X³ groups are oxygen atoms. The R¹ group is represented by the structure of Formula II(a):

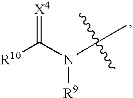

wherein X⁴ is an oxygen atom, R⁹ is a hydrogen atom, and R¹⁰ is a methyl group. The R², R⁴, and R⁶ groups are hydrogen atoms. The R³ group is a hydroxyl group. The R⁵ group is a sec-butyl group. The R⁷ group is a hydroxymethyl group. Finally, R⁸ is a hydroxyl group (OH). To this end, the resulting chemical structure (as illustrated in Example 24) is the following:

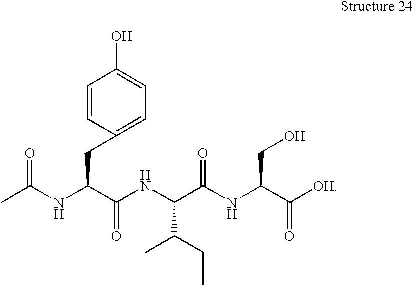

In another non-limiting embodiment, the present invention is represented by the structure of Formula III(I):

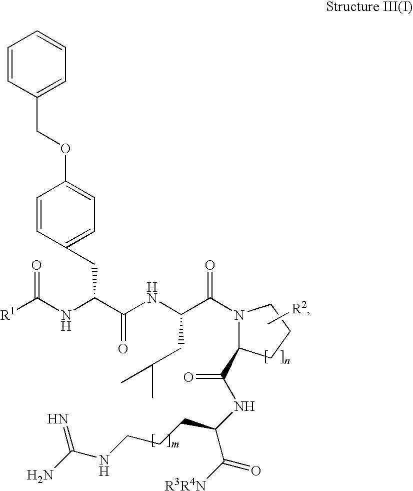

wherein R¹ is selected from a C₃-C₉ alkyl group, R² is selected from hydrogen, hydroxyl, amino, and Structure V:

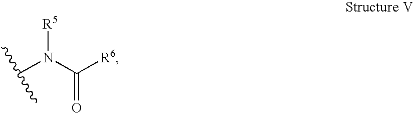

wherein R⁵ is selected from hydrogen, methyl and ethyl, and R⁶ is selected from a C₁-C₄ alkyl group; n in 1, 2 or 3; m is 0, 1 or 2; R³ and R⁴ are independently selected from hydrogen, C₁-C₆ alkyl optionally substituted by a hydroxyl, alkoxy, or acyloxy group, or alternatively R³ and R⁴ together, along with the nitrogen atom to which they are attached, form a five- to seven-membered heterocyclic group,
wherein said heterocyclyl group may optionally contain an additional heteroatom selected from N, S, and O and may optionally be substituted with one or more substituents independently selected from C₁-C₆ alkyl, hydroxyl, C₁-C₆ alkoxy, aryl, and heteroaryl.

In another non-limiting embodiment, the present invention is represented by the structure of formula III(J):

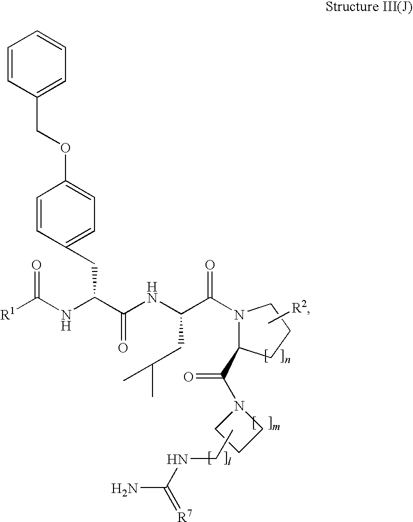

wherein R¹ is selected from a C₃-C₉ alkyl group; R² is selected from hydrogen, hydroxyl, amino, and Structure V

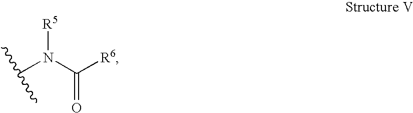

wherein R⁵ is selected from hydrogen, methyl and ethyl, and R⁶ is selected from a C₁-C₄ alkyl group; n is 1, 2 or 3; m is 1, 2, 3 or 4; l is 0, 1 or 2; and R⁷ is selected from O, NH and S.

In another non-limiting embodiment, the present invention is represented by the structure of formula III(K):

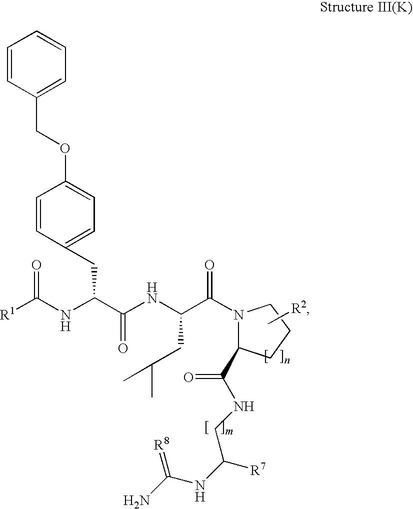

wherein R¹ is selected from a-C₃-C₉ alkyl group; R² is selected from hydrogen, hydroxyl, amino, and Structure V:

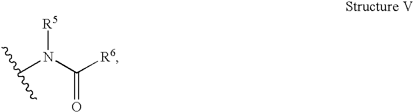

wherein R⁵ is selected from hydrogen, methyl and ethyl, and R⁶ is selected from a C₁-C₄ alkyl group; n is 1, 2 or 3; m is 0, 1, 2, 3 or 4; and R⁷ is selected from alkyl, hydroxyalkyl and alkoxyalkyl; and R⁸ is selected from O, NH and S.

In another embodiment, the present disclosure provides a compound of Formula III(L):

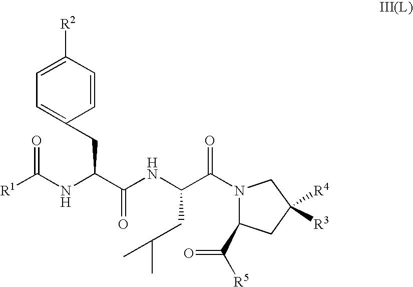

or an enantiomer, a diastereomer, or a pharmaceutically acceptable salt thereof, wherein substituents R¹, R², R³, R⁴, and R⁵ are defined in Table 1.

TABLE 1

| Ex. # | R¹ | R² | R³ | R⁴ | R⁵ |
|---|---|---|---|---|---|
| III(L)-1 | CH₃CH₂CH₂CH₂CH₂CH₂CH₂ | OBzl | H | H | 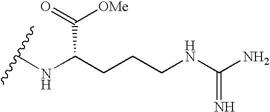 |
| III(L)-2 | CH₃CH₂CH₂CH₂CH₂CH₂CH₂ | OBzl | H | H | 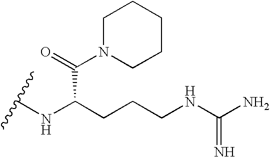 |
| III(L)-3 | 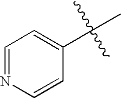 | OBzl | H | H | OMe |
| III(L)-4 | 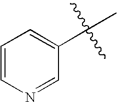 | OBzl | H | H | 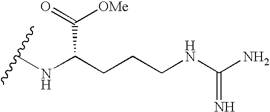 |
| III(L)-5 | 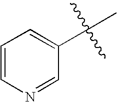 | F | H | H | OMe |
| III(L)-6 | 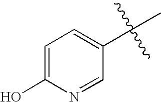 | OBzl | H | H | 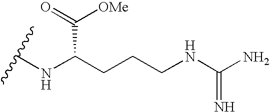 |
| III(L)-7 | CH₃CH₂CH₂CH₂CH₂CH₂CH₂ | OBzl | H | H | 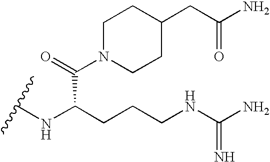 |
| III(L)-8 | CH₃CH₂CH₂CH₂CH₂CH₂CH₂ | OBzl | H | H | 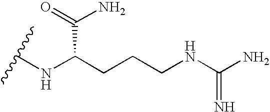 |
| III(L)-9 | CH₃CH₂CH₂CH₂CH₂CH₂CH₂ | OBzl | H | H | 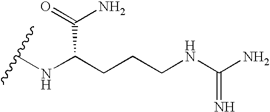 |
| III(L)-10 | CH₃CH₂CH₂CH₂CH₂ | OBzl | H | H | 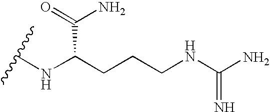 |
| III(L)-11 | CH₃CH₂CH₂ | OBzl | H | H | 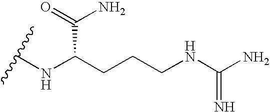 |
| III(L)-12 | Me | OBzl | H | H | 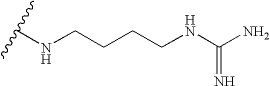 |
| III(L)-13 | Me | OBzl | H | H | 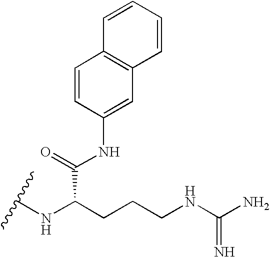 |
| III(L)-14 | Me | OBzl | H | H | 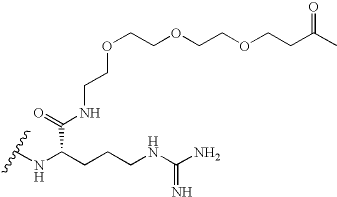 |
| III(L)-15 | 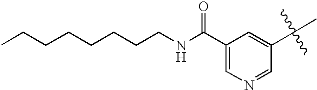 | OBzl | H | H | OMe |
| III(L)-16 | 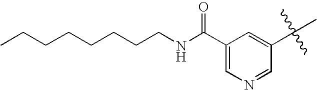 | OBzl | H | H | OH |
| III(L)-17 | 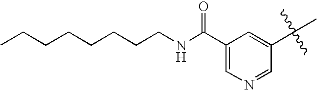 | OH | H | H | 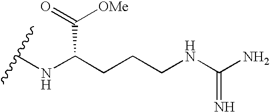 |
| III(L)-18 | 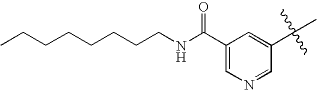 | OBzl | H | H | 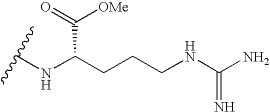 |
| III(L)-19 | CH₃CH₂CH₂CH₂CH₂CH₂CH₂NH | OBzl | H | H | 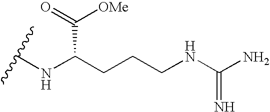 |
| III(L)-20 | CH₃CH₂CH₂CH₂CH₂CH₂CH₂ | F | H | H | 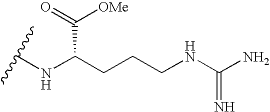 |
| III(L)-21 | CH₃CH₂CH₂CH₂CH₂CH₂CH₂ | OBzl | H | H | 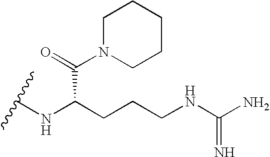 |
| III(L)-22 | CH₃CH₂CH₂CH₂CH₂CH₂CH₂ | OBzl | H | OH | 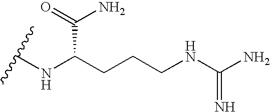 |
| III(L)-23 | CH₃CH₂CH₂CH₂CH₂CH₂CH₂ | OBzl | H | H | 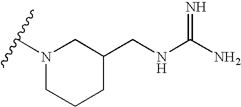 |
| III(L)-24 | CH₃CH₂CH₂CH₂CH₂CH₂CH₂ | OH | H | H | 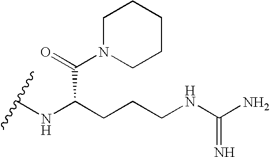 |
| III(L)-25 | 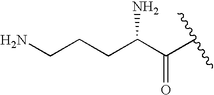 | OH | H | OH | NH₂ |
| III(L)-26 | 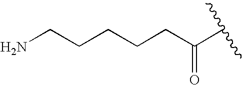 | OBzl | H | OH | 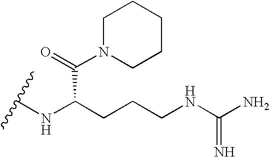 |
| III(L)-27 | CH₃CH₂CH₂CH₂CH₂CH₂CH₂ | OBzl | H | H | 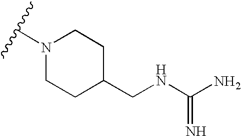 |
| III(L)-28 | CH₃CH₂CH₂CH₂CH₂CH₂CH₂ | OBzl | H | H | 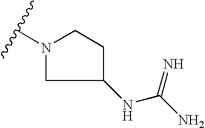 |
| III(L)-29 | CH₃CH₂CH₂CH₂CH₂CH₂CH₂ | OBzl | H | H | 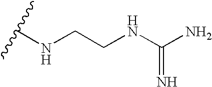 |
| III(L)-30 | CH₃CH₂CH₂CH₂CH₂CH₂CH₂ | OBzl | OH | H | 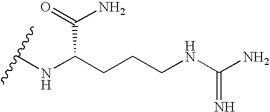 |
| III(L)-31 | CH₃CH₂CH₂CH₂CH₂CH₂CH₂ | OBzl | H | NHAc | 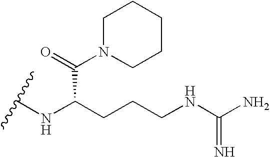 |
| III(L)-32 | CH₃CH₂CH₂CH₂CH₂CH₂CH₂ | OBzl | H | H | 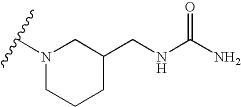 |
| III(L)-33 | 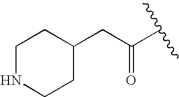 | OBzl | H | OH | 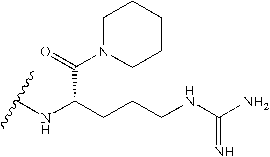 |

In each of the above embodiments where Formula III is illustrated as the exemplary embodiment of the invention, a benzyl or substituted benzyl group is illustrated in the G position of Formula I. However, the variants and combinations of substituent groups of present invention are not limited solely to the structure of Formula III. Rather, each substituent groups and embodiments discussed above are also applicable to all possible combinations of the substituent groups of Formula I. To this end, each embodiment of each substituent group above is applicable to all combinations of both Formula I and Formula III.

Based on the above Formulas, one or more small molecules, or pharmaceutical salts thereof, of the varying combinations of the present invention may be synthesized each with the function of inhibiting the chaperone activity of HSP70 bacterial homologs such as DnaK. Unless otherwise specified, a reference to a particular small molecule includes all such isomeric forms, including all diastereomers, enantiomers, racemic and/or other mixtures thereof. Unless otherwise specified, a reference to a particular compound also includes ionic, salt, solvate (e.g., hydrate), protected forms, and prodrugs thereof. To this end, it may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, the contents of which are incorporated herein by reference.

To test the antibacterial activity of the small molecules of the present invention, it is first necessary to synthesize specific peptides to be studied in vitro. The peptides of the invention may be prepared utilizing any method for peptide synthesis understood in the art, such as those disclosed in Barany et al. The Peptides 2:1-284 1980 and Stewart et al. Solid Phase Peptide Synthesis, Pierce Chemical Co, the contents of which are incorporated herein by reference.

### Synthetic Methods

In one embodiment, the small molecules of the present invention may be prepared using solid-phase peptide synthesis (SPPS). In this method, small solid beads, such as amine resin, are treated with substituent units on which the small molecules can be built. The resultant structure remains covalently attached to the bead until cleaved by a reagent. The structure is thus immobilized on the solid-phase and can be retained during the filtration process.

Due to the amino acid excesses used to ensure complete coupling during each synthesis step, polymerization of amino acids is common. In order to prevent polymerization, protecting groups are used. There are two protecting groups frequently utilized during SPPS, Fmoc and Boc. Fmoc stands for Fluorenyl-methoxy-carbonyl and Boc, or t-Boc, stands for tert-Butyl-oxy-carbonyl. To this end, the present invention contemplates the use of protected natural amino acids and unnatural amino acids. Natural amino acids may be comprised of amino acids which may be found in nature such as Isoleucine, Leucine, Valine, Phenylalanine, Methionine, Cysteine, Alanine, Glycine, Proline, Threonine, Serine, Tyrosine, Tryptophan, Glutamine, Asparagine, Histidine, Glutamic Acid, Aspartic Acid, Lysine and Arginine. Unnatural amino acids are artificial amino acids not naturally found in nature.

SPPS proceeds in a C-terminal to N-terminal fashion by employing repeated cycles of coupling-deprotection. Specifically, the free N-terminal amine of a solid phase attached peptide is coupled to a single N-protected amino acid unit. The unit is then deprotected, revealing a new N-terminal amine to which a further amino acid may be attached. In the present invention, SPPS may be utilized so as for form a peptide of Formula I or Formula III. To this end, 2 to 6 natural or unnatural amino acids may be coupled to the solid phase support resin in the presence of a coupling agent to obtain a peptide. A first substituent group, such as R¹ or R¹⁰, may be added to N terminus of the peptide. Additionally, a second substituent group, such as R⁸, may be added to the C terminus of the peptide. The peptide may then be cleaved from the SPPS. However, the present invention is not limited to this embodiment. Rather the peptide may first be cleaved from the SPPS and the N terminus and C terminus substituent groups added after cleavage. In either case, the resulting peptide is a product of Formula I or Formula III. This product is then purified.

For example, in one embodiment of this invention, a product of Formula I or Formula III can be synthesized by first preparing rink amine resin for automated SPPS. Once prepared, the resin is then subjected to the following automated peptide synthesis: (a) the resin is treated with 20% vv piperidine in DMF for 10 minutes, filtered, then retreated for two 90 minute intervals; (b) the resin is then filtered and washed with DMF; (c) the resin is treated with an amino acid coupling cocktail comprising an Fmoc protected amino acid, PyBOP, HOBt, and DIEA; (d) the resin is mixed for 3 hours, filtered, washed with DMF, and the coupling process repeated twice; (e) the resin is washed with DMF three times to complete the synthesis cycle. This process is repeated until the final product, a compound of Formula I, is obtained. In one embodiment, the Fmoc amino acids employed in this process may be Fmoc-Pro-OH, Fmoc-Arg(Pbf)-OH, Fmoc-Pro-OH, Fmoc-Leu-OH, Fmoc-Tyr(t-Bu)-OH and derivative thereof so as to form the varying substituent groups of Formula I.

Following the completion of SPPS synthesis, the rink amide AM resin is then N-terminal deprotected by treatment with 20% vv piperidine in DMF (3 mL) for 10 minutes, after which the resin is filtered and retreated twice more for 90 minutes. The resin is next filtered and washed with DMF, followed by five washes with 60:40 CH₂Cl₂/DMF and two washes with CH₂Cl₂. The washed resin is then treated with acetic anhydride and pyridine for 1 hour, filtered and washed five additional times with CH₂Cl₂.

Cleavage of the specific embodiment of the invention from the resin is enacted by a three hour treatment with a cleavage cocktail including TFA, water, triethylsilane and thioanisole. The cleavage cocktail is then removed by filtration and retained. The resin is next subjected to a second treatment with cleavage cocktail for 18 hours. The cleavage cocktails, which now include one embodiment of the peptide invention, are combined and dropped into diethyl ether. The mixture next sits for 3 hours at room temperature, and is later stored at −20° C. The resulting precipitates are recovered by centrifugation and washed three times with diethyl ether. Purification of an embodiment of the peptide invention by semi-prep HPLC eluting with a gradient of acetonitrile in water gives a pure final peptide.

In another embodiment of the invention the product of Formula I or Formula III may be prepared by solution based peptide synthesis wherein a plurality of N-protected amino acids is provided wherein each amino acid is protected at its N terminus by a Fmoc protecting group, or the like. The plurality of amino acids may be selected from a group consisting of natural amino acids or derivatives thereof. The amino acids are coupled in a solution so as to form a 2 to 6 amino acid peptide. The protecting groups from each amino acid may then be removed in the peptide resulting in a product of Formula I or Formula III. Additionally, where necessary, additional substituent group, as taught by Formula I or Formula III, may be added to the peptide after removal of the protecting groups. Finally, the product may be purified.

For example, a peptide of Formula I or Formula III can be synthesized by first dissolving proline methyl ester hydrochloride and Boc-Leu-OH, or a derivative thereof, into DMF (20 mL), and adding BOP with stifling. Next, DIEA is added dropwise and the resultant mixture stirred for 48 hours. The reaction mixture is then quenched by addition to water, and extracted with ethyl acetate. The combined organic phases of the extraction are next washed with water, 2N hydrochloric acid, saturated sodium bicarbonate solution and brine, then dried over sodium sulfate. Evaporation of the resultant solvent gives a colorless oil, Boc-Leu-Pro-OMe, or derivative thereof, which can be used in the next step without further purification.

Next, Boc-Leu-Pro-OMe, or derivative thereof, can be used to synthesize Boc-Tyr(Bzl)-Leu-Pro-OMe, or a derivative thereof. First, Boc-Leu-Pro-OMe, or derivative thereof is dissolved in CH₂Cl₂, with TFA added dropwise. After 30 minutes, the volatile components are removed using a rotary evaporator and the residual oil place under vacuum for 2 hours. The resulting glass is then dissolved in DMF (10 mL) and Boc-Tyr(Bzl)-OH, or derivative thereof, with BOP added with stifling. Next, DIEA is added dropwise and the resultant mixture stirred for 72 hours. Later, the reaction mixture is quenched by addition to water, producing a white precipitate that can be recovered by filtration. The resultant solid is then washed with 0.1N sodium hydroxide and water, before drying to give Boc-Tyr-Leu-Pro-OMe, or derivative thereof, as a white solid. One peak by analytical HPLC can be used without further purification.

In a further embodiment, Boc-Tyr(Bzl)-Leu-Pro-OMe, or a derivative thereof, can be used to synthesize Ac-Tyr(Bzl)-Leu-Pro-OMe, or a derivative thereof. First, Boc-Tyr(Bzl)-Leu-Pro-OMe, or a derivative thereof, is dissolved in CH₂Cl₂, with TFA added dropwise. After 45 minutes, the volatile components can be removed using a rotary evaporator and the residual oil placed under vacuum for 2 hours. The resulting glass is then dissolved in pyridine and acetic anhydride, with the resultant mixture stirred for 4 hours. The reaction mixture is then quenched by addition of 2N hydrochloric acid and extracted with ethyl acetate. The organic phase is next washed with 2N hydrochloric acid, water, saturated sodium bicarbonate solution and brine, then dried over sodium sulfate. Evaporation of the solvent resulted in Ac-Tyr(Bzl)-Leu-Pro-OMe, or a derivative thereof.

The examples of each solid phase or solution based peptide synthesis are not intended to be limiting. Rather the small molecules and peptides of the present invention may be synthesized in accordance with, the Examples below or any synthesis method known in the art.

Specific embodiments of the present invention will now be described in the following Examples. The Examples are illustrative only, and are not intended to limit the remainder of the disclosure in any way.

### EXAMPLES

### Example 1

### Structure 1

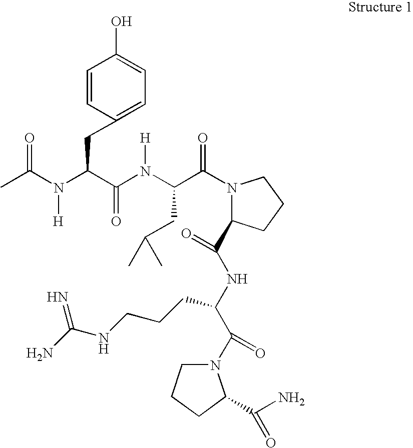

To synthesize Structure 1, rink amine AM resin (100 mg @ 0.68 mmol/g) was swollen and washed with 60:40 CH₂Cl₂/DMF (3 ml) for periods of 10 minutes, and 2×90 minutes. The resin was subsequently washed with DMF (5×3 mL). The swollen resin was then subjected to five iterative cycles of the following automated peptide synthesis:

Resin was treated with 20% vv piperidine in DMF (3 mL) for 10 mins, filtered and retreated for 2×90 minutes. The resin was filtered and washed with DMF (5×3 mL). The resin was treated with an amino acid coupling cocktail comprising an Fmoc protected amino acid (4 eq, 1.07 mL of a 0.255 m DMA solution), PyBOP (4 eq, 1.60 mL of a 0.194 m DMA solution), HOBt (4 eq, 1.07 mL of a 0.255 m DMF solution), and DIEA (8 eq, 1.07 mL of a 0.51 m DMF solution). The resin was mixed for 3 hours, filtered and washed with DMF (3 mL) and the coupling process repeated twice. The resin was washed with DMF (3×3 mL) to complete one synthesis cycle. The Fmoc amino acids employed were consecutively, Fmoc-Pro-OH, Fmoc-Arg(Pbf)-OH, Fmoc-Pro-OH, Fmoc-Leu-OH and Fmoc-Tyr(t-Bu)-OH.

Following completion of the five synthesis cycles, the resin was N-terminal deprotected by treatment with 20% vv piperidine in DMF (3 mL) for 10 minutes, filtered and retreated (2×90 mins). The resin was filtered and washed with DMF (5×3 mL), followed by 60:40 CH₂Cl₂/DMF (5×3 ml) and then CH₂Cl₂ (2×2½ ml). Washed resin was then treated with acetic anhydride (XS, 326 μL), and pyridine (1 mL) for 1 hour, filtered and washed with CH₂Cl₂ (5×3 ml). Cleavage of the peptide from the resin was enacted by treatment with a cleavage cocktail (2½ mL) comprising TFA (90%), water (4%), triethylsilane (4%) and thioanisole (2%) for 3 hr. The cleavage cocktail was removed by filtration and retained. The resin was subjected to a second treatment with cleavage cocktail (4 mL) for 18 hours. The cleavage cocktails were combined and dropped into diethyl ether (40 mL). The mixture was allowed to sit for 3 hours at room temperature, and stored at −20° C. The resulting precipitates were recovered by centrifugation and washed with diethyl ether (3×3 mL). Purification of the crude peptide by semi-prep HPLC eluting with a gradient of acetonitrile in water (both 0.1% TFA) gave the pure final peptide Ac-Tyr-Leu-pro-Arg-Pro-NH₂: 15.2 mg (36%): ¹H NMR (DMSO D₆) δ=9.16 (br s, 1H), 8.07 (d, J=8.0 Hz, 1H), 8.02 (d, J=8.0 Hz, 1H), 7.95 (d, J=9.3 Hz, 1H), 7.43 (t, J=5.9 Hz, 1H), 7.33 (s, 1H), 6.98 (d, J=9.3 Hz, 2H), 6.87 (s, 1H), 6.61 (d, J=10.4 Hz, 2H), 4.47 (m, 2H), 4.42 (m, 1H), 4.33 (dd, J=4.4, 8.4 Hz, 1H), 4.19 (dd, J=4.5, 8.4 Hz, 1H), 3.57 (m, 2H), 3.48 (m, 2H), 3.07 (m, 2H), 2.80 (dd, J=5.6, 14.9 Hz, 1H), 2.56 (dd, J=11.0, 15.0 Hz, 1H), 2.01 (m, 2H), 1.84 (m, 6H), 1.74 (s, 3H), 1.60 (m, 1H), 1.52 (m, 4H), 1.41 (m, 2H), 0.8 (m, 6H) ppm; m/z==686.2 (M+H).

### Example 2

### Structure 2

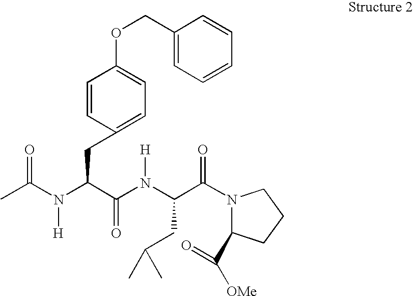

This structure was created by using a step-by-step synthesizing process. First Boc-Leu-Pro-OMe was synthesized using the following method. Proline methyl ester hydrochloride (322 mg, 2 mmol) and Boc-Leu-OH (2 mmol, 498 mg) dissolved in DMF (20 mL) and BOP (2 mmol, 884 mg) added with stifling. DIEA (14 mmol, 2.43 mL) was added dropwise and mixture stirred for 48 h. Reaction mixture quenched by addition to water (200 mL) and extracted with ethyl acetate (4×100 mL). Combined organic phases washed with water (3×150 mL), 2N hydrochloric acid (100 mL), saturated sodium bicarbonate solution (60 mL) and brine (60 mL), then dried over sodium sulfate. Evaporation of solvent gave a colorless oil (596 mg, 87%) which was used without further purification.

Next, Boc-Leu-Pro-OMe (596 mg, 1.75 mmol) was used to synthesize Boc-Tyr(Bzl)-Leu-Pro-OMe. Boc-Leu-Pro-OMe (596 mg, 1.75 mmol) was dissolved in CH₂Cl₂ (8 mL) and TFA (8 mL) added dropwise. After 30 min the volatile components were removed on a rotary evaporator and the residual oil place under vacuum for 2 h. The resulting glass was dissolved in DMF (10 mL) and Boc-Tyr(Bzl)-OH (1 eq, 538 mg), BOP (1 eq, 641 mg) added with stirring. DIEA (6 eq, 1.51 mL) was added dropwise and mixture stirred for 72 h. The reaction mixture was quenched by addition to water (200 mL) producing a white precipitate which was recovered by filtration. The solid was washed with 0.1N sodium hydroxide (2×20 mL) and water (4×20 mL), before drying to give Boc-Tyr-Leu-Pro-OMe as a white solid (769 mg, 74%); one peak by analytical HPLC was used without further purification.

Finally, Boc-Tyr(Bzl)-Leu-Pro-OMe (663 mg, 1.11 mmol) was used to synthesize Structure 2: Ac-Tyr(Bzl)-Leu-Pro-OMe. Boc-Tyr(Bzl)-Leu-Pro-OMe (663 mg, 1.11 mmol) was dissolved in CH₂Cl₂ (5 mL) and TFA (5 mL) added dropwise. After 45 minutes, the volatile components were removed on a rotary evaporator and the residual oil placed under vacuum for 2 hours. The resulting glass was dissolved in pyridine (4 mL) and acetic anhydride (4 eq) added with stifling and mixture for 4 hours. The reaction mixture was then quenched by addition of 2N hydrochloric acid (50 mL) and extracted with ethyl acetate (50 mL). The organic phase was washed with 2N hydrochloric acid (50 mL), water (50 mL), saturated sodium bicarbonate solution (50 mL) and brine (40 mL), then dried over sodium sulfate. Evaporation of solvent gave a white foam of Structure 2 (581 mg, 96%): ¹H NMR (DMSO d₆) δ=8.13 (d, J=8.4 Hz, 1H), 7.97 (d, J=8.4 Hz, 1H), 7.42 (m, 2H), 7.37 (m, 2H), 7.31 (m, 1H), 7.13 (d, J=9.2 Hz, 2H), 6.87 (d, J=9.2 Hz, 2H), 5.04 (s, 2H), 4.52 (m, 1H), 4.45 (m, 1H), 4.29 (dd, J=4.9, 8.4 Hz, 1H), 3.66 (m, 1H), 3.59 (s, 3H), 3.48 (m, 1H), 2.86 (dd, J=9.0, 14.2 Hz, 1H), 2.60 (dd, J=9.1, 14.2 Hz, 1H), 2.15 (m, 1H), 1.90 (m, 2H), 1.80 (m, 1H), 1.73 (s, 3H), 1.62 (m, 1H), 1.43 (m, 2H) 0.89 (m, 6H) ppm; m/z=539 (M+H).

### Example 3

### Structure 3

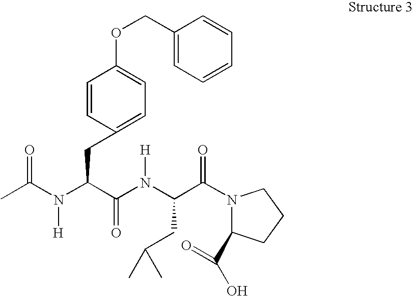

Structure 3 was synthesized using Ac-Tyr(Bzl)-Leu-Pro-OMe (454 mg, 0.85 mmol), which was synthesized using the method explained above. Ac-Tyr(Bzl)-Leu-Pro-OMe was then dissolved in THF (30 mL) and water (20 mL) added dropwise with stifling. Lithium hydroxide monohydrate (12 eq) was added and stifling continued for 4 h. The THF was removed on a rotary evaporator and the remaining aqueous solution diluted to 50 mL with water. 2N Hydrochloric acid (24 eq) was added dropwise and the resulting white solid collected by filtration, and washed with water (3×30 mL). Drying under vacuum gave the desired carboxylic acid (393 mg, 89%): ¹H NMR (DMSO d₆) δ=12.41 (br s, 1H), 8.11 (d, J=8.6 Hz, 1H), 7.96 (d, J=8.6 Hz, 1H), 7.45-7.29 (m, 5H), 7.13 (d, J=8.8 Hz, 2H), 6.87 (d, J=8.8 Hz, 2H), 5.04 (s, 2H), 4.52 (m, 1H), 4.45 (m, 1H), 4.21 (dd, J=5.0, 9.5 Hz, 1H), 3.64 (m, 1H), 3.46 (m, 1H), 2.87 (dd, J=3.9, 13.0 Hz, 1H), 2.61 (dd, J=10.1, 13.7 Hz, 1H), 2.12 (m, 1H), 1.85 (m, 3H), 1.73 (s, 3H), 1.62 (m, 1H), 1.44 (m, 2H) 0.88 (m, 6H) ppm; m/z=522 (M+H).

### Example 4

### Structure 4

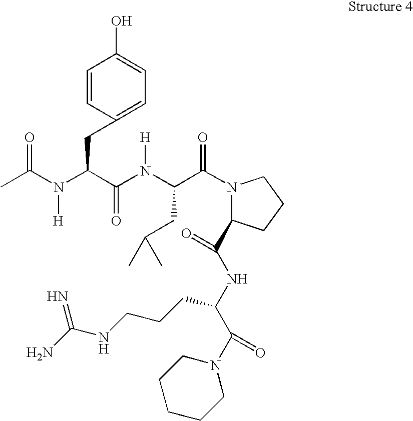

This Structure was created using a step-by-step synthesis process. First, Ac-Tyr(Bzl)-Leu-Pro-OH was used to synthesize Ac-Tyr(Bzl)-Leu-Pro-Arg(Pbf)-OMe. Ac-Tyr(Bzl)-Leu-Pro-OH (296 mg, 0.57 mmol) was dissolved in DMF (5 mL) and H-Arg(Pbf)-OMe (1 eq) and BOP (1 eq) were added with stifling. DIEA (6 eq) was then added, and the reaction mixture stirred for 20 hours. The resultant mixture was quenched by addition to water (150 mL), and brine (5 mL). Stirring was continued for 2 hours to give a fine white precipitate. 1N Potassium hydroxide (5 mL) was added and stirring continued for 5 minutes. The solid was collected by filtration, and washed with 0.025N potassium hydroxide (30 mL), and water (2×30 mL). Drying under vacuum gave the desired ester (441 mg, 79%). The sample was used in the next step without further purification.

Next Ac-Tyr(Bzl)-Leu-Pro-Arg(Pbf)-OMe OMe (393 mg, 0.40 mmol) was dissolved in THF (15 mL). Water (20 mL) was then added dropwise with stirring. Lithium hydroxide monohydrate (12 eq) was added and stirring continued for 4 hours. THF was next removed on a rotary evaporator and the remaining aqueous solution diluted to 50 mL with water. 2N Hydrochloric acid (24 eq) was added dropwise and the resulting white solid collected by filtration, and washed with water (3×10 mL). Drying under vacuum gave the desired carboxylic acid: Ac-Tyr(Bzl)-Leu-Pro-Arg(Pbf)-OH (321 mg, 83%). The resultant sample of was used in the next step without further purification.

Next, Ac-Tyr(Bzl)-Leu-Pro-Arg(Pbf)-OH (34.2 mg, 0.035 mmol) was dissolved in DMF (0.5 mL). Piperidine (1 eq) and BOP (1 eq) were added with stirring. DIEA (6 eq) was added, and the reaction mixture stirred for 20 hours. The resultant mixture was quenched by the addition of 0.1N potassium hydroxide (10 mL). Stirring was continued for 1 hour to give a fine white precipitate. The solid was collected by filtration, and washed with water (5×2 mL). Drying under vacuum gave the desired piperidinyl amine: Ac-Tyr(Bzl)-Leu-Pro-Arg(Pbf)-N(CH₂)₅ (38.2 mg, 100%). The sample was used without further purification.

Next, Ac-Tyr(Bzl)-Leu-Pro-Arg(Pbf)-N(CH₂)₅ (11 mg, 0.01 mmol) was dissolved in methanol (1 mL) and 5% palladium on charcoal (9.5 mg, 25 mol %). The atmosphere was replaced by a hydrogen atmosphere at standard pressure and the reaction mixture stirred for 2 hours. The reaction mixture was filtered through a plug of silica/alumina catalyst support and evaporated to give the deprotected tyrosine analog as a foam: Ac-Tyr-Leu-Pro-Arg(Pbf)-N(CH₂)₅ (7.7 mg, 77%), which was used without further purification.

Finally, Ac-Tyr-Leu-Pro-Arg(Pbf)-N(CH₂)₅ (7.7 mg, 0.008 mmol) was dissolved in TFA (0.5 mL) and stirred for 2 hours. The sample was then evaporated to dryness and purified by semi-prep HPLC to give the desired fully protected tetrapeptide amine, Ac-Tyr-Leu-Pro-Arg-N(CH₂)₅.TFA, as a white solid (4.8 mg, 81%): ¹H NMR (deuteromethanol) δ=8.24 (d, 1H), 7.97 (d, 1H), 7.03 (m, 2H), 6.66 (m, 2H), 4.78 (m, 1H), 4.66 (m, 1H), 4.55 (m, 1H), 4.37 (m, 1H), 3.70 (m, 1H), 3.54 (m, 3H), 3.44 (m, 2H), 3.21 (m, 2H), 2.99 (dd, 1H), 2.76 (dd, 1H), 2.20 (m, 1H), 2.07 (m, 1H), 1.97 (m, 1H), 1.90 (s, 3H), 1.71-1.49 (m, 14H), 0.94 (m, 6H) ppm; m/z=657.4 (M+H).

### Example 5

### Structure 5

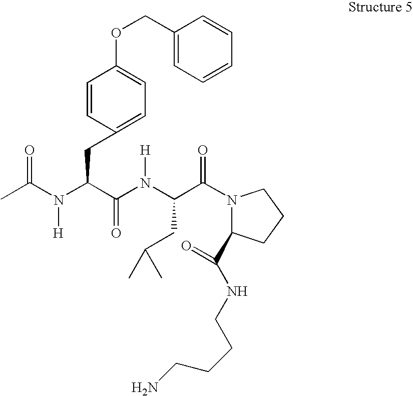

This Structure was created using a step-by-step synthesis process. First, Ac-Tyr(Bzl)-Leu-Pro-OH (52.3 mg, 0.1 mmol) was dissolved in DMF (1 mL). H₂N(CH₂)₄NHBoc (1 eq) and BOP (1 eq) were added with stirring. DIEA (6 eq) was added, and the reaction mixture stirred for 66 hours. The resultant mixture was quenched by addition of 0.1N potassium hydroxide (20 mL). Stirring was continued for 4 hours to give a pale cream precipitate. The solid was collected by filtration and washed with water (4×5 mL). Drying under vacuum gave the desired peptide amine, Ac-Tyr(Bzl)-Leu-Pro-NH(CH₂)₄NHBoc (52 mg, 75%). The sample was used without further purification.

Next, Ac-Tyr(Bzl)-Leu-Pro-NH(CH₂)₄NHBoc (13.9 mg, 0.02 mmol) was dissolved in CH₂Cl₂ (1 mL) and TFA (1 mL) added dropwise. After 45 min the volatile components were removed on a rotary evaporator and the residual oil place under vacuum for 2 h. The resultant oil was washed with ethyl acetate (2 mL) and then dissolved in water (5 mL) and lyophilized to give a white powder of the desired product, Ac-Tyr(Bzl)-Leu-Pro-NH(CH₂)₄NH₂.TFA (11 mg, 78%): ¹H NMR (DMSO d₆) δ=8.06 (d, J=12.0 Hz, 1H), 7.98 (d, J=8.4 Hz, 1H), 7.83 (t, J=5.9 Hz, 1H), 7.61 (br s, 3H) 7.44-7.29 (m, 5H), 7.12 (d, J=8.4 Hz, 2H), 6.88 (d, J=8.4 Hz, 2H), 5.03 (s, 2H), 4.51 (m, 1H), 4.45 (m, 1H), 4.21 (dd, J=3.9, 7.2 Hz, 1H), 3.59 (m, 1H), 3.46 (m, 1H), 3.07 (m, 1H), 2.99 (m, 1H), 2.86 (dd, J=3.9, 13.6 Hz, 1H), 2.76 (m, 2H), 2.60 (dd, J=9.2, 14.0 Hz, 1H), 2.00 (m, 1H), 1.90 (m, 1H), 1.80 (m, 1H), 1.73 (s, 3H), 1.72 (m, 1H), 1.62 (m, 1H), 1.53-1.37 (m, 2H), 1.24 (m, 2H), 0.89 (m, 6H) ppm; m/z=593.4 (M+H).

### Example 6

### Structure 6

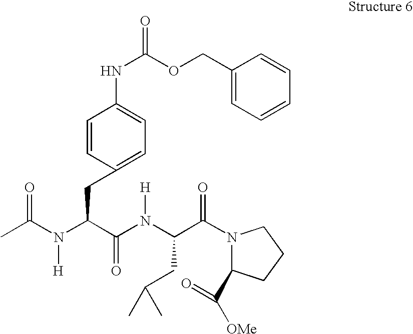

This Structure was synthesized using a step-by-step process. First, Boc-Leu-Pro-OMe (342 mg, 1.00 mmol) was dissolved in CH₂Cl₂ (5 mL) and TFA (5 mL) added dropwise. After 45 minutes, the volatile components were removed using a rotary evaporator, and the residual oil placed under vacuum for 2 hours. The resulting glass was dissolved in DMF (10 mL) and Boc-4-(NHCbz)Phe-OH (1 eq, 414 mg). BOP (1 eq, 442 mg) was added with stirring. DIEA (6 eq, 1.04 mL) was added dropwise and the mixture stirred for 48 hours. The reaction mixture was quenched by addition to water (200 mL) producing a white emulsion. After stirring for 2 hours, sodium chloride (˜5 g) was added to the emulsion and stirring continued for 1 hour. The mixture was made basic with saturated aqueous sodium bicarbonate (5 mL) and stirred an additional 15 minutes. The resulting off-white precipitate was recovered by filtration. The solid was washed with water (5×20 mL), before drying to give Boc-4-(NHCbz)Phe-Leu-Pro-OMe as an off-white solid (554 mg, 87%). One peak purified by analytical HPLC was used without further purification.

Next, Boc-4-(NHCbz)Phe-Leu-Pro-OMe (319 mg, 0.5 mmol) was dissolved in CH₂Cl₂ (5 mL), with TFA (5 mL) added dropwise. After 45 minutes, the volatile components were removed on a rotary evaporator and the residual oil placed under vacuum for 18 hours. The resulting glass was dissolved in pyridine (2 mL) and acetic anhydride (4 eq) added with stifling. The mixture was then stirred for 4 hours. The reaction mixture was quenched by addition to water (100 mL) and extracted with ethyl acetate (100 mL). The organic phase was separated and washed with water (100 mL), 2N hydrochloric acid (2×75 mL), saturated sodium bicarbonate solution (2×50 mL), water (75 mL), and brine (50 mL), then dried over sodium sulfate. Evaporation of solvent gave a tan foam of the desired product, Ac-4-(NHCbz)Phe-Leu-Pro-OMe (259 mg, 89%): ¹H NMR (DMSO d₆) δ=9.68 (s, 1H), 8.13 (d, J=8.2 Hz, 1H), 7.94 (d, J=9.1 Hz, 1H), 7.44-7.29 (m, 7H), 7.09 (d, J=8.6 Hz, 2H), 5.13 (s, 2H), 4.53 (m, 1H), 4.47 (m, 1H), 4.28 (dd, J=5.3, 9.3 Hz, 1H), 3.63 (m, 1H), 3.59 (m, 3H), 3.47 (m, 1H), 2.86 (dd, J=4.5, 14.4 Hz, 1H), 2.62 (dd, J=9.2, 13.2 Hz, 1H), 2.14 (m, 1H), 1.89 (m, 2H), 1.79 (m, 1H), 1.74 (s, 3H), 1.61 (m, 1H), 1.43 (m, 2H) 0.88 (m, 6H) ppm; m/z=581.4 (M+H).

### Example 7

### Structure 7

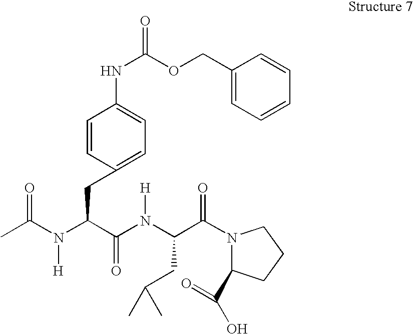

First, Ac-4-(NHCbz)Phe-Leu-Pro-OMe (58 mg, 0.10 mmol) was dissolved in THF (4 mL), with water (4 mL) added dropwise with stirring. Lithium hydroxide monohydrate (12 eq) was added and stirring continued for 3 hours. The THF was removed on a rotary evaporator and the remaining aqueous solution diluted to 15 mL with water. 2N Hydrochloric acid (24 eq) was added dropwise and the resulting white solid collected by filtration, and washed with water (3×10 mL). Drying under vacuum gave the desired carboxylic acid, Ac-4-(NHCbz)Phe-Leu-Pro-OH (46 mg, 81%): ¹H NMR (DMSO d₆) δ=12.4 (br s, 1H), 9.65 (s, 1H), 8.09 (d, J=8.8 Hz, 1H), 7.91 (d, J=8.7 Hz, 1H), 7.42-7.25 (m, 7H), 7.07 (d, J=8.8 Hz, 2H), 5.10 (s, 2H), 4.50 (m, 1H), 4.44 (m, 1H), 4.18 (dd, J=5.5, 7.8 Hz, 1H), 3.60 (m, 1H), 3.43 (m, 1H), 2.84 (dd, J=3.2, 12.0 Hz, 1H), 2.60 (dd, J=8.7, 12.8 Hz, 1H), 2.10 (m, 1H), 1.84 (m, 2H), 1.71 (s, 3H), 1.59 (m, 1H), 1.41 (m, 2H) 1.24 (m, 1H), 0.85 (m, 6H) ppm; m/z=567 (M+H).

### Example 8

### Structure 8

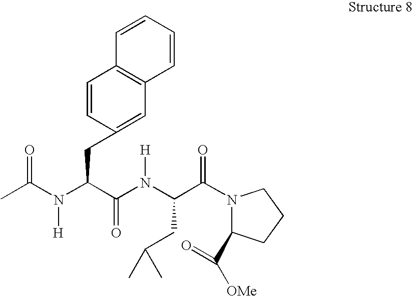

The desired methyl ester was prepared in prepared in an analogous manner to Example 6—Structure 6 CHP-205 (040207_{—}2.1) employing Boc-2-Nal-OH in place of Boc-4-(NHCbz)Phe-OH: ¹H NMR (DMSO d₆) δ=8.18 (d, J=7.9 Hz, 1H), 8.04 (d, J=8.0 Hz, 1H), 7.82 (m, 1H), 7.77 (d, J=8.0 Hz, 1H), 7.68 (s, 1H), 7.49-7.37 (m, 4H), 4.62 (m, 1H), 4.50 (dd, J=8.0, 15.2 Hz, 1H), 4.21 (dd, J=5.6, 8.4 Hz, 1H), 3.59 (m, 1H), 3.57 (s, 3H), 3.43 (m, 1H), 3.08 (dd, J=5.2, 14.4 Hz, 1H), 2.83 (dd, J=8.6, 13.2 Hz, 1H), 2.05 (m, 1H), 1.85 (m, 1H), 1.76 (m, 2H), 1.70 (s, 3H), 1.61 (m, 1H), 1.42 (m, 2H) 0.87 (m, 6H) ppm; m/z=482.2 (M+H).

### Example 9

### Structure 9

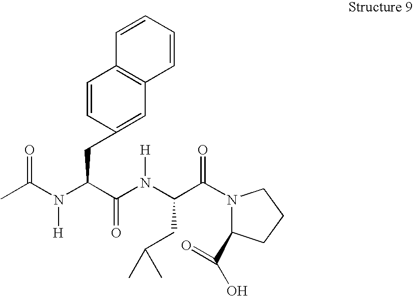

The desired carboxylic acid was prepared in an analogous manner to Example 7—Structure 7 CHP-203 (042407_{—}1.1) employing Ac-2-Nal-Leu-Pro-OMe in place of Ac-4-(NHCbz)Phe-Leu-Pro-OMe: ¹H NMR (DMSO d₆) δ=12.4 (br s, 1H), 8.18 (d, 1H), 8.04 (d, 1H), 7.82-7.74 (m, 2H), 7.66 (s, 1H), 7.46-7.37 (m, 4H), 4.61 (m, 1H), 4.50 (m, 1H), 4.12 (m, 1H), 3.58 (m, 1H), 3.44 (m, 1H), 3.11 (m, 1H), 2.82 (m, 1H), 2.03 (m, 1H), 1.81 (m, 1H), 1.77 (m, 2H), 1.70 (s, 3H), 1.59 (m, 1H), 1.39 (m, 2H) 0.88 (m, 6H) ppm; m/z=468.3 (M+H).

### Example 10

### Structure 10

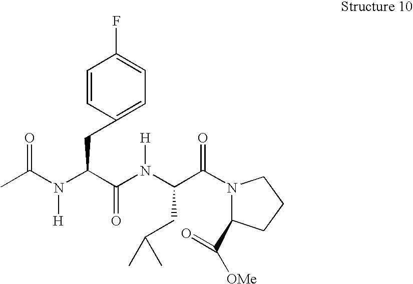

The desired methyl ester was prepared in prepared in an analogous manner to CHP-205 (040207_{—}2.1) employing Boc-(4-F)Phe-OH in place of Boc-4-(NHCbz)Phe-OH: ¹H NMR (DMSO d₆) δ=8.14 (d, J=8.2 Hz, 1H), 7.98 (d, J=8.6 Hz, 1H), 7.22 (m, 2H), 7.03 (m, 2H), 4.48 (m, 2H), 4.26 (dd, J=5.2, 10.0 Hz, 1H), 3.64 (m, 1H), 3.57 (s, 3H), 3.46 (m, 1H), 2.89 (dd, J=3.9, 13.2 Hz, 1H), 2.63 (dd, J=10.4, 13.6 Hz, 1H), 2.13 (m, 1H), 1.88 (m, 2H), 1.77 (m, 1H), 1.70 (s, 3H), 1.59 (m, 1H), 1.41 (m, 2H) 0.86 (m, 6H) ppm; m/z=450.2 (M+H).

### Example 11

### Structure 11

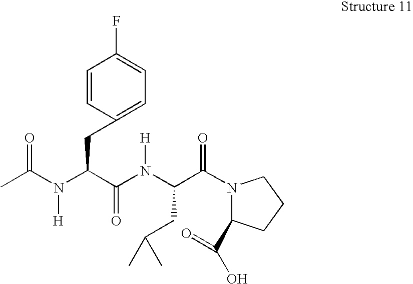

The desired carboxylic acid was prepared in prepared in an analogous manner to Example 7—Structure 7 CHP-203 (042407_{—}1.1) employing Ac(4-F)Phe-Leu-Pro-OMe in place of Ac-4-(NHCbz)Phe-Leu-Pro-OMe: ¹H NMR (DMSO d₆) δ=12.43 (br s, 1H), 8.16 (d, J=8.1 Hz, 1H), 8.01 (d, J=8.1 Hz, 1H), 7.24 (m, 2H), 7.06 (m, 2H), 4.51 (m, 2H), 4.21 (dd, J=5.5, 9.1 Hz, 1H), 3.64 (m, 1H), 3.47 (m, 1H), 2.92 (dd, J=5.0, 14.6 Hz, 1H), 2.66 (dd, J=10.2, 13.2 Hz, 1H), 2.12 (m, 1H), 1.89 (m, 2H), 1.82 (m, 1H), 1.73 (s, 3H), 1.62 (m, 1H), 1.44 (m, 2H) 0.88 (m, 6H) ppm; m/z=436.3 (M+H).

### Example 12

### Structure 12

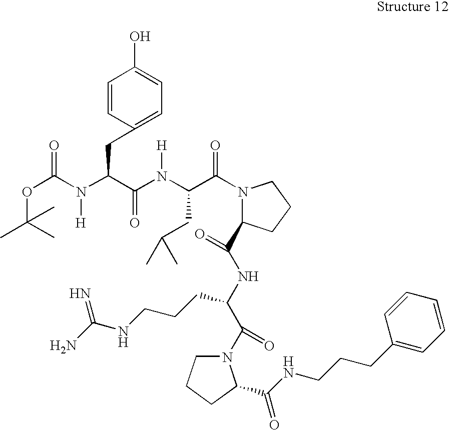

This Structure was created using a step-by-step synthesis process. First, Boc-Arg(bis-Cbz)-OH (2715 mg, 5 mmol) was dissolved in DMF (50 mL). Proline methyl ester hydrochloride (1 eq) and BOP (1 eq) were added with stirring. DIEA (7 eq) was added, and the reaction mixture stirred for 72 hours. The resultant mixture was quenched by addition to 0.1N potassium hydroxide (600 mL) and extracted with ethyl ether/ethyl acetate (2×200 mL). The combined organic phases were washed with water (4×75 mL) and brine (50 mL), then dried over sodium sulfate. Evaporation of solvent gave a colorless foam, BocArg(bis-Cbz)-Pro-OMe (2780 mg, 85%) which was used without further purification.

Next, Boc-Arg(bis-Cbz)-Pro-OMe (654 mg, 1 mmol) was dissolved in CH₂Cl₂ (5 mL) and TFA (5 mL), which was added dropwise. After 45 minutes, the volatile components were removed on a rotary evaporator and the residual oil placed under vacuum for 2 hours. The resulting glass was dissolved in DMF (10 mL). Boc-Pro-OH (1 eq) and BOP (1 eq) were added with stifling. DIEA (6 eq) was added, and the reaction mixture stirred for 120 hours. The resultant mixture was quenched by addition to water (200 mL) and extracted with ethyl acetate (4×50 mL). The combined organic phases were washed with water (3×75 mL), 1N hydrochloric acid (50 mL), 0.5N potassium hydroxide (50 mL), water (50 mL) and brine (50 mL), and then dried over sodium sulfate. Evaporation of the solvent gave a tan foam, Boc-Pro-Arg(bis-Cbz)-Pro-OMe (683 mg, 91%) which was used without further purification.

Next, Boc-Pro-Arg(bis-Cbz)-Pro-OMe (683 mg, 0.91 mmol) was dissolved in CH₂Cl₂ (5 mL) and TFA (5 mL) added dropwise. After 45 minutes, the volatile components were removed using a rotary evaporator and the residual oil placed under vacuum for 2 hours. The resulting glass was dissolved in DMF (9 mL). Boc-Pro-OH (1 eq) and BOP (1 eq) were added with stirring. DIEA (6 eq) was added, and the reaction mixture stirred for 120 hours. The resultant mixture was quenched by addition to water (200 mL) and extracted with ethyl acetate (3×50 mL). The combined organic phases were washed with water (5×75 mL) and brine (50 mL), and then dried over sodium sulfate. Evaporation of solvent gave a cream foam, Boc-Leu-Pro-Arg(bis-Cbz)-Pro-OMe (750 mg, 95%) which was used without further purification.

Next, Boc-Leu-Pro-Arg(bis-Cbz)-Pro-OMe (648 mg, 0.75 mmol) was dissolved in CH₂Cl₂ (4 mL), with TFA (4 mL) added dropwise. After 45 minutes, the volatile components were removed on a rotary evaporator and the residual oil placed under vacuum for 2 hours. The resulting glass was dissolved in DMF (8 mL). Boc-Pro-OH (1 eq) and BOP (1 eq) were added with stifling. DIEA (6 eq) was added, and the reaction mixture stirred for 120 hours. The resultant mixture was quenched by addition to water (200 mL) leading to a pale cream precipitate. Stirring was continued for 60 minutes. 1N Potassium hydroxide (15 mL) was added and stifling continued for 15 minutes. The precipitate was collected by filtration, washed with water (4×40 mL) and dried under vacuum to give the desired product as a cream solid, Boc-Tyr(Bzl)-Leu-Pro-Arg(bis-Cbz)-Pro-OMe (839 mg, 100%), which was used without further purification.

Next, Boc-Leu-Pro-Arg(bis-Cbz)-Pro-OMe (559 mg, 0.5 mmol) was dissolved in THF (21 mL) and water (14 mL), which was added dropwise with stifling. Lithium hydroxide monohydrate (12 eq) was added and stifling continued for 68 hours. The THF was removed using a rotary evaporator and the remaining aqueous solution diluted to 100 mL with water. The resulting mixture was extracted with ethyl acetate (75 mL) and to the aqueous mixture, 2N Hydrochloric acid (24 eq) was added dropwise. The resulting white solid was collected by filtration, and washed with water (5×20 mL). Drying under vacuum gave the desired carboxylic acid as a pale cream solid, Boc-Tyr(Bzl)-Leu-Pro-Arg(Cbz)-Pro-OH (385 mg, 70%).

Boc-Tyr(Bzl)-Leu-Pro-Arg(Cbz)-Pro-OH (36.4 mg, 0.033 mmol) was dissolved in DMF (0.5 mL). 3-phenylpropylamine (1 eq) and BOP (1 eq) were added with stirring. DIEA (6 eq) was added, and the reaction mixture stirred for 42 h. The resultant mixture was quenched by addition to 0.1N potassium hydroxide (10 mL) and brine (1 mL). Stifling was continued for 2 h to give a fine white precipitate. The solid was collected by filtration, and washed with water (3×4 mL). Drying under vacuum gave the desired amine, Boc-Tyr(Bzl)-Leu-Pro-Arg(Cbz)-Pro-NH(CH₂)₃Ph (30 mg, 75%). The sample was used without further purification.

Next, Boc-Tyr(Bzl)-Leu-Pro-Arg(Cbz)-Pro-NH(CH₂)₃Ph (36.4 mg, 0.033 mmol) was dissolved in methanol (2 mL) and 5% palladium on charcoal (19 mg, 25 mol %) was added. The atmosphere was replaced by a hydrogen atmosphere at standard pressure and the reaction mixture stirred for 72 hours. The reaction mixture was filtered through a plug of silica/alumina catalyst support and evaporated to give the crude deprotected peptide. Crude product was purified by reverse phase semi preparative HPLC to give the desired product as a white solid, Boc-Tyr-Leu-Pro-Arg-Pro-NH(CH₂)₃Ph (7.3 mg, 39%): ¹H NMR (deuteromethanol) δ=8.19 (d, 1H), 8.13 (m, 2H), 7.91 (d, 1H), 7.21 (m, 5H), 7.01 (m, 2H), 6.67 (m, 2H), 4.78 (m, 2H), 4.65 (m, 1H), 4.37 (m, 1H), 4.23 (m, 1H), 3.79 (m, 1H), 3.71 (m, 1H), 3.62 (m, 2H), 3.57 (m, 1H), 3.21 (m, 2H), 2.95 (dd, 1H), 2.72 (dd, 1H), 2.64 (m, 2H), 2.21 (m, 2H), 2.12-1.85 (m, 7H), 1.83 (m, 2H), 1.73 (m, 4H), 1.54 (m, 2H), 1.33 (m, 9H), 0.94 (m, 6H) ppm; m/z=862 (M+H)

### Example 13

### Structure 13

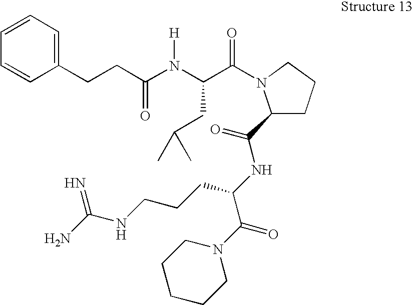

This Structure was created using a step-by-step synthesis process. First, Boc-Arg(bis-Cbz)-OH (271.3 mg, 0.5 mmol) was dissolved in DMF (5 mL). Piperidine (1 eq) and BOP (1 eq) were added with stifling. DIEA (6 eq) was added, and the reaction mixture stirred for 44 hours. The resultant mixture was quenched by addition to water (100 mL) and extracted with ethyl acetate (2×100 mL). The combined organic phases were then washed with 0.1N potassium hydroxide (100 mL), water (100 mL), 0.2N hydrochloric acid (100 mL), water (4×100 mL) and brine (50 mL), and dried over sodium sulfate. Evaporation of the solvent gave a clear oily solid, Boc-Arg(bis-Cbz)-N(CH₂)₅ (296 mg, 97%) which was used without further purification.

Next, Boc-Arg(bis-Cbz)-N(CH₂)₅ (296 mg, 0.485 mmol) was dissolved in CH₂Cl₂ (2.5 mL) and TFA (2.5 mL) added dropwise. After 45 minutes, the volatile components were removed on a rotary evaporator and the residual oil placed under vacuum for 2 hours. The resulting glass was dissolved in DMF (5 mL). Boc-Pro-OH (1 eq) and BOP (1 eq) were added with stifling. DIEA (6 eq) was then added, and the reaction mixture stirred for 96 hours. The resultant mixture was quenched by the addition of water (100 mL), leading to a tan emulsion, and extracted with ethyl acetate (2×50 mL). The combined organic phases were washed with water (5×50 mL) and brine (50 mL), and then dried over sodium sulfate. Evaporation of solvent gave a brown sticky foam, Boc-Pro-Arg(bis-Cbz)-N(CH₂)₅ (309 mg, 90%) which was used in the next step without further purification.

Next, Boc-Pro-Arg(bis-Cbz)-N(CH₂)₅ (309 mg, 0.44 mmol) was dissolved in CH₂Cl₂ (2.5 mL) and TFA (2.5 mL) added dropwise. After 45 minutes, the volatile components were removed using a rotary evaporator and the residual oil placed under vacuum for 2 hours. The resulting glass was dissolved in DMF (5 mL). Boc-Pro-OH (1 eq) and BOP (1 eq) were added with stirring. DIEA (6 eq) was added, and the reaction mixture stirred for 41 h. The resultant mixture was quenched by addition to 0.1N potassium hydroxide (150 mL). Stifling was continued for 4 hours to give an orange precipitate. The solid was collected by filtration, and washed with water (3×10 mL). Drying under vacuum gave the desired amine, Boc-Leu-Pro-Arg(bis-Cbz)-N(CH₂)₅ (301 mg, 84%). The sample was used without further purification.

Next, Boc-Leu-Pro-Arg(bis-Cbz)-N(CH₂)₅ (102.5 mg, 0.125 mmol) was dissolved in CH₂Cl₂ (1 mL) and TFA (1 mL) added dropwise. After 45 minutes, the volatile components were removed on a rotary evaporator and the residual oil placed under vacuum for 2 hours. The resulting glass was dissolved in DMF (1.25 mL). Hydrocinnamic acid (1 eq) and BOP (1 eq) were added with stirring. DIEA (6 eq) was then added, and the reaction mixture stirred for 20 hours. The resultant mixture was quenched by addition to 0.1N potassium hydroxide (30 mL). The resultant brown oily residue was diluted with water (70 mL) and extracted with ethyl acetate (2×50 mL). The combined organic extracts were washed with water (4×50 mL) and brine (930 mL), and dried over sodium sulfate. Evaporation of the solvent gave the desired amine, Ph(CH₂)₂C(O)-Leu-Pro-Arg(bis-Cbz)-N(CH₂)₅, as an orange/tan foam (81 mg, 76%). The sample was used without further purification.

In the final phase of this synthesis, Ph(CH₂)₂C(O)-Leu-Pro-Arg(bis-Cbz)-N(CH₂)₅ (42.6 mg, 0.05 mmol) was dissolved in methanol (2 mL) and 5% palladium on charcoal (445 mg, 25 mol %). TFA (2 eq) was added. The atmosphere was replaced by a hydrogen atmosphere at standard pressure and the reaction mixture stirred for 20 hours. The reaction mixture was filtered through a plug of silica/alumina catalyst support and evaporated to give the crude deprotected peptide, Ph(CH₂)₂C(O)-Leu-Pro-Arg-N(CH₂)₅. Crude product was purified by reverse phase semi preparative HPLC to give the desired product; TFA salt as a white solid (15 mg, 43%): ¹H NMR (deuteromethanol) δ=8.22 (d, 1H), 8.03 (d, 1H), 7.23 (m, 2H), 7.17 (m, 3H), 4.84 (m, 1H), 4.59 (m, 1H), 4.39 (dd, 1H), 3.83 (m, 1H), 3.59 (m, 2H), 3.51 (m, 2H), 3.47 (m, 2H), 3.20 (m, 2H), 2.90 (t, 2H), 2.53 (m, 2H), 2.19 (m, 1H), 2.04 (m, 1H), 1.97 (m, 1H), 1.90 (m, 1H), 1.77 (m, 1H), 1.69-1.47 (m, 12H), 0.91 (d, 3H), 0.88 (d, 3H) ppm; m/z=584.3 (M+H).

### Example 14

### Structure 14

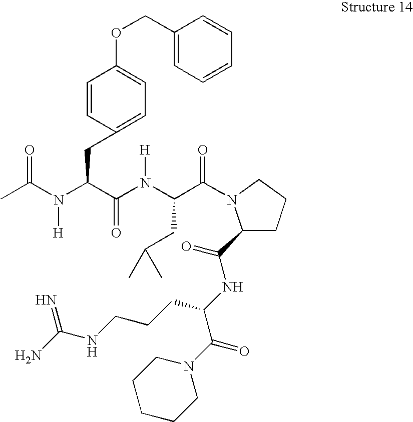

Ac-Tyr(Bzl)-Leu-Pro-Arg(Pbf)-N(CH₂)₅ (11 mg, 0.01 mmol) was dissolved in TFA (0.475 mL) and water (0.025 mL) and stirred for 3 h. Evaporated to dryness and purified by semi-prep HPLC gave the desired fully protected tetrapeptide amine as a white solid (3.3 mg, 36%): ¹H NMR (CD₃OD) δ=7.20 (m, 7H), 7.12 (m, 2H), 6.91 (s, 1H), 6.88 (m, 1H), 6.60 (d, J=12.0 Hz, 1H), 5.47 (s, 2H), 4.84 (m, 1H), 4.65 (m, 1H), 4.51 (m, 1H), 4.36 (m, 1H), 3.70-3.40 (m, 6H), 3.18 (m, 2H), 2.98 (m, 1H), 2.71 (m, 1H), 2.14 (m, 1H), 2.03 (m, 1H), 1.94 (m, 2H), 1.84 (s, 3H), 1.75-1.55 (m, 13H), 0.94 (m, 6H) ppm; m/z=747.4 (M+H).

### Example 15

### Structure 15

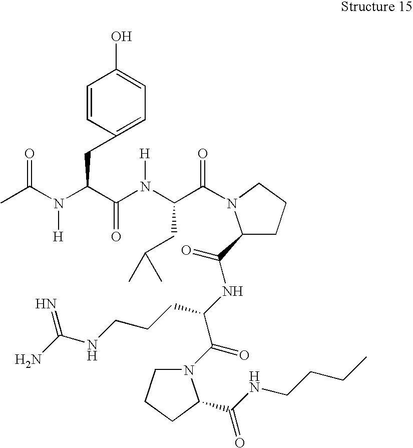

This Structure was created using a step-by-step synthesis process. First, Ac-Tyr(Bzl)-Leu-Pro-Arg(Pbf)-OH (207 mg, 0.21 mmol) was dissolved in DMF (2 mL). Proline methyl ester hydrochloride (1 eq) and BOP (1 eq) were added with stifling. DIEA (6 eq) was added, and the reaction mixture stirred for 20 hours. The resultant mixture was quenched by addition to water (35 mL). Stirring was continued for 2 hours to give a fine white precipitate. The solid was collected by filtration, and washed with water (4×10 mL). Drying under vacuum gave the desired peptide ester, Ac-Tyr(Bzl)-Leu-Pro-Arg(Pbf)-Pro-OMe (202 mg, 88%). The sample was used without further purification.

Next, Ac-Tyr(Bzl)-Leu-Pro-Arg(Pbf)-Pro-OMe (172 mg, 0.16 mmol) was dissolved in THF (5 mL) and water (5 mL) added dropwise with stifling. Lithium hydroxide monohydrate (12 eq) was added and stifling continued for 4 hours. The THF was removed using a rotary evaporator and the remaining aqueous solution diluted to 10 mL with water. 2N Hydrochloric acid (24 eq) was added dropwise. The resulting white solid was then collected by filtration, and washed with water (3×10 mL). Drying under vacuum gave the desired carboxylic acid, Ac-Tyr(Bzl)-Leu-Pro-Arg(Pbf)-Pro-OH (153 mg, 90%). The sample was used without further purification.

Next, Ac-Tyr(Bzl)-Leu-Pro-Arg(Pbf)-Pro-OH (35.3 mg, 0.033 mmol) was dissolved in DMF (0.5 mL). Butylamine (1 eq) and BOP (1 eq) were then added with stirring. DIEA (6 eq) was added, and the reaction mixture further stirred for 42 hours. The resultant mixture was quenched by addition to 0.1N potassium hydroxide (10 mL). Stirring was continued for 2 hours to give a fine white precipitate. The solid was collected by filtration and washed with water (5×2 mL). Drying under vacuum gave the desired butyl amine, Ac-Tyr(Bzl)-Leu-Pro-Arg(Pbf)-Pro-NHBu (35.2 mg, 94%). The sample was used without further purification.

Next, Ac-Tyr(Bzl)-Leu-Pro-Arg(Pbf)-Pro-NHBu (22.4 mg, 0.02 mmol) was dissolved in methanol (2 mL). 5% palladium on charcoal (19 mg, 25 mol %) was then added. The atmosphere was replaced by a hydrogen atmosphere at standard pressure and the reaction mixture stirred for 5 hours. The reaction mixture was filtered through a plug of silica/alumina catalyst support and evaporated to give a colorless oil. The oil was dissolved in TFA (2 mL) and stirred for 4 hours. Evaporation to dryness and purification by semi-prep HPLC gave the desired fully protected pentapeptide amine as a white solid, Ac-Tyr-Leu-Pro-Arg-Pro-NHBu.TFA (10.7 mg, 63%): ¹H NMR (deuteromethanol) δ=8.17 (d, 1H), 7.99 (d, 1H), 7.02 (m, 2H), 6.66 (m, 2H), 4.66 (m, 2H), 4.54 (dd, 1H), 4.36 (m, 2H), 3.79 (m, 1H), 3.70 (m, 1H), 3.60 (m, 1H), 3.56 (m, 1H), 3.19 (m, 6H), 2.98 (dd, 1H), 2.75 (dd, 1H), 2.19 (m, 2H), 2.04 (m, 2H), 1.94 (m, 2H), 1.92 (m, 1H), 1.90 (s, 3H), 1.88 (m, 2H), 1.70 (m, 4H), 1.54 (t, 2H), 1.50 (m, 2H), 1.37 (m, 2H), 0.94 (m, 9H) ppm; m/z=742.4 (M+H).

### Example 16

### Structure 16

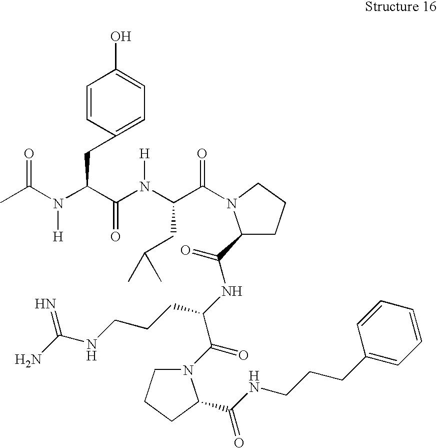

This Structure was created using a step-by-step synthesis process. First, Ac-Tyr(Bzl)-Leu-Pro-Arg(Pbf)-Pro-OH (35.5 mg, 0.033 mmol) was dissolved in DMF (0.5 mL). 3-phenylpropylamine (1 eq) and BOP (1 eq) were then added with stirring. DIEA (6 eq) was also added, and the reaction mixture stirred for 42 hours. The resultant mixture was quenched by addition to 0.1N potassium hydroxide (10 mL). Stirring was continued for 2 hours to give a fine white precipitate. The solid was collected by filtration, and washed with water (5×2 mL). Drying under vacuum gave the desired amine, Ac-Tyr(Bzl)-Leu-Pro-Arg(Pbf)-Pro-NH(CH₂)₃Ph (36.2 mg, 92%). The sample was used without further purification.

Next, Ac-Tyr(Bzl)-Leu-Pro-Arg(Pbf)-Pro-NH(CH₂)₃Ph (23.6 mg, 0.02 mmol) was dissolved in methanol (2 mL). 5% palladium on charcoal (19 mg, 25 mol %) was then added. The atmosphere was then replaced by a hydrogen atmosphere at standard pressure and the reaction mixture stirred for 5 hours. The reaction mixture was filtered through a plug of silica/alumina catalyst support and evaporated to give a colorless oil. The oil was then dissolved in TFA (2 mL) and stirred for 4 hours. Evaporation to dryness and purification by semi-prep HPLC gave the desired fully protected pentapeptide amine, Ac-Tyr-Leu-Pro-Arg-Pro-NH(CH₂)₃Ph.TFA, as a white solid (10.5 mg, 56%): ¹H NMR (deuteromethanol) δ=8.20 (d, 1H), 8.15 (t, 1H), 8.10 (d, 1H), 7.24 (m, 2H), 7.18 (m, 3H), 7.03 (d, 2H), 6.67 (d, 2H), 5.50 (s, 1H), 4.66 (m, 2H), 4.55 (m, 1H), 4.39 (m, 2H), 3.20 (m, 4H), 3.00 (m, 1H), 2.75 (m, 1H), 2.65 (m, 2H), 2.29 (m, 2H), 2.05 (m, 2H), 1.95 (m, 2H), 1.90 (s, 3H), 1.90 (m, 2H), 1.88 (m, 2H), 1.68, (m, 3H), 1.55 (m, 2H), 0.94 (m, 6H) ppm; m/z=804.4 (M+H).

### Example 17

### Structure 17

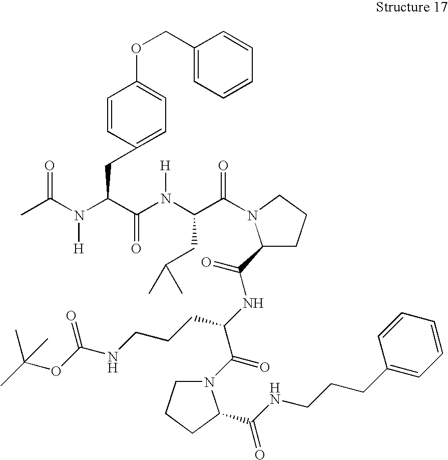

This Structure was created using a step-by-step synthesis process. First, Boc-Pro-OH (537.5 mg, 2.5 mmol) was dissolved in DMF (10 mL). 3-phenylpropylamine (1 eq) and BOP (1 eq) were added with stirring. DIEA (6 eq) was then added, and the reaction mixture stirred for 44 hours. The resultant mixture was then quenched by addition to 0.1N potassium hydroxide (180 mL). Stirring was continued for 2 hours to give an oily precipitate. The reaction mixture was extracted with ethyl acetate (3×75 mL) and the organic extracts washed with water (5×75 mL), brine (50 mL) and dried over sodium sulfate. Evaporation of the solvent gave a pale yellow oil, Boc-Pro-NH(CH₂)₃Ph (820 mg, 99%), which was used without further purification.

Next, Boc-Pro-NH(CH₂)₃Ph (166 mg, 0.5 mmol) was dissolved in CH₂Cl₂ (3 mL) and TFA (3 mL) added dropwise. After 45 minutes, the volatile components were removed using a rotary evaporator and the residual oil placed under vacuum for 2 hours. The resulting glass was dissolved in DMF (2.5 mL). Cbz-Orn(Boc)-OH (1 eq) and BOP (1 eq) were then added with stirring. DIEA (6 eq) was added, and the reaction mixture stirred for 70 hours. The resultant mixture was quenched by addition to 0.1N potassium hydroxide (40 mL). Stirring was continued for 1 hour to give an oily precipitate. The reaction mixture was extracted with ethyl acetate (2×50 mL) and the organic extracts washed with water (4×100 mL), brine (50 mL) and dried over sodium sulfate. Evaporation of the solvent gave a brown foam, Cbz-Orn(Boc)-Pro-NH(CH₂)₃Ph (262 mg, 90%), which was used without further purification.

Next, Cbz-Orn(Boc)-Pro-NH(CH₂)₃Ph (58 mg, 0.1 mmol) was dissolved in methanol (2 mL). 5% palladium on charcoal (12 mg, 25 mol %), and TFA (1 eq) were then added. The atmosphere was then replaced by a hydrogen atmosphere at standard pressure and the reaction mixture stirred for 4 hours. The reaction mixture was filtered through a plug of silica/alumina catalyst support and evaporated to give a colorless glass. The glass was dissolved in DMF (1 mL). Ac-Tyr(Bzl)-Leu-Pro-OH (1 eq) and BOP (1 eq) were then added with stifling. DIEA (6 eq) was added, and the reaction mixture further stirred for 24 hours. The resultant mixture was quenched by addition to 0.1N potassium hydroxide (15 mL). Stirring was continued for 2 additional hours to give a pale cream precipitate. The solid was collected by filtration, and washed with water (10×2 mL). Drying under vacuum gave the desired amine, Ac-Tyr(Bzl)-Leu-Pro-Orn(Boc)-Pro-NH(CH₂)₃Ph (96 mg, 100%). The reaction product was purified by semi preparative reverse phase HPLC to give a white solid: ¹H NMR (DMSO d₆) δ=8.10 (d, 8.4 Hz, 1H), 7.95 (m, 2H), 7.77 (t, J=5.1 Hz, 1H), 7.43-7.15 (m, 10H), 7.12 (d, J=9.1 Hz, 2H), 6.87 (d, J=8.4 Hz, 2H), 6.73 (m, 1H), 5.03 (s, 2H), 4.54-4.37 (m, 3H), 4.34 (dd, J=4.8, 8.4 Hz, 1H), 4.20 (dd, J=4.5, 8.4, 1H), 3.61-3.40 (m, 4H), 3.10-2.85 (m, 5H), 2.60 (dd, J=10.4, 14.4 Hz, 1H), 2.54 (m, 2H), 2.06-1.75 (m, 8H), 1.73, (s, 3H), 1.70-1.57 (m, 4H), 1.43 (m, 4H), 1.35 (s, 9H), 1.23 (m, 1H), 0.87 (m, 6H) ppm; m/z=952.5 (M+H).

### Example 18

### Structure 18

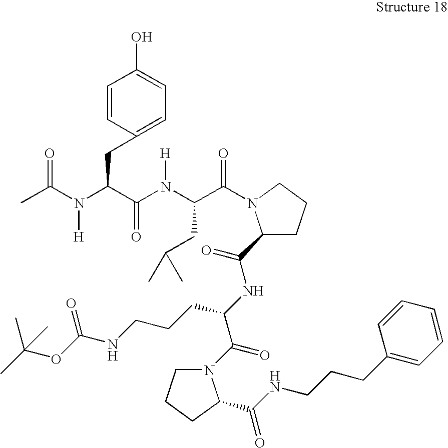

First, Ac-Tyr(Bzl)-Leu-Pro-Orn(Boc)-Pro-NH(CH₂)₃Ph (9.5 mg, 0.01 mmol) was dissolved in methanol (1 mL). 5% palladium on charcoal (2.4 mg, 10 mol %) was next added. The atmosphere was then replaced by a hydrogen atmosphere at standard pressure and the reaction mixture stirred for 24 hours. The reaction mixture was filtered through a plug of silica/alumina catalyst support and evaporated to give a colorless glass. The crude product was purified by semi preparative reverse phase HPLC to give a white solid, Ac-Tyr-Leu-Pro-Orn(Boc)-Pro-NH(CH₂)₃Ph (3.9 mg, 45%): ¹H NMR (DMSO d₆) δ=9.14 (s, 1H), 8.05 (d, J=8.6 Hz, 1H), 7.95 (d, J=7.5 Hz, 1H), 7.91 (d, J=8.6 Hz, 1H), 7.77 (t, J=8.7 Hz, 1H), 7.26 (m, 2H), 7.16 (m, 3H), 6.98 (d, J=8.6 Hz, 2H), 6.73 (d, J=7.8 Hz, 2H), 6.60 (d, J=8.6 Hz, 1H), 4.50 (m, 1H), 4.40 (m, 2H), 4.33 (dd, J=4.4, 8.6 Hz, 1H), 4.20 (dd, J=4.5, 7.5 Hz, 1H), 3.54 (m, 2H), 3.46 (m, 2H), 3.12-2.78 (m, 5H), 2.53 (m, 3H), 2.09-1.75 (m, 9H), 1.73 (s, 3H), 1.65 (m, 4H), 1.42 (m, 4H), 1.35 (s, 9H), 0.87 (m, 6H) ppm; m/z=862.4 (M+H).

### Example 19

### Structure 19

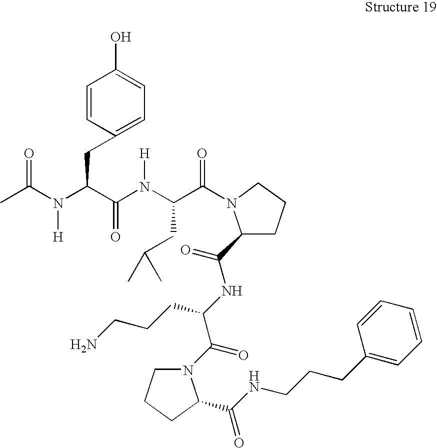

First, Ac-Tyr-Leu-Pro-Orn(Boc)-Pro-NH(CH₂)₃Ph (16.5 mg, 0.02 mmol) was dissolved in CH₂Cl₂ (0.5 mL) and TFA (0.5 mL) added dropwise. After 60 minutes, the volatile components were removed using a rotary evaporator and the residual oil placed under vacuum for 16 hours. The crude product was purified by semi preparative reverse phase HPLC to give a white solid, Ac-Tyr-Leu-Pro-Orn-Pro-NH(CH₂)₃Ph (13.1 mg, 75%): ¹H NMR (DMSO d₆) δ=9.14 (br s, 1H), 8.09 (d, J=9.0 Hz, 1H), 8.00 (d, J=6.7 Hz, 1H), 7.94 (d, J=8.0 Hz, 1H), 7.84 (t, J=5.6 Hz, 1H), 7.61 (br s, 3H), 7.25 (m, 2H), 7.15 (m, 3H), 6.97 (d, J=7.9 Hz, 2H), 6.59 (d, J=8.8 Hz, 2H), 4.48 (m, 2H), 4.40 (m, 1H), 4.29 (dd, J=4.6, 8.8 Hz, 1H), 4.19 (dd, J=4.4, 7.9 Hz, 1H), 3.54 (m, 4H), 3.00 (m, 2H), 2.75 (m, 3H), 2.53 (m, 3H), 2.01 (m, 2H), 1.82 (m, 6H), 1.72 (s, 3H), 1.62 (m, 7H), 1.39 (m, 2H), 0.86 (m, 6H) ppm; m/z=762.4 (M+H).

### Example 20

### Structure 20

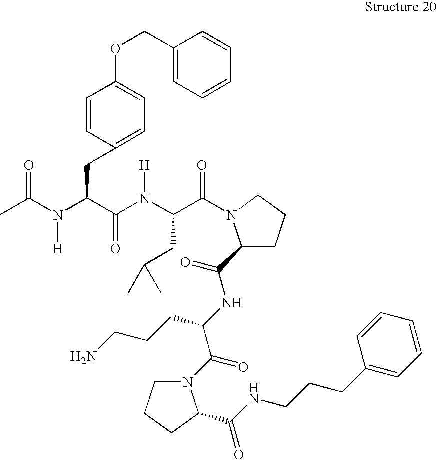

First, Ac-Tyr(Bzl)-Leu-Pro-Orn(Boc)-Pro-NH(CH₂)₃Ph (9.5 mg, 0.01 mmol) was dissolved in CH₂Cl₂ (0.5 mL) and TFA (0.5 mL) added dropwise. After 60 minutes, the volatile components were removed using a rotary evaporator and the residual oil placed under vacuum for 16 hours. The crude product was purified by semi preparative reverse phase HPLC to give a white solid, Ac-Tyr(Bzl)-Leu-Pro-Orn-Pro-NH(CH₂)₃Ph (6.2 mg, 64%): ¹H NMR (DMSO d₆) δ=8.11 (d, J=8.0 Hz, 1H), 8.05 (d, J=7.2 Hz, 1H), 7.99 (d, J=8.1 Hz, 1H), 7.86 (t, J=6.0 Hz, 1H), 7.62 (br m, 3H), 7.45-7.23 (m, 7H), 7.20-7.10 (m, 5H), 6.88 (m, 2H), 5.04 (s, 2H), 4.49 (m, 2H), 4.31 (dd, J=4.0, 8.1 Hz, 1H), 4.22 (dd, J=4.1, 8.1 Hz, 1H), 3.58 (m, 1H), 3.54 (m, 1H), 3.03 (m, 2H), 2.83 (dd, J=4.1, 14.4 Hz, 1H) 2.77 (m, 2H), 2.60 (dd, J=10.0, 14.4 Hz, 1H), 2.54 (m, 2H), 2.03 (m, 2H), 1.86 (m, 4H), 1.74 (s, 3H), 1.66 (m, 5H), 1.42 (m, 2H) 0.88 (m, 6H) ppm; m/z=852 (M+H).

### Example 21

### Structure 21

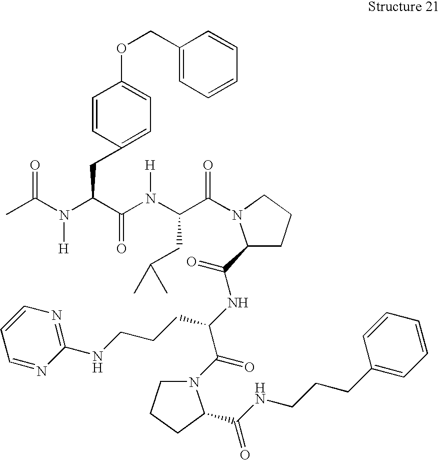

This Structure was created using a step-by-step synthesis process. First, Boc-Pro-NH(CH₂)₃Ph (802 mg, 2.415 mmol) was dissolved in CH₂Cl₂ (5 mL) and TFA (5 mL) added dropwise. After 45 minutes, the volatile components were removed using a rotary evaporator and the residual oil placed under vacuum for 2 hours. The resulting glass was dissolved in DMF (10 mL). Boc-Orn(Cbz)-OH (1 eq) and BOP (1 eq) were then added with stifling. DIEA (6 eq) was also added, and the reaction mixture stirred for 17 hours. The resultant mixture was quenched by addition to 0.1N potassium hydroxide (150 mL). Stirring was continued for 3 hours to give a milky white emulsion. The reaction mixture was extracted with ethyl acetate (2×60 mL) and the organic extracts washed with water (4×100 mL), brine (50 mL) and dried over sodium sulfate. Evaporation of the solvent gave a sticky golden foam, Boc-Orn(Cbz)-Pro-NH(CH₂)₃Ph (1081 mg, 77%), which was used without further purification.

Next, Boc-Orn(Cbz)-Pro-NH(CH₂)₃Ph (655 mg, 1.13 mmol) was dissolved in methanol (20 mL). 5% palladium on charcoal (136 mg, 5 mol %), and TFA (1 eq) were then added. Next, the atmosphere was replaced by a hydrogen atmosphere at standard pressure and the reaction mixture stirred for 3 hours. The reaction mixture was filtered through a plug of silica/alumina catalyst support and evaporated to give a white foam/glass. The crude product was purified by semi preparative reverse phase HPLC to give a white solid, Boc-Orn-Pro-NH(CH₂)₃Ph (631 mg, 100%), which was used without further purification.

Next, Boc-Orn-Pro-NH(CH₂)₃Ph (33 mg, 0.06 mmol) was dissolved in DMSO (250 L). 2-Chloropyrimidine (2 eq) and potassium hydroxide (2.2 eq) were added and the reaction mixture heated to 65° C. for 20 hours. The crude reaction mixture was purified by reverse phase semi preparative HPLC to give the desired product, Boc-Orn(2-pyrimidine)-Pro-NH(CH₂)₃Ph (6.8 mg, 34%): ¹H NMR (DMSO d₆) δ=8.48 (d, 2H), 7.23 (m, 2H), 7.18 (m, 2H), 7.14 (m, 1H), 6.87 (t, 1H), 4.39 (m, 2H), 3.83 (m, 1H), 3.65 (m, 1H), 3.47 (t, 2H), 3.19 (t, 2H), 2.62 (t, 2H), 2.22 (m, 1H), 2.07 (m, 1H), 1.97 (m, 1H), 1.90, (m, 1H), 1.86 (m, 1H), 1.81 (m, 6H), 1.72 (m, 1H), 1.42 (s, 9H) ppm.

Next, Boc-Orn(2-pyrimidine)-Pro-NH(CH₂)₃Ph (6 mg, 0.01 mmol) was dissolved in CH₂Cl₂ (0.5 mL) and TFA (0.5 mL) added dropwise. After 45 minutes, the volatile components were removed using a rotary evaporator and the residual oil placed under vacuum for 2 hours. The glass was dissolved in DMF (0.2 mL), and Ac-Tyr(Bzl)-Leu-Pro-OH (1 eq) and BOP (1 eq) were added with stirring. DIEA (6 eq) was also added, and the reaction mixture stirred for 120 hours. The resultant mixture was quenched by addition to 0.1N potassium hydroxide (4 mL). Stirring was continued for 2 hours to give a pale tan precipitate. The solid was collected by filtration, and washed with water (4×2 mL). Drying under vacuum gave the desired amine, Ac-Tyr(Bzl)-Leu-Pro-Orn(2-pyrimidine)-Pro-NH(CH₂)₃Ph (4.8 mg, 50%). The reaction product was purified by semi preparative reverse phase HPLC to give a white solid: ¹H NMR (DMSO d₆) δ=8.23 (m, 2H), 8.18 (m, 1H), 7.99 (d, J=9.2 Hz, 1H), 7.96 (d, J=9.6 Hz, 1H), 7.76 (t, J=6.0 Hz, 1H), 7.44-7.08 (m, 11H), 6.90 (d, J=9.6 Hz, 2H), 6.51 (m, 3H), 5.03 (s, 2H), 4.51 (m, 1H), 4.46 (m, 1H), 4.44 (m, 1H), 4.35 (m, 1H), 4.20 (m, 1H), 3.54 (m, 4H), 3.23 (m, 2H), 3.09-2.83 (m, 5H), 2.60 (m, 1H), 2.06-1.73 (m, 8H), 1.73 (s, 3H), 1.69-1.48 (m, 7H), 1.42 (m, 2H), 0.86 (m, 6H) ppm; m/z=930.5.

### Example 22

### Structure 22

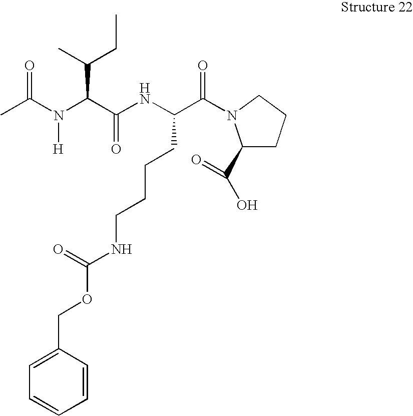

Boc-Lys(Cbz)-Pro-OMe: Proline methyl ester hydrochloride (166 mg, 1 mmol) and Boc-Lys(Cbz)-OH (1 mmol, 380 mg) dissolved in DMF (10 mL) and BOP (1 mmol, 442 mg) added with stirring. DIEA (7 mmol, 1.22 mL) was added dropwise and mixture stirred for 7 d. Reaction mixture quenched by addition to water (150 mL) and extracted with ethyl acetate (2×100 mL). Combined organic phases washed with water (4×100 mL), and brine (100 mL), then dried over sodium sulfate. Evaporation of solvent gave a colorless oil (445 mg, 90%) which was used without further purification.

Boc-Ile-Lys(Cbz)-Pro-OMe: Boc-Lys(Cbz)-Pro-OMe (123 mg, 0.25 mmol) was dissolved in CH₂Cl₂ (1.25 mL) and TFA (1.25 mL) added dropwise. After 45 min the volatile components were removed on a rotary evaporator and the residual oil place under vacuum for 2 h. The resulting glass was dissolved in DMF (2.5 mL) and Boc-Ile-OH.½H₂O (1 eq, 60 mg), BOP (1 eq, 111 mg) added with stirring. DIEA (6 eq, 0.261 mL) was added dropwise and mixture stirred for 44 h. The reaction mixture was quenched by addition to water (75 mL) producing a gummy precipitate which was recovered by extraction with ethyl acetate (30 mL). The organic extract was washed with water (3×25 mL) and brine (20 mL), before drying over sodium sulfate. Evaporation of the solvent gave Boc-Ile-Lys(Cbz)-Pro-OMe as a white foam (125 mg, 83%): predominantly one peak by analytical hplc, used without further purification.

Ac-Ile-Lys(Cbz)-Pro-OMe: Boc-Ile-Lys(Cbz)-Pro-OMe (125 mg, 0.207 mmol) was dissolved in CH₂Cl₂ (2 mL) and TFA (2 mL) added dropwise. After 45 min the volatile components were removed on a rotary evaporator and the residual oil place under vacuum for 18 h. The resulting glass was dissolved in pyridine (1 mL) and acetic anhydride (4 eq) added with stirring and mixture stirred at 0° C. for 5 h. After 5 h the reaction mixture was allowed to warm to r.t over 30 min. The reaction mixture was quenched by addition to 2N hydrochloric acid (50 mL) and extracted with ethyl acetate (30 mL). The organic phase was washed with 2N hydrochloric acid (2×50 mL) and brine (25 mL), then dried over sodium sulfate. Evaporation of solvent gave a yellowish foam/solid (95 mg, 84%): predominantly one peak by analytical hplc, used without further purification.

Ac-Ile-Lys(Cbz)-Pro-OH: Ac-Tyr(Bzl)-Leu-Pro-OMe (95 mg, 0.174 mmol) was dissolved in THF (4 mL) and water (4 mL) added dropwise with stifling. Lithium hydroxide monohydrate (12 eq) was added and stirring continued for 4 h. The THF was removed on a rotary evaporator and the remaining aqueous solution diluted to 14 mL with water. 2N Hydrochloric acid (24 eq) was added dropwise and the resulting yellow oily solid was extracted with ethyl acetate (10 mL) The organic extract was dried over sodium sulfate and evaporated to give the desired carboxylic as a yellow foam (81 mg, 88%): ¹H NMR (DMSO d₆) δ=12.4 (br s, 1H), 7.97 (d, J=9.2 Hz, 1H), 7.81 (d, J=7.5 Hz, 1H), 7.36-7.24 (m, 5H), 7.19 (t, J=5.0 Hz, 1H), 4.96 (s, 2H), 4.38 (m, 2H), 4.16 (m, 2H), 3.68 (m, 1H), 3.48 (m, 1H), 2.92 (m, 2H), 2.09 (m, 1H), 1.83 (m, 2H), 1.80 (s, 3H), 1.60 (m, 2H), 1.47 (m, 1H), 1.39-1.17 (m, 5H), 1.02 (m, 1H), 0.83 (m, 1H), 0.75 (m, 6H) ppm; m/z=533 (M+H).

### Example 23

### Structure 23

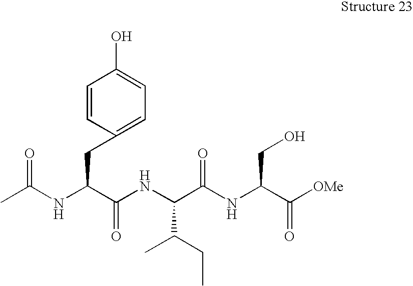

Boc-Ile-Ser(Bzl)-OMe: To a solution of Boc-Ile-OH.0.5H₂O (300 mg, 1.25 mmol) and H-Ser(Bzl)-OMe.HCl (1 eq) in DMF (10 mL) was added BOP (1 eq) followed by DIEA (6 eq) and the mixture was allowed to stand for 17 h. The reaction mixture was added dropwise into 150 mL H₂O and ˜5 mL brine was added. After stirring for 2 h, a white solid was collected by filtration, washed with 0.1 N HCl(aq) (2×10 mL), 0.1 N KOH(aq) (2×10 mL), and H₂O (10 mL). Drying under high vacuum afforded Boc-Ile-Ser(Bzl)-OMe (336 mg, 64%) as a white solid (single peak by analytical HPLC), which was carried on without further purification.

Boc-Tyr(Bzl)-Ile-Ser(Bzl)-OMe: Boc-Ile-Ser(Bzl)-OMe (150 mg, 0.355 mmol) was dissolved in CH₂Cl₂ (2 mL), TFA (2 mL) was added, and the solution was allowed to stand for 45 min. The CH₂Cl₂ and TFA were evaporated under reduced pressure, residual TFA was removed by evaporation with toluene, and the resulting residue (H-Ile-Ser(Bzl)-OMe.TFA) was dried under high vacuum overnight. To a solution of H-Ile-Ser(Bzl)-OMe.TFA and Boc-Tyr(Bzl)-OH (1 eq) in DMF (4 mL) was added BOP (1 eq) followed by DIEA (6 eq) and the reaction mixture was allowed to stand for 17 h. The reaction mixture was added dropwise into 75 mL H₂O and after stifling for 1 h, a white solid was collected by filtration. Drying under high vacuum afforded Boc-Tyr(Bzl)-Ile-Ser(Bzl)-OMe (228 mg, 95%) as a white solid, which was carried on without further purification.

Ac-Tyr(Bzl)-Ile-Ser(Bzl)-OMe: Boc-Tyr(Bzl)-Ile-Ser(Bzl)-OMe (228 mg, 0.337 mmol) was dissolved in CH₂Cl₂ (2 mL), TFA (2 mL) was added, and the solution was allowed to stand for 45 min. The CH₂Cl₂ and TFA were evaporated under reduced pressure, residual TFA was removed by evaporation with toluene, and the resulting residue (H-Tyr(Bzl)-Ile-Ser(Bzl)-OMe.TFA) was dried under high vacuum overnight. The residue was dissolved in pyridine (8 mL) and the resulting solution was cooled to 5° C. Acetic anhydride (6 eq) was added and the reaction mixture was stirred at 5° C. for 4 h. The mixture was poured into EtOAc (100 mL) and washed with 0.1 N HCl(aq) (2×40 mL), sat. NaHCO₃(aq) (2×40 mL), brine (40 mL), and H₂O (40 mL) and dried over Na₂SO₄. Evaporation of the solvent gave a crystalline solid. The solid was suspended in 5 mL of hot EtOAc, the mixture was allowed to cool, the EtOAc was removed with a pipette, and the solid was rinsed with hexanes (2×5 mL). Drying under high vacuum afforded Ac-Tyr(Bzl)-Ile-Ser(Bzl)-OMe (183 mg, 88%) as a white solid (single peak by analytical HPLC), which was used without further purification.

Ac-Tyr-Ile-Ser-OMe: To a solution of Ac-Tyr(Bzl)-Ile-Ser(Bzl)-OMe (183 mg, 0.297 mmol) in MeOH (5 mL) was added 5 wt % Pd/C (0.2 eq Pd) followed by TFA (5 eq). The vessel was flushed with H₂(g) and the reaction mixture was stirred under H₂(g) atmosphere for 2 d. The mixture was filtered through packed cotton to removed Pd/C and the filtrate was evaporated under reduced pressure leaving a white solid. Drying under high vacuum afforded Ac-Tyr-Ile-Ser-OMe (125 mg, 96%) as a white solid: ¹H NMR (400 MHz, DMSO-d₆): δ=9.14 (s, 1H), 8.26 (d, J=7.4 Hz, 1H), 8.00 (d, J=8.4 Hz, 1H), 7.81 (d, J=8.9 Hz, 1H), 7.00 (d, J=9.0 Hz, 2H), 6.61 (d, J=9.0 Hz, 2H), 5.03 (t, J=5.7 Hz, 1H), 4.48-4.41 (m, 1H), 4.35-4.25 (m, 2H), 3.73-3.67 (m, 1H), 3.65-3.58 (m, 1H), 3.60 (s, 3H), 2.84 (dd, J=14.0, 4.0 Hz, 1H), 2.58 (dd, J=14.0, 10.1 Hz, 1H), 1.74 (s, 3H), 1.76-1.66 (m, 1H), 1.48-1.37 (m, 1H), 1.12-0.99 (m, 1H), 0.86-0.78 (m, 6H); ESI-MS: m/z calcd for C₂₁H₃₂N₃O₇ (M+H⁺) 438.2. found 438.2; single peak by analytical HPLC.

### Example 24

### Structure 24

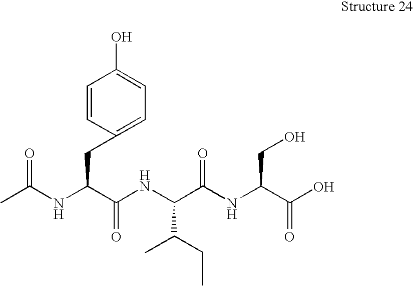

Ac-Tyr-Ile-Ser-OH: Ac-Tyr-Ile-Ser-OMe (90 mg, 0.206 mmol) was dissolved in THF (8 mL) and H₂O (7 mL) was added dropwise with stirring. LiOH.H₂O (12 eq) was added and the reaction mixture was stirred for 4 h. The THF was removed under reduced pressure, the remaining aqueous mixture was diluted to 10 mL with H₂O, acidified by addition of 2 N HCl(aq) (24 eq) and washed with CH₂Cl₂ (3×20 mL). The aqueous layer was co-evaporated with toluene under reduced pressure leaving a solid. 25 mg of the crude mixture was purified by preparative HPLC affording Ac-Tyr-Ile-Ser-OH (3 mg) as a white solid: ¹H NMR (400 MHz, DMSO-d₆): δ=9.13 (s, 1H), 8.08 (d, J=7.3 Hz, 0.5H), 8.00 (d, J=8.6 Hz, 1H), 8.00-7.95 (m, 0.5H), 7.82 (d, J=8.9 Hz, 1H), 7.01 (d, J=8.6 Hz, 2H), 6.61 (d, J=8.6 Hz, 2H), 4.95 (bs, 1H), 4.48-4.41 (m, 1H), 4.30-4.21 (m, 2H), 3.73-3.65 (m, 1H), 3.65-3.58 (m, 1H), 2.85 (dd, J=14.0, 4.1 Hz, 1H), 2.58 (dd, J=14.0, 10.4 Hz, 1H), 1.74 (s, 3H), 1.76-1.67 (m, 1H), 1.50-1.38 (m, 1H), 1.12-0.99 (m, 1H), 0.88-0.77 (m, 6H). ESI-MS: m/z calcd for C₂₀H₃₀N₃O₇ (M+H⁺) 424.2. found 424.2; single peak by analytical HPLC.

### Example 25

### Structure 25- and

### Example 26

### Structure 26

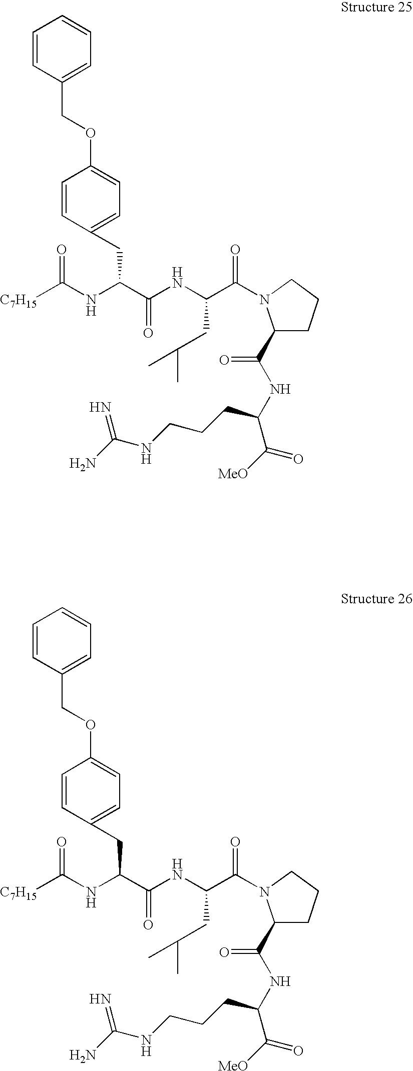

Octanoyl-Tyr(Bzl)-Leu-Pro-OMe: Boc-Tyr(Bzl)-Leu-Pro-OMe (119 mg, 0.2 mmol) was dissolved in CH₂Cl₂ (2 mL) and TFA (2 mL) added dropwise. After 45 min the volatile components were removed on a rotary evaporator and the residual oil placed under vacuum for 2 h. The resulting glass was dissolved in dimethylformamide (2 mL) and octanoic acid (1 eq, 28.8 mg, 31.6 μL) added. BOP (1.0 eq., 88.4 mg) and then diisopropylethylamine (DIEA) (6 eq., 155 mg, 209 μL) was added with stirring and the mixture stirred for 90 h. The reaction mixture was quenched by addition to water (35 mL) and stirred for 1 h. Sodium chloride (1 gm) was added and stirring continued for 4 h. The resultant tan precipitate was collected by filtration and washed with water (4×5 mL). The remaining solid was dried under vacuum to give a tan solid (116 mg, 93%). The sample was used without further purification.

Octanoyl-Tyr(Bzl)-Leu-Pro-OH: Octanoyl-Tyr(Bzl)-Leu-Pro-OMe (62.1 mg, 0.1 mmol) was dissolved in THF (2 mL) and water (1.75 mL) added dropwise with stifling. Lithium hydroxide monohydrate (12 eq, 50.4 mg) was added and stirring continued for 4 h. The THF was removed on a rotary evaporator and the remaining aqueous solution diluted to 10 mL with water. 2N Hydrochloric acid (24 eq, 1.2 mL)) was added dropwise and stifling was continued for 1 h. The resulting tan solid was collected by filtration, and washed with water (5×5 mL). Drying under vacuum gave the desired carboxylic acid (57 mg, 94%). The sample was used without further purification.

Octanoyl-Tyr(Bzl)-Leu-Pro-Arg(Pbf)-OMe: Octanoyl-Tyr(Bzl)-Leu-Pro-OH (30.4 mg, 0.05 mmol) was dissolved in DMF (0.5 mL) and H-Arg(Pbf)-OMe hydrochloride (1 eq, 24 mg) and BOP (1 eq. 22.1 mg) were added with stirring. DIEA (7 eq., 45 mg, 61 μL) was added, and the reaction mixture stirred for 65 h. The resultant mixture was quenched by addition to water (10 mL) and stirred for 1 h. Sodium chloride (˜0.5 g) added and stifling continued for 2 h to give a fine tan precipitate. The solid was collected by filtration, and washed with water (5×2 mL). Drying under vacuum gave the desired ester (50 mg, 96%). The sample was used without further purification.

Octanoyl-Tyr(Bzl)-Leu-Pro-Arg-OMe trifluoroacetate (Example 26) and Octanoyl-(D)-Tyr(Bzl)-Leu-Pro-Arg-OMe trifluoroacetate (Example 25): Octanoyl-Tyr(Bzl)-Leu-Pro-Arg(Pbf)-OMe (50 mg, 0.048 mmol) was dissolved in TFA (1 mL) and triiopropylsilane (xs, 3 drops) added. The reaction mixture was stirred for 2 h, evaporated to dryness and purified by semi-prep HPLC to give the desired fully deprotected tetrapeptide ester trifluoroacetate salt Example 26 as a white solid (25 mg, 58%): ¹H NMR (d6-DMSO) δ=8.14 (d, 1H), 7.94 (d, 1H), 7.88 (d, 1H), 7.43 (m, 1H), 7.32-7.25 (m, 5H), 7.10 (d, 2H), 6.85 (d, 2H), 5.01 (s, 2H), 4.50 (m, 1H), 4.46 (m, 1H), 4.32 (m, 1H), 4.18 (m, 1H), 3.58 (s, 3H), 3.44 (m, 2H), 3.07 (m, 2H), 2.86 (m, 1H), 2.61 (m, 1H), 2.01 (m, 1H), 1.97 (t, 2H), 1.92-1.65 (m, 3H), 1.57 (m, 1H), 1.50 (m, 2H), 1.39 (m, 2H), 1.31 (m, 2H), 1.20 (m, 2H), 1.14 (m, 4H), 1.05 (m, 2H), 0.88 (d, 3H), 0.86 (d, 3H), 0.85 (m, 1H), 0.80 (t, 3H) ppm; m/z=778.5 (M+H); and the (D)-Tyr epimer salt, Example 25, (5.5 mg, 13%): ¹H NMR (d6-DMSO)=8.22 (d, 1H), 7.90 (br d, 1H), 7.78 (d, 1H), 7.43 (br t, 1H), 7.20-7.07 (m, 5H), 6.92 (s, 2H), 6.81 (m, 2H), 6.62 (d, 2H), 4.49 (m, 1H), 4.41 (m, 1H), 4.31 (m, 1H), 4.07 (m, 1H), 3.79 (d, 1H), 3.74 (d, 1H), 3.58 (s, 3H), 3.54 (m, 2H), 3.42 (m, 2H), 3.08 (m, 2H), 2.78 (dd, 1H), 2.53 (dd, 1H), 2.04-1.64 (m, 6H), 1.58 (m, 1H), 1.50 (m, 2H), 1.38 (m, 2H), 1.34 (m, 2H), 1.26-1.04 (m, 8H), 0.86 (d, 3H), 0.84 (d, 3H), 0.81 (t, 3H) ppm; m/z=778.5 (M+H)

### Example 27

### Structure 27 and

### Example 28

### Structure 28

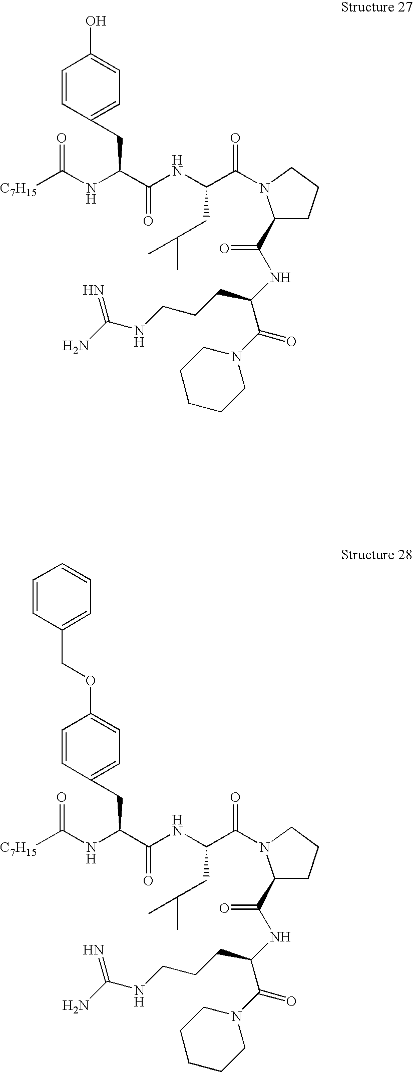

Octanoyl-Tyr(Bzl)-Leu-Pro-Arg(Pbf)-OH: Octanoyl-Tyr(Bzl)-Leu-Pro-Arg(Pbf)-OMe (130.1 mg, 0.125 mmol) was dissolved in THF (2.5 mL) and water (1.5 mL) added dropwise with stifling. Lithium hydroxide monohydrate (12 eq. 63 mg) was added and stirring continued for 4 h. The THF was removed on a rotary evaporator and the remaining aqueous solution diluted to 12 mL with water. 2N Hydrochloric acid (24 eq., 1.2 mL) was added dropwise and the reaction mixture stirred overnight. The resulting off white solid was collected by filtration, and washed with water (6×4 mL). Drying under vacuum gave the desired carboxylic acid (118 mg, 92%). The sample was used without further purification.

Octanoyl-Tyr(Bzl)-Leu-Pro-Arg(Pbf)-N(CH₂)₅: Octanoyl-Tyr(Bzl)-Leu-Pro-Arg(Pbf)-OH (51.4 mg, 0.05 mmol) was dissolved in DMF (0.5 mL) and piperidine (1 eq., 4.25 mg, 5.0 μL) and BOP (1 eq., 22.1 mg) were added with stifling. DIEA (7 eq., 45 mg, 61 μL) was added, and the reaction mixture stirred for 16 h. The resultant mixture was quenched by addition to water (15 mL). Stifling was continued for 3 h to give a fine tan precipitate. The solid was collected by filtration, and washed with water (5×5 mL). Drying under vacuum gave the desired piperidinyl amide (49 mg, 89%). The sample was used without further purification.

Octanoyl-Tyr-Leu-Pro-Arg-N(CH₂)₅ trifluoroacetate and Octanoyl-Tyr(Bzl)-Leu-Pro-Arg-N(CH₂)₅ trifluoroacetate: Octanoyl-Tyr(Bzl)-Leu-Pro-Arg(Pbf)-N(CH₂)₅ (36.4 mg, 0.033 mmol) was dissolved in TFA (1 mL) and triisopropylsilane (xs, 1 drop) added. The reaction mixture was stirred for 2 h, evaporated to dryness and purified by semi-prep HPLC gave the desired fully deprotected tetrapeptide amide trifluoroacetate salt, Example 3, as a white solid (5.1 mg, 18%): ¹H NMR (d6-DMSO)=8.07 (d, 1H), 7.92 (d, 1H), 7.84 (d, 1H), 7.37 (t, 1H), 6.96 (d, 2H), 6.58 (d, 2H), 4.64 (m, 1H), 4.49 (m, 1H), 4.41 (m, 1H), 4.30 (dd, 1H), 3.57 (m, 1H), 3.46 (m, 1H), 3.38 (m, 4H), 3.07 (m, 2H), 2.79 (dd, 1H), 2.56 (dd, 1H), 2.02 (m, 1H), 1.97 (t, 2H), 1.93-1.68 (m, 3H), 1.68-1.51 (m, 3H), 1.51-1.28 (m, 10H), 1.21 (m, 2H), 1.16 (m, 4H), 1.08 (m, 2H), 0.86 (m, 6H), 0.82 (t, 3H) ppm; m/z=741.5 (M+H); and the benzyl protected tyrosine analog, Example 4, as a white solid (8.9 mg, 28%): ¹H NMR (d6-DMSO) δ=8.09 (d, 1H), 7.96 (d, 1H), 7.88 (d, 1H), 7.40-7.26 (m, 6H), 7.10 (d, 2H), 6.84 (d, 2H), 5.01 (s, 2H), 4.64 (m, 1H), 4.50 (m, 1H), 4.43 (m, 1H), 4.31 (dd, 1H), 3.58 (m, 1H), 3.46 (m, 1H), 3.37 (m, 2H), 3.07 (m, 4H), 2.85 (dd, 1H), 2.61 (dd, 1H), 2.01 (m, 1H), 1.97 (t, 2H), 1.86 (m, 1H), 1.81 (m, 1H), 1.75 (m, 1H), 1.65-1.52 (m, 4H), 1.50-1.28 (m, 10H), 1.21 (m, 2H), 1.16 (m, 2H), 1.04 (m, 3H), 0.87 (d, 3H), 0.85 (d, 3H), 0.81 (t, 3H) ppm; m/z=831.5 (M+H).

### Example 29

### Structure 29

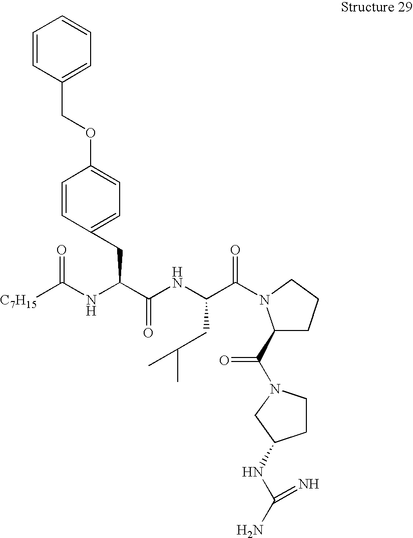

(S)-3-amino)-N-Fmoc-pyrrolidine: (S)-3-(Boc-amino)pyrrolidine (485 mg, 2.6 mmol) in 10 ml CH₂Cl₂ was added with stirring to a 20 ml aqueous solution of 10% NaHCO₃ at 0° C., followed by drop by drop addition of 10 ml CH₂Cl₂ solution of Fmoc-Cl (884 mg, 3.4 mmol) for 15 min. The reaction was completed with in an hour as determined from silica TLC plate. The organic layer was separated and dried to remove organic volatiles to obtained white crude solid. Diethyl ether (100 ml) was used to wash off any extra Fmoc-OH from the product. After drying, the crude white solid, (S)-3-(Boc-amino)-N-Fmoc-pyrrolidine (yield: 98%) was further treated with 20 ml of (1:1) CH₂Cl₂:TFA solution for an hour. The volatile component was removed by rotary evaporator and the crude solid was further dried under vacuum for 12 h to obtain 790 mg (98%) of (S)-3-amino) Fmoc-N Pyrrolidine.

(S)-3-(NCS)—N-Fmoc-pyrrolidine: (S)-3-amino)-N-Fmoc-pyrrolidine (790 mg, 2.56 mmol), was dissolved in 25 ml of anhydrous THF under argon at 0° C. To the stifling solution, CS₂ (1.4 ml, 23 mmol), DIEA (1.6 ml, 9.3 mmol), was added through syringes and the reaction was allowed for 90 min at cold temperature. Hydrogen peroxide (30% wt./vol.; 3 ml, 26.6 mmol), was added to the reaction product and stirring continued for an hour until the solution temperature reached room temperature. The reaction mixture was diluted with diethyl ether (100 ml) and quenched with 150 ml of NaH₂PO₄ (2.07 gm, 0.1M). The organic layer was removed by separating funnel followed by washing with water (100 ml) and brine solution (50 ml). Finally, the organic layer was dried over anhydrous Na₂SO₄. The resulting semi dried organic reaction mixture was dried first by rotary evaporator followed by high vacuum pump to obtained light yellow crude solid. This solid crude was subjected to column chromatography using silica. Fractions collected using 80:20 (vol/vol) Hexane:EtOAc mixture solvent system yielded pure white powder of (S)-3-(NCS)—N-Fmoc-pyrrolidine (306 mg, 35%).

Coupling reaction of (S)-3-(NCS)—N-Fmoc-pyrrolidine with Rink Amide AM resin: The detail preparation of Fmoc deprotected Rink Amide AM resin was described previously under Example 1 section. Coupling between these two reagents was achieved by reacting S)-3-(NCS)—N-Fmoc-pyrrolidine (105 mg, 0.298 mmol) and Rink Amide AM resin (112 mg @0.68 mmol/g) in 1.5 ml anhydrous THF solution in the presence of excess base DIEA (60 ul, 0.35 mmol) overnight. The resins were washed with 15 ml THF and dried under vacuum. Yield 133 mg. The resin was treated with 20% v/v piperidine in DMF (10 ml) for half an hour, followed by filtration and subsequent washing with DMF. The procedure was repeated twice to ensure complete de protection of Fmoc moiety from pyrrolidine unit. The resins was dried overnight under vacuum to obtain 120 mg of (S)-3-(NH—C(═S)—NH-AM Rink Amide) pyrrolidine resin. The resin was treated with amino acids coupling cocktail comprising an Fmoc protected aminoacids (5 eq.), PyBOP (5 eq.), HOBt (5 eq.), and DIEA (10 eq.) in 10 ml DMF and the reaction was carried out overnight. The resin was washed thoroughly with 15 ml DMF (5×3 ml) to complete one synthesis cycle followed by three separate Fmoc deprotection reactions with 20% piperidine in DMF except for the last cycle when octanoic acid was used as the coupling unit. The amino acids employed were consecutively, Fmoc-Pro-OH (117 mg, 0.35 mmol, overnight reaction), Fmoc-Leu-OH (110 mg, 0.35 mmol, overnight reaction), Fmoc-Tyr(Bzl)-OH (129 mg, 0.26 mmol, reaction time 72 h) and Octanoic acid (40 ul, 0.25 mmol, overnight reaction).

Octanoyl-Tyr(Bzl)-Leu-Pro-(3-(S)-guanidinomethylpyrrolidine) trifluoroacetate: Following completion of the four coupling reaction cycles, the resin was treated with 20 ml of 0.2M MeI in DMF for overnight. The MeI treatment was repeated for another two times (1 h each) to ensure complete methylation at the sulfur atom of the thio-carbonyl (C═S) units attached to resin. The resins was filtered and washed with DMF (5×3 ml) and dried overnight under vacuum. Dried resin was treated with 2M NH₄OAc dissolved in anhydrous DMSO. The reaction was carried out under argon and in a sealed tube at 80° C. for a period of 16 h. The reaction tube was cooled to room temp., and the resin was filtered and washed initially with 15 ml of DMSO (5×3 ml) followed by 15 ml (5×3 ml) of DMF solvent. Finally the resin was thoroughly washed with 20 ml (5×4 ml) MeOH and dried under vacuum for an hour. Cleavage of the peptide from the resin was achieved by treatment with a cleavage cocktail (1 ml) comprising TFA (90%), water (5%), and triethylsilane (5%) for 2 h. The cleavage cocktail was removed by filtration and retained. The resin was subjected to a second treatment with cleavage cocktail (1 ml) for another 1 h. The cleavage cocktails were combined in a small glass vial and the liquid is dried off through rotary vaporizer. Adsorbed THF and triethylsilane was removed using toluene as an azeotropic solvent. Purification of the crude peptide by semi-prep HPLC eluting with a gradient of acetonitrile in water (both 0.1% TFA) gave the pure final peptide Octanoyl-Tyr(Bzl)-Leu-Pro-(S)-(3-guanidino)pyrrolidinyl amide as a complex pair of amide rotamers: 20.2 mg (37%): ¹H NMR (d6-DMSO) δ=7.91-7.84 (m, 2H), 7.80 (m, 1H), 7.50-7.36 (m, 5H), 7.12 (d, 2H), 7.10 (br s 4H), 6.84 (d, 2H), 5.00 (s, 2H), 4.55-4.33 (m, 3H), 4.17 (m, ½H), 4.04 (m, 1H), 3.86 (m, ½H), 3.64 (m, 1½H), 3.54 (m, ½H), 3.46 (m, 1H), 3.34 (m, 1H), 3.26 (m, 1H), 3.13 (m, ½H), 2.86 (m, 1H), 2.61 (m, 1H), 2.18 (m, ½H), 2.08 (m, 1H), 1.97 (t, 2H), 1.92-1.54 (m, 5H), 1.39 (m, 2H), 1.32 (m, 2H), 1.19 (m, 2H), 1.15 (m, 3H), 1.05 (m, 2H), 0.87 (d, 3H), 0.84 (d, 3H), 0.80 (t, 3H) ppm; m/z=718.4 (M+H).

### Example 30

### Structure 30

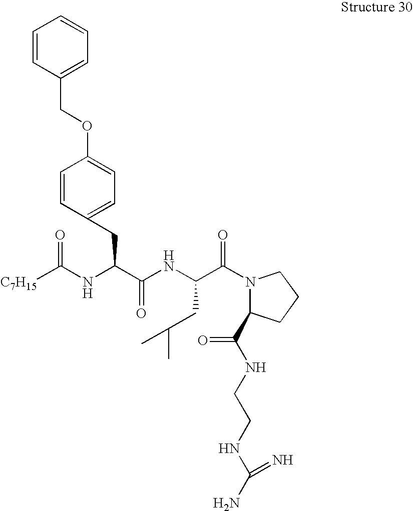

Fmoc-NH(CH₂)₂—NCS: Fmoc-NH—CH₂—CH₂—NH₂.HBr (813.5 mg, 2.24 mmol), was dissolved in 25 ml of anhydrous THF under argon at 0° C. To the stifling solution, CS₂ (1.18 ml, 19.62 mmol), DIEA (1.36 ml, 7.9 mmol), was added through syringes and the reaction was allowed for 90 min at cold temperature. Hydrogen peroxide (30% wt./vol.; 2.66 ml, 23.5 mmol), was added to the reaction product and stirring continued for an hour until the solution temperature reached at room temperature. Dilution was made with 50 ml diethyl ether and the reaction mixture was quenched with 150 ml of NaH₂PO₄ (2.07 gm, 0.1M). The organic layer was removed by separating funnel. Two subsequent extractions were made with Et₂O (100 ml) followed by washing the combined diethyl ether extract (˜300 ml) with water (100 ml) and brine solution (50 ml). Finally, the organic layer was dried over anhydrous Na₂SO₄. The resulting organic reaction mixture was dried first by rotary evaporator followed by high vacuum pump to obtained white crude solid. This solid crude was subjected to column chromatography using silica (10″×1.5″). Fractions collected using 80:20 (vol/vol) Hexane:EtOAc mixture solvent system yielded pure white powder of Fmoc-NH—(CH₂)₂—NCS (508 mg, 70%).

Coupling reaction of Fmoc-NH—(CH₂)₂—NCS with Rink Amide AM resin: The detail preparation of Fmoc deprotected Rink Amide AM resin was described previously under Structure 1: CHP-135 (110705 2.1) section. Coupling between these two reagents was achieved by reacting 395 mg (0.936 mmol) of Fmoc-NH—(CH₂)₂—NCS and Rink Amide AM resin (400 mg @0.68 mmol/g) in 3 ml anhydrous THF solution in the presence of excess base, DIEA (500 ul, 2.81 mmol) for 3 h. The resins were washed with 60 ml (6×10 ml) THF and dried under vacuum. Yield 488 mg. The resin (100 mg) was treated with 20% v/v piperidine in DMF (10 ml) for an hour, followed by filtration and subsequently washed with DMF. The procedure was repeated twice to ensure complete de protection of Fmoc moiety from terminal secondary amine unit. The resins was dried overnight under vacuum to obtain 108 mg of NH2-(CH₂)₂—NH—C(═S)—NH-AM Rink Amide resin. The resin was treated with amino acids coupling cocktail comprising an Fmoc protected aminoacids (5 eq.), PyBOP (5 eq.), HOBt (5 eq.), and DIEA (10 eq.) in 10 ml DMF and the reaction was carried out overnight. The resin was washed thoroughly with 15 ml DMF (5×3 ml) to complete one synthesis cycle followed by three separate hydrolysis reactions with 20% piperidine in DMF to free off the Fmoc group from the elongated amino acid chain except for the last cycle when octanoic acid was used as coupling unit. The Fmoc amino acids employed were consecutively, Fmoc-Pro-OH (189 mg, 0.56 mmol, overnight reaction), Fmoc-Leu-OH (168 mg, 0.53 mmol, overnight reaction), Fmoc-Tyr(Bzl)-OH (126 mg, 0.254 mmol, overnight reaction) and octanoic acid (40 ul, 0.25 mmol, overnight reaction).

Octanoyl-Tyr(Bzl)-Leu-Pro-NH(CH₂)₂ NH—C(═NH)—NH₂ trifluoroacetate: Following completion of the four coupling reaction cycles, the resin was treated with 20 ml of 0.2M MeI in DMF for 2 h. The resin was filtered, washed with 10 ml DMF (2×5 ml). The wet resin was further treated with 20 ml 0.2M MeI and the process was repeated for a third time to ensure complete methylation at the sulfur atom of the thiocarbonyl (C═S) units attached to resin. The resins was filtered and washed with DMF (5×3 ml) and dried overnight under vacuum. Dried resin was treated with 2M NH₄OAc dissolved in anhydrous DMSO. The reaction was carried out under argon and in a sealed tube at 80° C. for a period of 16 h. The reaction tube was cooled to room temp., and the resin was filtered and washed initially with 15 ml of DMSO (5×3 ml) followed by 15 ml (5×3 ml) of DMF solvent. Finally the resin was thoroughly washed with 20 ml (5×4 ml) MeOH and dried under vacuum for an hour. Cleavage of the peptide from the resin was achieved by treatment with a cleavage cocktail (1 ml) comprising TFA (90%), water (5%), and triethylsilane (5%) for 2 h. The cleavage cocktail was removed by filtration and retained. The resin was subjected to a second treatment with cleavage cocktail (1 ml) for another 1 h. The cleavage cocktails were combined in a small glass vial and the liquid is dried off through rotary vaporizer. Adsorbed THF and triethylsilane was removed using toluene as an azeotropic solvent. Purification of the crude peptide by semi-prep HPLC eluting with a gradient of acetonitrile in water (both 0.1% TFA) gave the pure final peptide: 9.4 mg (14%): ¹H NMR (d6-DMSO) δ=7.99 (d, 1H), 7.97 (d, 1H), 7.87 (d, 1H), 7.40-7.27 (m, 6H), 7.10 (d, 2H), 6.84 (d, 2H), 5.01 (s, 2H), 4.50 (m, 1H), 4.46 (m, 1H), 4.20 (dd, 1H), 3.58 (m, 1H), 3.44 (m, 1H), 3.27 (m, 1H), 3.19 (m, 1H), 3.12 (m, 2H), 2.84 (dd, 1H), 2.59 (dd, 1H), 2.01 (m, 1H), 1.97 (t, 2H), 1.91 (m, 1H), 1.81 (m, 1H), 1.77 (m, 1H), 1.60 (m, 1H), 1.41 (m, 2H), 1.32 (m, 2H), 1.25-1.10 (m, 6H), 1.04 (m, 2H), 0.86 (m, 6H), 0.81 (t, 3H) ppm; m/z=692.4 (M+H).

### Example 31

### Structure 31

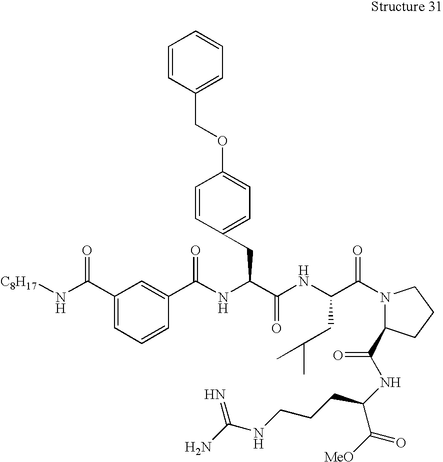

3-(Octylamido)-nicotinoyl-Tyr(Bzl)-Leu-Pro-OMe: Boc-Tyr(Bzl)-Leu-Pro-OMe (250 mg, 0.42 mmol) was dissolved in CH₂Cl₂ (4 mL) and TFA (4 mL) added dropwise. After 35 mM the volatile components were removed on a rotary evaporator and the residual oil placed under vacuum for 30 min. The resulting glass was dissolved in dimethylformamide (5 mL) and 3-(octylamido)-nicotinic acid (1 eq, 146 mg) added. BOP (1.0 eq., 186 mg) and then diisopropylethylamine (DIEA) (6 eq., 325 mg, 440 μL) was added with stirring and the mixture stirred for 17 h. The reaction mixture was quenched by addition to water (80 mL). Sodium chloride (1 gm) was added. The resulting mixture was extracted with ethyl acetate (100 mL) and the organic phase washed with 0.1N aqueous potassium hydroxide (2×30 mL), water (2×30 mL) and dried over sodium sulfate. Evaporation of the solvent gave a tan solid which was dried under vacuum to give an impure tan solid (359 mg, 113%). The sample was used without further purification.

3-(Octylamido)-nicotinoyl-Tyr(Bzl)-Leu-Pro-OH: Octanoyl-Tyr(Bzl)-Leu-Pro-OMe (309 mg, 0.409 mmol) was dissolved in THF (15 mL) and water (15 mL) added dropwise with stifling. Lithium hydroxide monohydrate (12 eq, 206 mg) and methanol (1 mL) was added and stifling continued for 4 h. The volatile solvents were removed on a rotary evaporator and the remaining aqueous solution diluted to 35 mL with water. 2N Hydrochloric acid (12 eq, 4.91 mL)) was added dropwise and stifling was continued for 30 min. The resulting off-white solid was collected by filtration, and washed with water (4×5 mL). Drying under vacuum gave the desired carboxylic acid (285 mg, 94%). The sample was used without further purification.

3-(Octylamido)-nicotinoyl-Tyr(Bzl)-Leu-Pro-Arg(Pbf)-OMe: 3-(Octylamido)-nicotinoyl-Tyr(Bzl)-Leu-Pro-OH (240 mg, 0.323 mmol) was dissolved in DMF (4 mL) and H-Arg(Pbf)-OMe hydrochloride (1 eq, 154 mg) and BOP (1 eq. 143 mg) were added with stirring. DIEA (6 eq., 1438 mg, 1.94 mL) was added, and the reaction mixture stirred for 18 h. The resultant mixture was quenched by addition to water (75 mL) and stirred for 1 h. Sodium chloride (˜0.5 g) added and stirring continued for 0.5 h to give a fine tan precipitate. The solid was collected by filtration, and washed with water (4×10 mL). Drying under vacuum gave the desired ester (351 mg, 93%). The sample was used without further purification.

3-(Octylamido)-nicotinoyl-Tyr(Bzl)-Leu-Pro-Arg-OMe trifluoroacetate: 3-(Octanoyl)-nicotinoyl-Tyr(Bzl)-Leu-Pro-Arg(Pbf)-OMe (60 mg, 0.0515 mmol) was dissolved in TFA (2 mL) and triiopropylsilane (xs, 5 drops) added. The reaction mixture was stirred for 1.5 h, evaporated to dryness and purified by semi-prep HPLC gave the desired deprotected tetrapeptide ester trifluoroacetate salt as a white solid (23.5 mg, 45%): ¹H NMR (d6-DMSO) δ=9.04 (d, 1H), 8.96 (d, 1H), 8.83 (d, 1H), 8.70 (t, 1H), 8.47 (m, 1H), 8.24 (d, 1H), 8.20 (d, 1H), 7.42 (m, 1H), 7.39-7.28 (m, 5H), 7.22 (d, 2H), 6.86 (d, 2H), 4.98 (s, 2H), 4.70 (m, 1H), 4.53 (m, 1H), 4.33 (m, 1H), 4.19 (m, 1H), 3.63 (m, 1H), 3.59 (s, 3H), 3.46 (m, 2H), 3.22 (m, 2H), 3.06 (m, 2H), 3.00 (m, 1H), 2.84 (m, 1H), 2.02 (m, 1H), 1.90-1.36 (m, 11H), 1.36-1.16 (m, 11H), 0.86 (m, 6H), 0.82 (t, 3H) ppm; m/z=912.5 (M+H).

### Example 32

### Structure 32

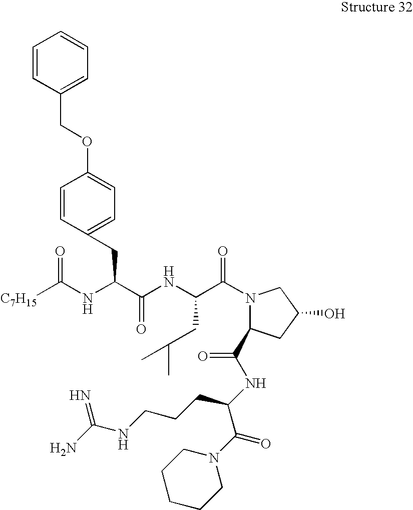

Boc-Tyr(Bzl)-Leu-OMe: Boc-Tyr(Bzl)-OH 744 mg, 2.0 mmol) and H-Leu-OMe hydrochloride (364 mg, 2.0 mmol) was dissolved in dimethylformamide (10 mL). BOP (1.0 eq., 884 mg) and then diisopropylethylamine (DIEA) (7 eq., 1806 mg, 2.43 mL) was added with stirring and the mixture stirred for 21 h. The reaction mixture was quenched by addition to water (200 mL) and stirred for 0.5 h. The resultant white precipitate was collected by filtration and washed with water (5×50 mL). The remaining solid was dried under vacuum to give a white solid (956 mg, 96%). The sample was used without further purification.

Octanoyl-Tyr(Bzl)-Leu-OMe: Boc-Tyr(Bzl)-Leu-OMe (249 mg, 0.5 mmol) was dissolved in CH₂Cl₂ (2.5 mL) and TFA (0.52 mL) added dropwise. After 30 min the volatile components were removed on a rotary evaporator and the residual oil placed under vacuum for 2 h. The resulting glass was dissolved in dimethylformamide (5 mL) and octanoic acid (1 eq, 72 mg, 80 μL) added. BOP (1.0 eq., 221 mg) and then diisopropylethylamine (DIEA) (6 eq., 387 mg, 521 μL) was added with stifling and the mixture stirred for 17 h. The reaction mixture was quenched by addition to water (80 mL) and stirred for 2 h. The resultant white precipitate was collected by filtration and washed with water (4×10 mL). The remaining solid was dried under vacuum to give a white solid (231 mg, 88%). The sample was used without further purification.

Octanoyl-Tyr(Bzl)-Leu-OH: Octanoyl-Tyr(Bzl)-Leu-OMe (131 mg, 0.25 mmol) was dissolved in THF (5 mL) and water (3.5 mL) added dropwise with stirring. Lithium hydroxide monohydrate (12 eq, 126 mg) was added and stirring continued for 4 h. The THF was removed on a rotary evaporator and the remaining aqueous solution diluted to 10 mL with water. 2N Hydrochloric acid (24 eq, 3 mL)) was added dropwise and stifling was continued for 0.5 h. The resulting white solid was collected by filtration, and washed with water (5×10 mL). Drying under vacuum gave the desired carboxylic acid (125 mg, 98%). The sample was used without further purification.

Octanoyl-Tyr(Bzl)-Leu-Hyp-OMe: Boc-Hyp-OMe (61.3 mg, 0.25 mmol) was dissolved in CH₂Cl₂ (1.25 mL) and TFA (1.25 mL) added dropwise. After 45 min the volatile components were removed on a rotary evaporator and the residual oil placed under vacuum for 2 h. The resulting glass was dissolved in dimethylformamide (2.5 mL) and Octanoyl-Tyr(Bzl)-Leu-OH (1 eq., 125 mg) was added. BOP (1 eq. 111 mg) were added with stirring. DIEA (6 eq., 194 mg, 260 μL) was added, and the reaction mixture stirred for 18 h. The resultant mixture was quenched by addition to water (40 mL) and stirred for 1 h. Sodium chloride (˜0.5 g) added and stirring continued for 0.5 h. The resultant mixture was extracted with ethyl acetate (40 mL and 30 mL). The combined organic phases were washed with water (4×60 mL) and brine (30 mL) and dried over sodium sulfate. Evaporation of the solvent left a white solid (143 mg, 90%). The sample was used without further purification.

Octanoyl-Tyr(Bzl)-Leu-Hyp-OH: Octanoyl-Tyr(Bzl)-Leu-Hyp-OMe (143 mg, 0.22 mmol) was dissolved in THF (5 mL) and water (4 mL) added dropwise with stifling. Lithium hydroxide monohydrate (12 eq, 113 mg) was added and stirring continued for 4 h. The THF was removed on a rotary evaporator and the remaining aqueous solution diluted to 14 mL with water. 2N Hydrochloric acid (24 eq, 2.65 mL)) was added dropwise and stirring was continued for 1 h. The resultant mixture was extracted with ethyl acetate (2×20 mL). The combined organic phases were washed with water (3×20 mL) and brine (20 mL) and dried over sodium sulfate. Evaporation of the solvent left a tan solid (90 mg, 64%). The sample was used without further purification.

Octanoyl-Tyr(Bzl)-Leu-Hyp-Arg(Boc)₂N(CH₂)₅: Octanoyl-Tyr(Bzl)-Leu-Pro-OH (31.1 mg, 0.05 mmol) was dissolved in DMF (0.5 mL) and H-Arg(Boc)₂N(CH₂)₅ trifluoroacetate salt (1 eq, 28 mg) and BOP (1 eq. 22.1 mg) were added with stirring. DIEA (7 eq., 50 mg, 67 μL) was added, and the reaction mixture stirred for 19 h. The resultant mixture was quenched by addition to water (12 mL) and stirred for 2 h. Sodium chloride (˜0.25 g) added and stifling continued for 1 h to give a fine tan precipitate. The solid was collected by filtration, and washed with water (6×5 mL). Drying under vacuum gave the desired piperidinyl amide (44 mg, 84%). The sample was used without further purification.

Octanoyl-Tyr(Bzl)-Leu-Hyp-ArgN(CH₂)₅ trifluoroacetate: Octanoyl-Tyr(Bzl)-Leu-Hyp-Arg(Boc)₂N(CH₂)₅ (43 mg, 0.04 mmol) was dissolved in dichloromethane (0.5 mL) and trifluoroacetic acid (0.5 mL) added. The reaction mixture was stirred for 45 min, evaporated to dryness and purified by semi-prep HPLC gave the desired fully deprotected tetrapeptide amide trifluoroacetate salt as a white solid (19 mg, 48%): ¹H NMR (d6-DMSO) δ=8.18 (m, 1H), 8.13 (d, ½H), 7.98 (d, ½H), 7.90 (m, 1H), 7.43-7.26 (m, 6H), 7.16 (d, 1H), 7.11 (d, 1H), 6.86 (d, ½H), 6.85 (d, ½H), 5.08 (dd, 1H), 5.00 (s, 2H), 4.61 (m, 1H), 4.53 (m, 1H), 4.45 (m, ½H), 4.39 (t, ½H), 4.30 (1H), 3.57 (m, 1H), 3.52-3.30 (m, 6H), 3.06 (m, 2H), 2.86 (m, 1H), 2.61 (m, 1H), 2.00 (m, 1H), 1.97 (t, 2H), 1.80 (m, 1H), 1.66-1.26 (m, 15H), 1.26-1.08 (m, 6H), 1.04 (m, 2H), 0.88-0.79 (m, 9H) ppm; m/z=847.6 (M+H).

### Example 33

### Structure 33

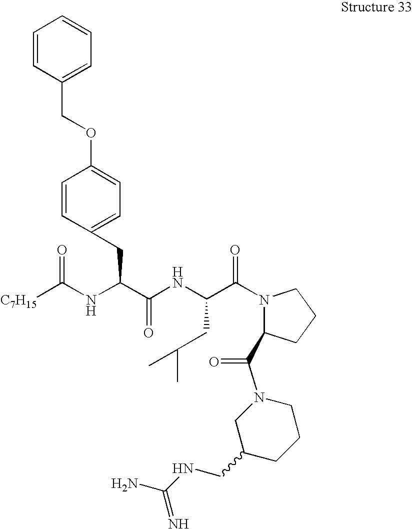

Octanoyl-Tyr(Bzl)-Leu-Pro-(3-aminomethylpiperidine) trifluoroacetate: Octanoyl-Tyr(Bzl)-Leu-Pro-OH (125 mg, 0.206 mmol) was dissolved in 2.1 ml DMF. To the stirring solution, solid (±)-3-(Boc-aminomethyl)piperidine (48.6 mg, 0.227 mmol), BOP (99 mg, 0.224 mmol), and then diisopropylethylamine (DIEA) (110 ul, 1.24 mmol) was added. The reaction mixture was allowed to stir for 16 h at room temperature. The reaction mixture was quenched by addition to water (40 ml) followed by addition of 700 mg NaCl to achieve maximum precipitation of grayish white solid. The crude precipitate was separated by filtration and washed thoroughly with water (5×5 ml) and dried over night under high vacuum. The intermediate product was subjected to acid hydrolysis reaction using 1:1 (v/v) trifluoroacetic acid and dichloromethane mixture for 45 min. The organic volatile components were removed by rotary vacuum and the adsorbed TFA was removed using azeotropic mixture with toluene as co-solvent. The brown mass was dried overnight under high vacuum to provide the desired salt (140 mg, yield 98%).

Octanoyl-Tyr(Bzl)-Leu-Pro-(3-guanidinomethylpiperidine) trifluoroacetate: Octanoyl-Tyr(Bzl)-Leu-Pro-(3-aminomethylpiperidine) trifluoroacetate (140 mg, 0.202 mmol) was dissolved in THF (3 mL) and to it solid N,N′-Bis(Boc)-1H-pyrazole-1-carboxamidine (60 mg, 0.183 mmol), was added followed by the addition of diisopropylethylamine (DIEA) (5 eq., 160 ul, 0.919 mmol) and 1 ml of H₂O. The reaction mixture was allowed to stir for 60 h at room temperature. After completion, the organic volatiles were removed under vacuum and dried to give the title compound as a yellow solid. The yellow solid was dissolved in TFA:CH₂Cl₂ (1:1; v/v) (20 mL) and allow to stand for 1 h. The organic volatile components were removed and dried azeotropically using toluene as co-solvent. The crude product was purified by semi-prep HPLC eluting with a gradient of acetonitrile in water (both contain 0.1% TFA) to give the desired material as a mixture of two diastereomers (118 mg, 78%): ¹H NMR (d6-DMSO) δ=7.93-7.85 (m, 2H), 7.58 (d, ½H), 7.48 (d, ½H), 7.41-7.27 (m, 5H), 7.11 (d, 1H), 7.10 (d, 1H), 6.84 (d, 2H), 5.01 (s, 2H), 4.76 (m, 1H), 4.52 (m, 1H), 4.44 (m, 1H), 4.06 (m, ½H), 3.91 (m, ½H), 3.78 (m, ½H), 3.69 (m, ½H), 3.64 (m, ½H), 3.43 (m, ½H), 3.21 (m, 1H), 3.10-2.55 (m, 5H), 2.09 (m, 1H), 1.97 (t, 2H), 1.92-1.50 (m, 5H), 1.48-1.26 (m, 4H), 1.26-1.09 (m, 8H), 1.04 (m, 2H), 0.86 (m, 6H), 0.81 (t, 3H) ppm; m/z=746.6 (M+H).

### Example 34

### Structure 34

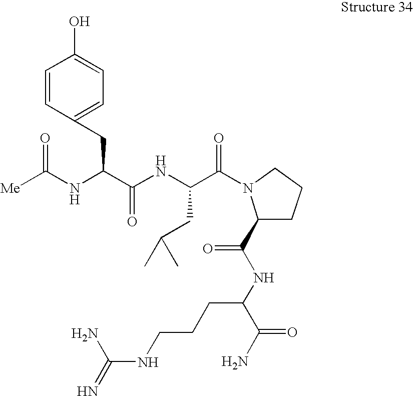

### Biological Assays

After synthesis, the resulting peptides are screened for antibiotic efficacy. In one embodiment, the synthesized peptide may be screened through DnaK inhibition in vitro assays in bacteria, such as but not limited to H. influenzae, as described in the examples below.

Inhibition of the bacterial DnaK chaperone system may be evaluated by inhibition of the DnaK induced refolding of denatured firefly luciferase. More specifically, firefly luciferase is denatured by treatment with 8N guanidium hydrochloride at room temperature for 30 mins and then 5° C. for 1 h. After dilution into cold MOPS buffer augmented with ATP (2.5 mM) and DTT (12.5 mM) and standing for a further hour, the denatured luciferase is refolded by addition to a mixture of chaperone proteins in MOPS buffer, resulting in a reaction mixture containing denatured luciferase (24 nM), DnaK (200 nM), DnaJ (100 nM) and GrpE (400 nM). The extent of refolding is determined by addition of a luciferase substrate and measurement of the emitted chemoluminescence. In this format the substrate is the commercially available luciferase “Steady-Glo” reagent from Promega, Inc. Inhibition of DnaK induced refolding is determined by premixing the chaperone proteins with the test agent at concentrations of test agent ranging from 1-100 μg/mL. The extent of refolding is compared to that with no test reagent, and the IC₅₀ is estimated by fitting the data to a standard sigmoidal dose-response curve.

An in vitro assay may be conducted by determining the minimum inhibitory concentration (MIC) of the small molecules to Gram negative bacteria, such as H. influenza. H. influenzae MICs are determined using CLSI recommended broth microdilution methodology (M7-A7). Test agent powders are dissolved in DMSO, serial dilutions are also made in DMSO and 1:100 dilutions of each are then prepared in Haemophilus Test media (HTM). 100 μL of each final dilution are transferred to appropriate wells of a 96 well microtiter plate. The test organism is suspended in saline or Mueller Hinton broth to an organism density equivalent to a 0.5 McFarland Standard. 300 μL of the H. influenzae suspension is transferred to 11 mLs of HTM and 10 μL of this suspension is transferred to each well of the microtiter plate. Plates are incubated under ambient conditions at 35° C. for 20-24 hours and the MIC is read as the lowest drug concentration showing no visible growth. Colony counts are determined for each inoculum and expected range of final organism concentration is 3-7×10⁵ CFU/mL. One of the recommended H. influenzae quality control strains (ATCC 49247 or 49766) is also tested on each testing day.

The methods of testing the DnaK capacity of the small molecules of the present invention are not limited to the above. Rather any similar method understood in the art may be utilized to test the effectiveness of the desired molecule and any similar organism may be used, such as those Gram-negative organisms discussed herein.

### In Vitro DnaK Inhibiting Activity of the Peptide Structures

Inhibition of the bacterial DnaK chaperone system was evaluated by inhibition of the DnaK induced refolding of denatured firefly luciferase. Firefly luciferase was denatured by treatment with 8N guanidium hydrochloride at room temperature for 30 mins and then 5° C. for 1 h. After dilution into cold MOPS buffer augmented with ATP (2.5 mM) and DTT (12.5 mM) and standing for a further hour, the denatured luciferase was refolded by addition to a mixture of chaperone proteins in MOPS buffer, resulting in a reaction mixture containing denatured luciferase (24 nM), DnaK (200 nM), DnaJ (100 nM) and GrpE (400 nM). The extent of refolding was determined by addition of a luciferase substrate and measurement of the emitted chemoluminescence. In this format the substrate is the commercially available luciferase “Steady-Glo” reagent from Promega, Inc. Inhibition of DnaK induced refolding was determined by premixing the chaperone proteins with the test agent at concentrations of test agent ranging from 1-100 μg/mL. The extent of refolding was compared to that with no test reagent, and the IC₅₀ was estimated by fitting the data to a standard sigmoidal dose-response curve.

Table 2 reports the inhibitory concentrations (IC₅₀) of Structures #1 to #34 identified in Examples against DnaK, which demonstrate the ability of the compounds to bind to the DnaK binding domain that facilitates protein folding by measuring the inhibition of the known ability of DnaK to induce refolding of denatured firefly luciferase. IC₅₀ is defined as the concentration in μM at which 50% inhibition of DnaK induced refolding of denatured firefly luciferase occurs.

TABLE 2
DnaK Inhibition Assay

| Structure | DnaK IC₅₀ (μM) | DnaK IC₅₀(μg/ml) |
|---|---|---|
| pyrrhocoricin | 29 | 82 |
| 1 | 58 | 46 |
| 2 | 63 | 118 |
| 3 | 110 | 209 |
| 4 | 3 | 4 |
| 5 | 45 | 63 |
| 6 | 147 | 244 |
| 7 | 72 | 127 |
| 8 | 30 | 61 |
| 9 | 62 | 132 |
| 10 | 26 | 57 |
| 11 | 69 | 158 |
| 12 | 11 | 11 |
| 13 | 12 | 17 |
| 14 | 25 | 28 |
| 15 | 24 | 29 |
| 16 | 24 | 26 |
| 17 | 21 | 19 |
| 18 | 54 | 55 |
| 19 | 40 | 46 |
| 20 | 16 | 17 |
| 21 | 22 | 21 |
| 22 | 14 | 27 |
| 23 | 101 | 232 |
| 24 | 240 | 131 |
| 25 | 20 | 18 |
| 26 | 30 | 27 |
| 27 | 8 | 7 |
| 28 | 13 | 12 |
| 29 | 10 | 8 |
| 30 | 19 | 15 |
| 31 | 10 | 10 |
| 32 | 12 | 12 |
| 33 | 23 | 20 |

The results of Table 2 demonstrate the inhibitory potency of the polypeptides of this invention. Generally, the Structures identified inhibited DnaK at a comparable molar concentration to that of the control, thus exhibiting higher potency than that of unmodified pyrrhocoricin in a μg/mL comparison. Accordingly, specific embodiments of this invention demonstrate greater inhibition of E. coli DnaK than unmodified pyrrhocoricin.

### Bacterial Growth Inhibition

H. influenzae MICs are determined using CLSI recommended broth microdilution methodology (M7-A7). Test agent powders are dissolved in DMSO, serial dilutions are also made in DMSO and 1:100 dilutions of each are then prepared in Haemophilus Test media (HTM). 100 μL of each final dilution are transferred to appropriate wells of a 96 well microtiter plate. The test organism is suspended in saline or Mueller Hinton broth to an organism density equivalent to a 0.5 McFarland Standard. 300 μL of the H. influenzae suspension is transferred to 11 mLs of HTM and 10 μL of this suspension is transferred to each well of the microtiter plate. Plates are incubated under ambient conditions at 35° C. for 20-24 hours and the MIC is read as the lowest drug concentration showing no visible growth. Colony counts are determined for each inoculum and expected range of final organism concentration is 3-7×10⁵ CFU/mL. One of the recommended H. influenzae quality control strains (ATCC 49247 or 49766) is also tested on each testing day.

Table 3 reports the minimum inhibitory concentration (MIC) of Structures 2, 3, 10, 11, and 22 identified above against two strains of the Gram-negative bacteria H. influenzae. Pyrrhocoricin was used as a control. MIC is defined as the lowest concentration of an anti-bacterial agent that will inhibit the visible growth of a microorganism after overnight incubation.

TABLE 3
H. influenzae Inhibition Assay

| | H. influenzae (Strain 109) | H. influenzae (Strain 110) |
|---|---|---|
| Structure | MIC (μg/ml) | MIC (μg/ml) |
| pyrrhocoricin | >128 | >128 |
| 2 | 32 | >128 |
| 3 | 32 | 64 |
| 10 | 32 | >128 |
| 11 | 4 | >128 |
| 22 | 2 | >128 |

The results of Table 2 demonstrate the inhibitory potency of Structures 2, 3, 10, 11, and 22. Structures 2, 10, 11, and 22 inhibited H. influenzae strain 109 at a lower concentration than the control, thus surpassing the potency of pyrrhocoricin against strain 109 of H. influenzae. The potency of Structure 3 surpasses that of pyrrhocoricin against both strains 109 and 110 of H. influenzae. Accordingly, specific embodiments of this invention exhibit greater antibacterial efficacy than unmodified pyrrhocoricin.

The results of Table 4 demonstrate the antibacterial activity of Structures 25-34 against E. Coli.

TABLE 4
Antibacterial activity of Selected Examples against E. coli

| | E. coli (ATCC 25922) |
|---|---|
| Example # | MIC (μg/mL) |
| 25 | 128 |
| 26 | 32 |
| 27 | NT |
| 28 | 32 |
| 29 | 32 |
| 30 | 32 |
| 31 | 32 |
| 32 | 64 |
| 33 | 16 |
| 34 | >256 |

### Utility of the Invention

HSP70 has gained attention as a putative target for tumor therapy. (Mikkel Rohde et al., 2005, GENES & DEVELOPMENT 19:570-582.) The present invention provides small molecule inhibitors of mammalian HSP70 proteins and the use of such species for the therapeutic treatment of cancer.

Compositions of the present invention, as noted above, are targeted to treat infection of a mammal, e.g. a human, by a bacterium. While the present invention may be used to inhibit chaperone activity of bacterial homologs of HSP70 within any strain of bacteria, in one non-limiting embodiment, the bacterium may be a Gram negative bacteria. Gram-negative bacterial organisms include, but are not limited to, organisms of the following genera: Achromobacter, Acidaminococcus, Acidovorax, Acinetobacter, Actinobacillus, Aeromonas, Alcaligenes, Aminobacter, Anaerobiosprillum, Anaerorhabdus, Anaerovibrio, Ancylobacter, Aquaspirillum, Bacteroides, Bergeyella, Bilophila, Bordetella, Brenneria, Brevundimonas, Brucella, Budvicia, Burkholderia, Buttiauxella, Butyrivibrio, Campylobacter, Capnocytophaga, Cardiobacterium, Catonella, Cedecea, Centipeda, Chromobacterium, Chryseobacterium, Chryseomonas, Citrobacter, Comamonas, Cytophaga, Delftia, Desulfomonas, Desulfovicrio, Dialister, Dichelobacter, Edwardsiella, Eikenella, Empedobacter, Erwinia, Escherichia, Ewingella, Fibrobacter, Flavimonas, Flavobacterium, Francisella, Fusobacterium, Gilardi, Haemophilus, Hafnia, Herbaspirillum, Hydrogenophaga, Iodobacter, Janthinobacterium, Johnsonella, Kingella, Klebsiella, Kluyvera, Lampropedia, Leclercia, Leminorella, Leptotrichia, Listonella, Mannheimia, Megamonas, Megasphaera Mitsuokella, Moellerella, Moraxella, Morganella, Myroides, Neisseria, Obesumbacterium, Oceanomonas, Ochrobactrum, Oligella, Ornithobacterium, Pandoraea, Pantoea, Pasteurella, Paucimonas, Pectobacterium, Pedobacter, Photobacterium, Photorhabdus, Phyllobacterium, Plesiomonas, Porphyromonas, Pragia, Prevotella, Proteeae, Proteus, Providencia, Pseudomonas, Psychrobacter, Rahnella, Ralstonia, Raoultella, Rhizobium, Riemerella, Rikenella, Roseomonas, Ruminobacter Salmonella, Sebaldella, Selenomonas, Serpens, Serratia, Shewanella, Shigella, Simonsiella, Sinorhizobium, Sphingobacterium, Sphingomonas, Stenotrophomonas, Succinivibrio, Succinimonas, Sutterella, Suttonella, Tatumella, Taylorella, Tissierella, Trabulsiella, Variovorax, Veillonella, Vibrio, Vogesella, Weeksella, Xanthomonas, Xenorhabdus, Yersinia, and Yokenella. This list of genera is not intended to be limiting and the may include any Gram-negative genus or species discussed within Murray et al. Manual of Clinical Microbiology, the contents of which are incorporated herein by reference, or any other Gram-negative bacterial genus and/or species known in the art.

The above Gram-negative bacteria is not intended to limit the present invention. Rather the present invention is intended to inhibit the chaperone activity of all homologs of HSP70. To this end, the present invention may be used to treat infections caused by Gram-positive bacteria or any other microorganism which may utilize a homolog of an HSP70 chaperone protein, for example, Straph, Strep, Enterococci, etc.

The small molecules of the present invention may be administered along with other active agents, such as conventional antibiotics. Non-limiting examples of antibiotics include, tetracyclines, penicillins, cephalosporins, carbopenems, aminoglycosides, macrolide antibiotics, lincosamine antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, Gramicidin, mecillinam, cloxiquine, chlorhexidine, dichlorobenzylalcohol, methyl-2-pentylphenol and any combination thereof. The above listed antibiotics are not intended to be limiting. As such, an antibiotic of the present invention may include any antibiotic listed in any current or previous Physicians' Desk Reference.

It is desirable that coadministration of the antibiotics or other active agents enhances the antibacterial potential of the small molecules of the present invention, the known antibacterial agent, or both. Such coadministration to patients suffering from a bacterial infection has the possible benefit of i) lowering the necessary therapeutically effective dose; ii) extending the duration of activity of a fixed dose; iii) reducing the likelihood of the development of resistant strains of the infecting organism; and/or iv) expanding the spectrum of activity of the individual agents. Agents that exhibit a therapeutic synergistic effect when coadministered are preferred. As used herein, the terms “synergy” or “synergistic” refer to the combined effect of administering two therapeutic agents, where the overall response is greater than the sum of the two individual effects. The term synergy also refers to the combined effect of administering an amount of one therapeutic agent that, when administered as monotherapy, produces minimal measurable response but, when administered in combination with another therapeutic compound, produces an overall response that is greater than that produced by the second compound alone.

One or more of the small molecules, or pharmaceutical salts thereof, of the present invention may be formulated into an anti-bacterial composition with an optional additional active ingredient, e.g. antibiotic. The composition may be within a pharmaceutically acceptable carrier and may include other optional components. As pharmaceutical compositions, the composition of the present invention may be admixed with a pharmaceutically acceptable vehicle or carrier suitable for administration as a protein composition. These peptides may be combined in a single pharmaceutical preparation for administration. Suitable pharmaceutically acceptable carriers for use in a pharmaceutical proteinaceous composition of the invention are well known to those of skill in the art. Such carriers include, for example, saline, buffered saline, liposomes, oil in water emulsions, vegetable oil, ethanol, polyol (e.g. glycerol, propylene glycol, liquid polyethylene glycol, etc.) and the like. The compositions may further include a detergent to make the peptide more bioavailable, e.g., octylglucoside. To this end, the present invention is not limited by the selection of the carrier or detergent and may include any carrier known in the art to administer an anti-microbial agent to a mammal. The formulation may also include diluents as well as, in some cases, adjuvants, buffers, preservatives and the like.

The pharmaceutical compositions may also be formulated to suit a selected route of administration, and may contain ingredients specific to the route of administration. Routes of administration of such pharmaceutical compositions are usually split into five general groups: inhaled, oral, transdermal, parenteral and suppository. In the present invention, any of these routes may be utilized so long as the peptide or variant of the present invention is placed into contact with the targeted bacteria, such as by way of the blood stream of the mammal. In one embodiment, the pharmaceutical compositions of the present invention may be suited for parenteral administration by way of injection. To this end, the injection may be intravenous, intradermal, intramuscular, or subcutaneous. Alternatively, the composition of the present invention may be formulated for oral administration. To this end, the pharmaceutical composition of the present invention may be a pill, tablet capsule, solution, syrup, elixir, suspension, gel, or powder.

A method of treating a mammalian bacterial infection involves administering to an infected mammal an effective anti-bacterial amount of a small molecule, or pharmaceutically acceptable salt thereof, of the present invention. In one embodiment, the small molecule, or pharmaceutical salt thereof, may be administered as a pharmaceutical composition described above. In one embodiment, the method of use of the present invention is such that administration of the small molecule, pharmaceutical salt thereof, or composition of the present invention ultimately leads to the treatment of infection caused by bacteria, such as a Gram negative bacterium. The dosage of the small molecules of the present invention in each anti-bacterial effective dose is selected with regard to consideration of the pathogen causing the infection, the severity of infection, the patient's age, weight, sex, general physical condition and the like. The amount of active component required to induce an effective anti-bacterial or anti-fungal effect without significant adverse side effects varies depending upon the pharmaceutical composition employed and the optional presence of other components, e.g., antibiotics and the like. The small molecules, or pharmaceutical salt thereof, of the present invention, or compositions thereof, will generally be used in an amount effective to achieve the intended purpose. To this end, dosages of the small molecules, or pharmaceutical salt thereof, will depend on the particular application. For example, for use to treat or prevent microbial infections or diseases related thereto, the small molecules of the present invention, or compositions thereof, are administered or applied in a therapeutically effective amount. By therapeutically effective amount is meant an amount effective to ameliorate the symptoms of, or ameliorate, treat or prevent microbial infections or diseases related thereto. Determination of a therapeutically effective amount is well within the capabilities of those skilled in the art, especially in light of the detailed disclosure and examples provided herein.

For systemic administration, a therapeutically effective dose can be estimated initially from in vitro assays. For example, a dose can be formulated in animal models to achieve a circulating peptide concentration range that includes the IC50 as determined in cell culture (i.e., the concentration of test compound that is lethal to 50% of a cell culture), the MIC, as determined in cell culture (i.e., the minimal inhibitory concentration for growth) or the IC100 as determined in cell culture (i.e., the concentration of peptide that is lethal to 100% of a cell culture). Such information can be used to more accurately determine useful doses in humans.

Initial dosages can also be estimated from in vivo data, e.g., animal models, using techniques that are well known in the art. One having ordinary skill in the art could readily optimize administration to humans based on animal data. Based on this information, one may administer the small molecules, or compositions thereof, in single or multiple doses each day. The antimicrobial therapy may be repeated intermittently while infections are detectable or even when they are not detectable. Additionally, as provided above, the therapy may be provided alone or in combination with other drugs, such as for example antibiotics or other antimicrobial peptides.

In cases of local administration or selective uptake, the effective local concentration of peptide may not be related to plasma concentration. One having skill in the art will be able to optimize therapeutically effective local dosages without undue experimentation.

The amount of peptide administered will be dependent on the subject being treated, on the subject's weight, the severity of the affliction, the manner of administration and the judgment of the prescribing physician.

All publications cited in the specification, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A compound of Formula I: 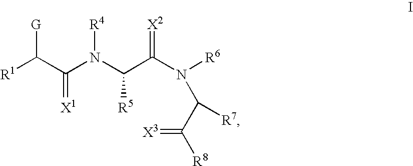
wherein
(a) X¹, X², X³ are identical or different and are independently selected from the group consisting of an oxygen atom, a sulfur atom, and two hydrogen atoms;
(b) R¹ is a substituent group selected from the group consisting of a hydrogen, 1 to 16 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 10 carbon hydroxyalkyl group, a 1 to 6 carbon alkylamino group, a 1 to 6 carbon dialkylamino group, a 1 to 6 carbon aminoalkyl group, a 1 to 6 carbon alkoxyamino group, or a substituent group defined by Formula II(a): 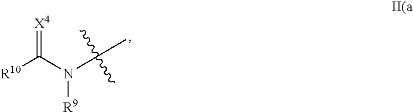
wherein R⁹ is a substituent group selected from the group consisting of a hydrogen atom and a 1 to 6 carbon alkyl; R¹⁰ is a substituent group selected from the group consisting of a hydroxyl group, a hydroxyalkyl, a 1 to 17 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 6 carbon alkylamino group and a 1 to 6 carbon dialkylamino group, and an aminoalkyl group; and X⁴ is a substituent group selected from the group consisting of an oxygen atom, a sulfur atom, and two hydrogen atoms;
(c) G is a substituent group selected from the group consisting of a benzyl group, a substituted benzyl group, a phenyl group, a substituted phenyl group, a heterocyclic aromatic alkyl group, a 1 to 6 carbon alkyl group, and a substituted 1 to 6 carbon alkyl group;
(d) R⁴ is a substituent group selected from the group consisting of a hydrogen atom, a 1 to 6 carbon alkyl group, and a substituent group forming a ring with a G substituent group;
(e) R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group;
(f) R⁶ is a substituent group selected from the group consisting of a hydrogen atom, a 1 to 6 carbon alkyl group, and a substituent group forming a ring with R⁷;
(g) R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group; and
(h) R⁸ is a substituent group which is selected from the group consisting of a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a secondary amine, a tertiary amine, a tertiary cyclic amine optionally substituted by a guanidinylalkyl group, a 1 to n carbon aminoalkylamino group, a 1 to n carbon aminoalkylamide group, a 1 to n carbon aminoalkyl-carbamate group, a 1 to n carbon aminoalkylurea group, a 1 to n carbon aminoalkylamidine group, a 1 to n carbon aminoalkylguanidine, where n=2-6, and a peptide moiety comprised of 1-4 amino acids bound at the R⁸ position by an N-terminus end of the peptide moiety where a plurality of additional substituent groups extend from an N terminus, a side chain and a C terminus of each amino acid of the peptide moiety wherein each of the plurality of additional substituent groups may be independently selected from the group consisting of a hydrogen atom, a hydroxyl group, a heterocyclic aromatic compound, an alkyl ester group, a cycloalkyl group, an amino alkyl group, an aminoalkylamino group, a primary amine, a secondary amine, and a tertiary amine.

2. The compound of claim 1 wherein the R¹ substituent group is a hydrogen atom.

3. The compound of claim 1 wherein the R¹ substituent group is represented by the structure of Formula II 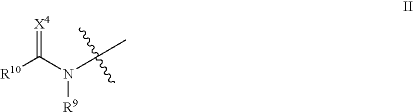
wherein X⁴ is an oxygen atom, R⁹ is a hydrogen atom and R¹⁰ is selected from the group consisting of a methyl group and a tert-butoxy group.

4. The compound of claim 1 wherein the G substituent group is a sec-butyl group.

5. The compound of claim 1 wherein the G substituent group is a substituted benzyl group.

6. The compound of claim 5 wherein the substituted benzyl group thereby forms a structure of Formula III.

7. The compound of claim 1 wherein the R⁵ substituent group is selected from the group consisting of an isobutyl group, a sec-butyl group, an amine group having the formula (CH₂)₄NH₂, and an amine group having the formula (CH₂)₄NHC(O)OCH₂Ph.

8. The compound of claim 1 wherein when the R⁶ substituent group and the R⁷ substituent group form a ring structure, the ring structure is a ring having from four to eight atoms wherein one of the atoms is a nitrogen.

9. The compound of claim 1 wherein the R⁷ substituent group is a hydroxymethyl group.

10. The compound of claim 1 wherein the R⁸ substituent group is selected from the group consisting of a methoxy group, a hydroxyl group, and a 1,4 diaminobutane group.

11. The compound of claim 1 wherein R⁸ is a peptide moiety comprising a first amino acid wherein the amino acid is selected from a group consisting of Arginine Lysine, and a variant of Arginine wherein an N-terminus of the first amino acid is bound at the R⁸ position and the additional substituent group extends from a C terminus of the first amino acid.

12. The compound of claim 11 wherein the first amino acid is Ornithine.

13. The compound of claim 11 wherein the first amino acid is an Ornithine having a BOC group extending from a side chain of the Ornithine.

14. The compound of claim 11 wherein the first amino acid is an Ornithine having a 2-pyrimidine group extending from a side chain of the Ornithine.

15. The compound of claim 11 wherein the additional substituent group extending from a C terminus of the first amino acid is selected from the group consisting of NH₂, NHC₄H₉, NH(CH₂)₃Ph, and N(CH₂)₅.

16. The compound of claim 1 wherein R⁸ is a peptide moiety comprised of two amino acids wherein a first amino acid is selected from a group consisting of Arginine, Lysine, and a variant of Arginine and a second amino acid is Proline wherein an N terminus of the first amino acid is bound at the R⁸ position, a C terminus of the first amino acid is bound to an N terminus of the second amino acid and an additional substituent group extends from the C terminus of the second amino acid.

17. The compound of claim 16 wherein the first amino acid is Ornithine.

18. The compound of claim 16 wherein the first amino acid is a BOC Ornithine.

19. The compound of claim 16 wherein the first amino acid is a 2-pyrimidine.

20. The compound of claim 16 wherein the additional substituent group extending from the C terminus of the second amino acid is selected from a group consisting of a primary amine and a secondary amine.

21. The compound of claim 16 wherein the additional substituent group extending from the first amino acid is selected from the group consisting of NH₂, NHC₄H₉, NH(CH₂)₃Ph, and N(CH₂)₅.

22. A compound of Formula III: 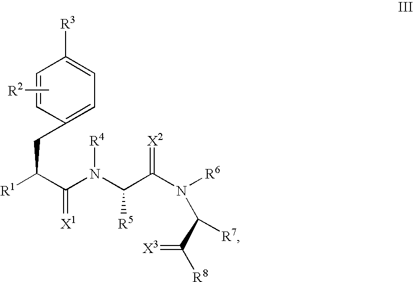
wherein
(a) X¹, X², X³ are identical or different and are independently selected from the group consisting of an oxygen atom, a sulfur atom and two hydrogen atoms;
(b) R¹ is a substituent group selected from the group consisting of a hydrogen, 1 to 16 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 10 carbon hydroxyalkyl group, a 1 to 6 carbon alkylamino group, a 1 to 6 carbon dialkylamino group, a 1 to 6 carbon aminoalkyl group, a 1 to 6 carbon alkoxyamino group, or a substituent group defined by Formula II(a): 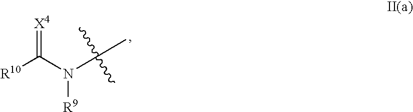
wherein R⁹ is a substituent group selected from the group consisting of a hydrogen atom and a 1 to 6 carbon alkyl; R¹⁰ is a substituent group selected from the group consisting of a hydroxyl group, a hydroxyalkyl, a 1 to 17 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 6 carbon alkylamino group and a 1 to 6 carbon dialkylamino group, and an aminoalkyl group; and X⁴ is a substituent group selected from the group consisting of an oxygen atom, a sulfur atom, and two hydrogen atoms;
(c) R² is a substituent group selected from a group consisting of a hydrogen atom, a 1 to 6 carbon alkyl group, a hydroxyl group, a 1 to 10 carbon alkoxy group, a halo group, an amine group, a 1 to 6 carbon alkylamino group, a 1 to 6 carbon dialkylamino group, a carboxylic acid ester, an amide group, a 1 to 2 ring aralkyloxy group, a substituent group forming a ring structure with R³, and a substituent group forming a ring structure with R⁴;
(d) R³ is a substituent group which is selected from the group consisting of a hydrogen atom, a hydroxyl group, a 1 to 6 carbon alkyl group, a 1 to 10 carbon alkoxy group, a 1 to 6 carbon hydroxyalkyl group, a 1 to 2 ring aralkyloxy group, a 1 to 2 ring aryloxy group, a halo group, a sulfidryl group, an alkylsulfidryl group, an amine group, a 1 to 6 carbon alkylamino group, a carboxylic acid ester, an amide group, a 1 to 6 carbon dialkylamino group, a substituent group forming a ring with R², a substituent group forming a ring with R⁴, and a substituent group represented by a structure of Formula II(b): 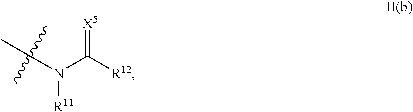
wherein X⁵ is a substituent group selected from the group consisting of an oxygen atom, a sulfur atom, and two hydrogen atoms a methylene group and a hydroxyethylene group; R¹¹ is a substituent group selected from the group consisting of a hydrogen atom and a 1 to 6 carbon alkyl group; and R¹² is a substituent group selected from the group consisting of a hydroxyl group, a 1 to 15 carbon alkyl group, a 1 to 10 carbon alkoxy group, a 1 to 2 ring aralkyloxy group, a 1 to 2 ring aryloxy group, a 1 to 6 carbon alkylamino group, and a 1 to 2 ring aralkylamino;
(e) R⁴ is a substituent group selected from the group consisting of a hydrogen atom, a 1 to 6 carbon alkyl group, a substituent group forming a ring with R³, and a substituent group forming a ring with R²;
(f) R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group;
(g) R⁶ is a substituent group selected from the group consisting of a hydrogen atom, a 1 to 6 carbon alkyl group, and a substituent group forming a ring with R⁷;
(h) R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group; and
(i) R⁸ is a substituent group which is selected from the group consisting of a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a secondary amine, a tertiary amine, a tertiary cyclic amine optionally substituted by a guanidinoalkyl group, a 1 to n carbon aminoalkylamino group, a 1 to n carbon aminoalkylamide group, a 1 to n carbon aminoalkyl-carbamate group, a 1 to n carbon aminoalkylurea group, a 1 to n carbon aminoalkylamidine group, a 1 to n carbon aminoalkylguanidine group, where n=2-6, and a peptide moiety comprised of 1-4 amino acids bound at the R⁸ position by an N-terminus end of the peptide moiety, wherein a plurality of additional substituent groups extend from an N terminus, a side chain and a C terminus of each amino acid of the peptide moiety and each of the plurality of additional substituent groups may be independently selected from the group consisting of a hydroxyl group, a heterocyclic aromatic compound, an alkyl ester group, a cycloalkyl group, an amino alkyl group, an aminoalkylamino group, a primary amine, a secondary amine, and a tertiary amine.

23. The compound of claim 22 wherein the R¹ substituent group is a hydrogen atom.

24. The compound of claim 22 wherein the R¹ substituent group is represented by the structure of Formula II (a): 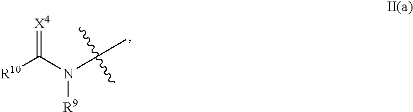
wherein X⁴ is an oxygen atom, R⁹ is a hydrogen atom and R¹⁰ is selected from the group consisting of a methyl group and a tert-butoxy group.

25. The compound of claim 22 wherein the substituent group of R² forms a ring in conjunction with a substituent group of R³ wherein the ring is a phenyl group.

26. The compound of claim 22 wherein the R³ substituent group is selected from the group consisting of a hydrogen atom, a hydroxyl group, a fluorine atom, and a benzoyl group.

27. The compound of claim 22 wherein the R³ substituent group is comprised of Formula IV: 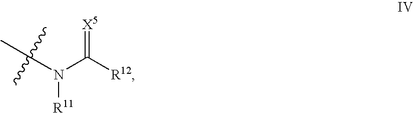
wherein X⁵ is an oxygen atom, R¹¹ is a hydrogen atom and R¹² is a benzoyl group.

28. The compound of claim 22 wherein the R⁵ substituent group is selected from the group consisting of an isobutyl group, a sec-butyl group, an amine group having the formula (CH₂)₄NH₂, and an amine group having the formula (CH₂)₄NHC(O)OCH₂Ph.

29. The compound of claim 22 wherein the R⁶ substituent group and the R⁷ substituent group form a ring structure wherein the ring structure is a ring having from four to eight atoms wherein one of the atoms is a nitrogen.

30. The compound of claim 22 wherein the R⁷ substituent group is a hydroxymethyl group.

31. The compound of claim 22 wherein the R⁸ substituent group is selected from the group consisting of a methoxy group, a hydroxyl group, a piperidinyl substituted by a guanidinylmethyl group, and a 1,4 diaminobutane group.

32. The compound of claim 22 wherein R⁸ is comprised of a peptide moiety comprised of a first amino acid wherein the amino acid is selected from a group consisting of Arginine, Lysine, and a variant of Arginine wherein an N-terminus of the first amino acid is bound at the R⁸ position and the additional substituent group extends from a C terminus of the first amino acid.

33. The compound of claim 32 wherein the first amino acid is Ornithine.

34. The compound of claim 32 wherein the first amino acid is an Ornithine having a BOC group extending from a side chain of the Ornithine.

35. The compound of claim 32 wherein the first amino acid is an Ornithine having a 2-pyrimidine group extending from a side chain of the Ornithine.

36. The compound of claim 32 wherein the additional substituent group extending from a C terminus of the first amino acid is selected from the group consisting of NH₂, NHC₄H₉, NH(CH₂)₃Ph, and N(CH₂)₅.

37. The compound of claim 32 wherein the first amino acid is arginine, and the additional substituent group extending from a C terminus of the first amino acid is selected from the group consisting of NH₂, OMe, and N(CH₂)₅.

38. The compound of claim 22 wherein R⁸ is a peptide moiety comprising two amino acids wherein a first amino acid is selected from a group consisting of Arginine, Lysine, and a variant of Arginine and a second amino acid is Proline wherein an N terminus of the first amino acid is bound at the R⁸ position, a C terminus of the first amino acid is bound to an N terminus of the second amino acid and an additional substituent group extends from the C terminus of the second amino acid.

39. The compound of claim 38 wherein the first amino acid is Ornithine.

40. The compound of claim 38 wherein the first amino acid is a BOC Ornithine.

41. The compound of claim 38 wherein the first amino acid is an Ornithine having a 2-pyrimidine group extending from a side chain of the Ornithine.

42. The compound of claim 38 wherein the additional substituent group extending from the C terminus of the second amino acid is selected from a group consisting of a primary amine and a secondary amine.

43. The compound of claim 38 wherein the additional substituent group extending from the first amino acid is selected from the group consisting of NH₂, NHC₄H₉, NH(CH₂)₃Ph, and N(CH₂)₅.

44. The compound of claim 1 selected from the group consisting of:

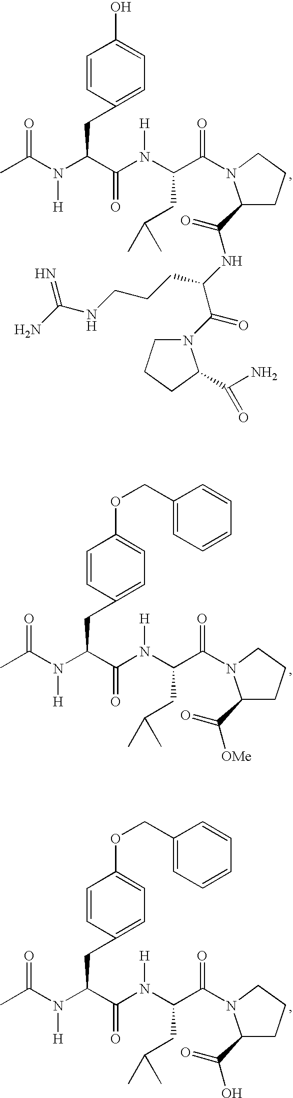

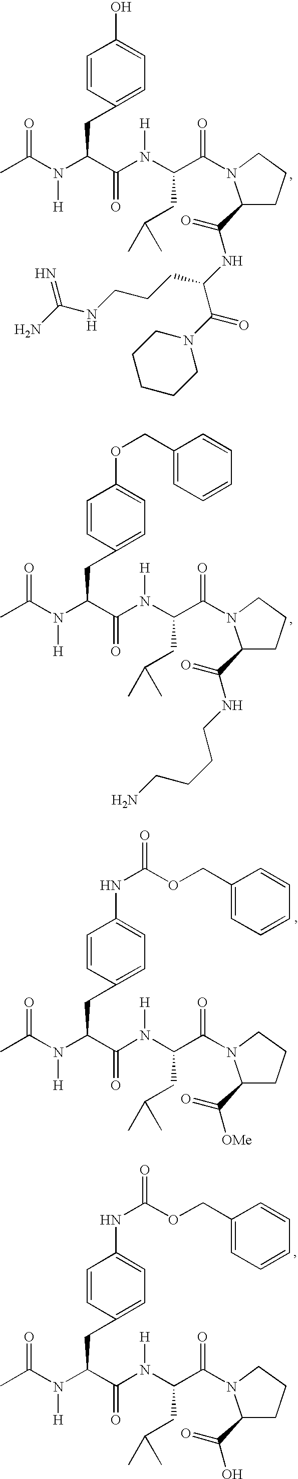

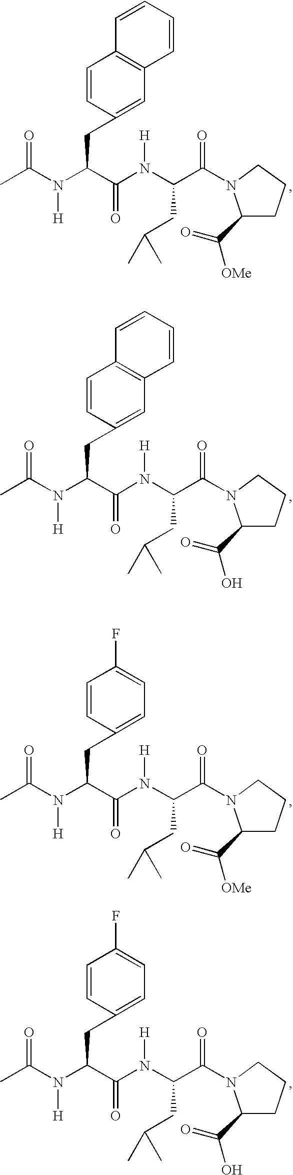

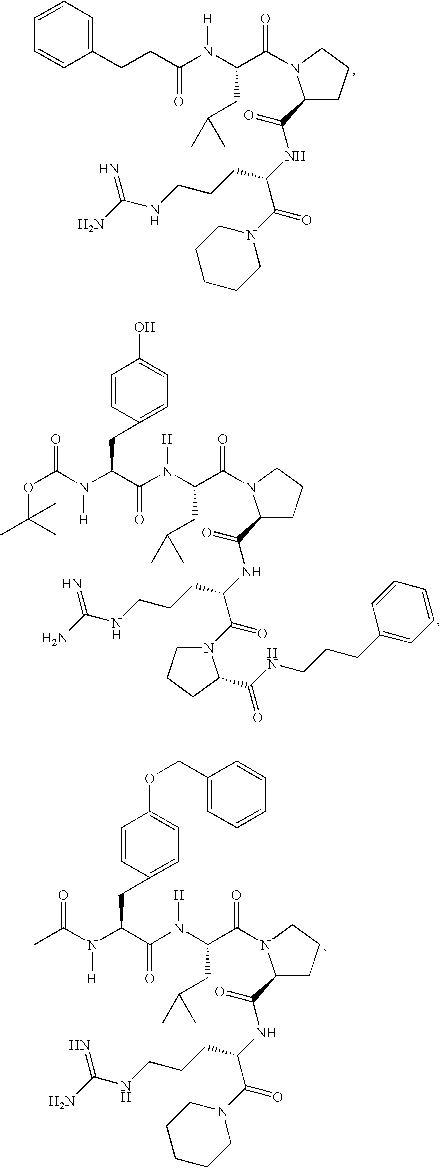

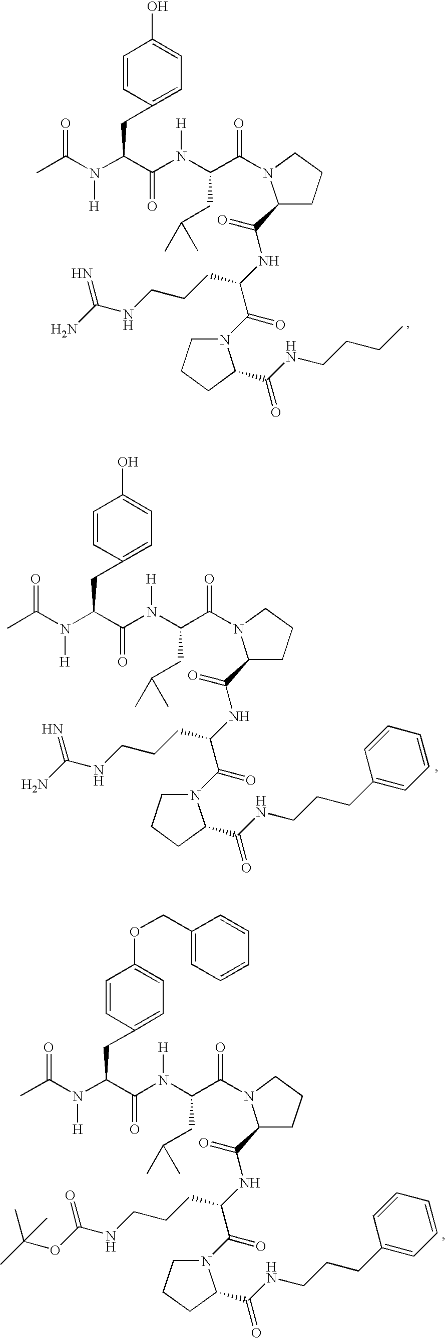

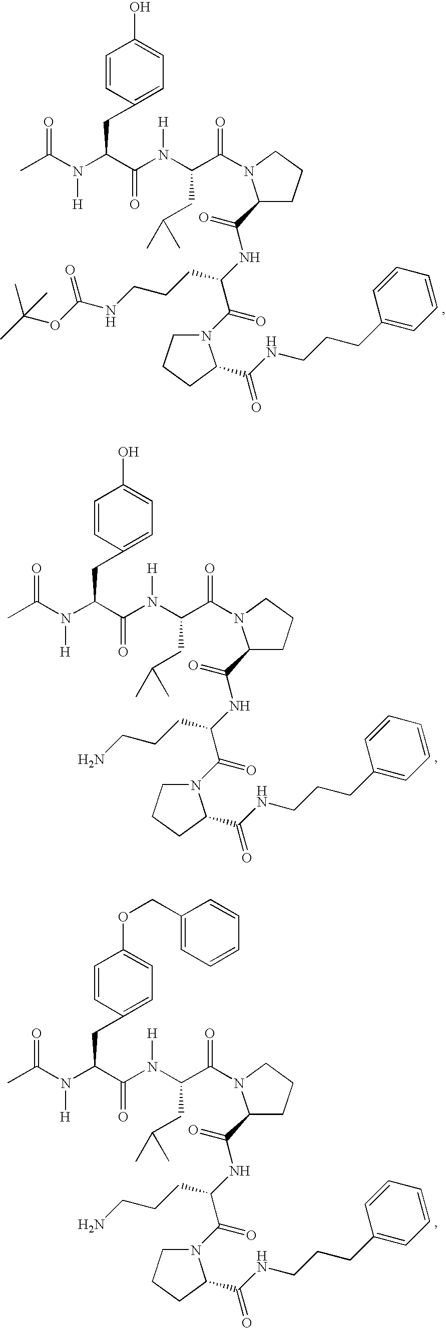

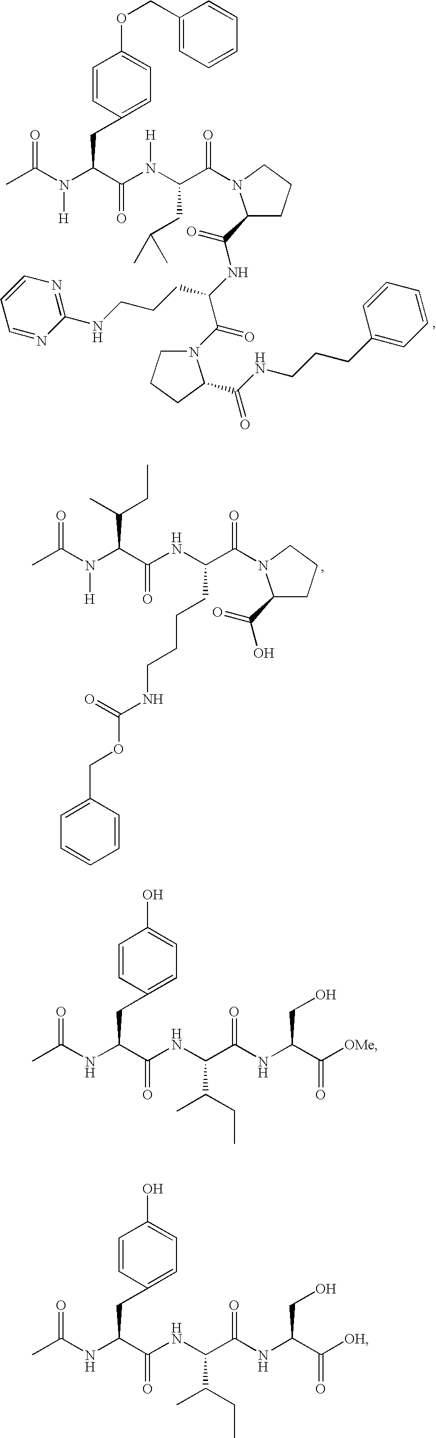

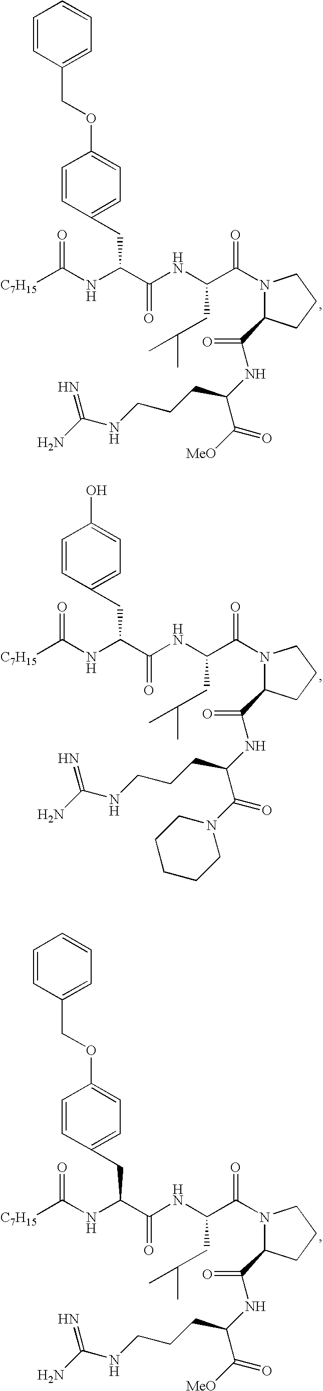

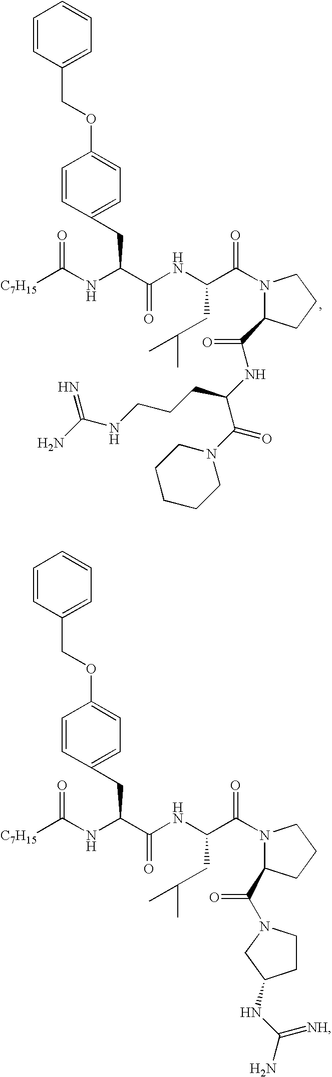

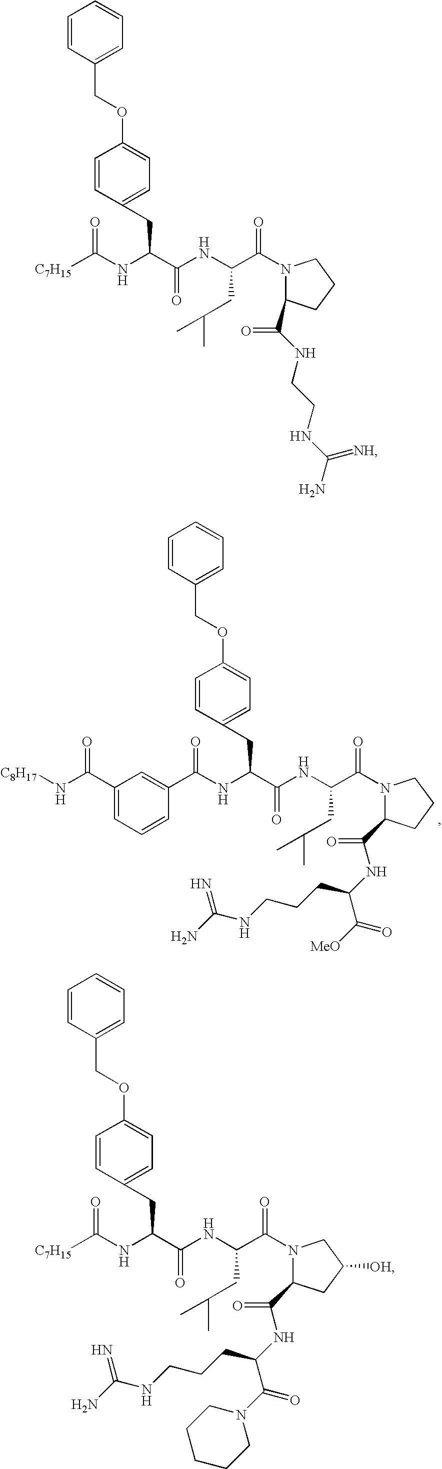

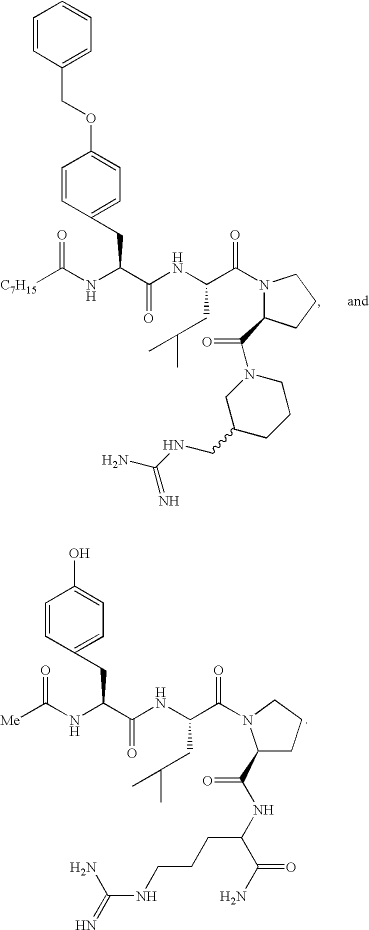

45. A method of treating a bacterial infection in a mammal comprising administering to the mammal having a bacterial infection a pharmaceutical composition comprising Formula I: 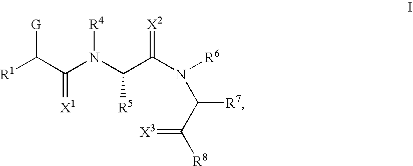
wherein
(a) X¹, X², X³ are identical or different and are independently selected from the group consisting of an oxygen atom, a sulfur atom and two hydrogen atoms;
(b) R¹ is a substituent group selected from the group consisting of a hydrogen, 1 to 16 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 10 carbon hydroxyalkyl group, a 1 to 6 carbon alkylamino group, a 1 to 6 carbon dialkylamino group, a 1 to 6 carbon aminoalkyl group and a 1 to 6 carbon alkoxyamino group, or a substituent group defined by Formula II(a): 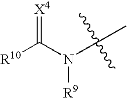
wherein R⁹ is a substituent group selected from the group consisting of a hydrogen atom and a 1 to 6 carbon alkyl; R¹⁰ is a substituent group selected from the group consisting of a hydroxyl group, a hydroxyalkyl, a 1 to 17 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 6 carbon alkylamino group and a 1 to 6 carbon dialkylamino group, and an aminoalkyl group; and X⁴ is a substituent group selected from the group consisting of an oxygen atom, a sulfur atom and two hydrogen atoms;
(c) G is a substituent group selected from the group consisting of a benzyl group, a substituted benzyl group, a phenyl group, a substituted phenyl group, a heterocyclic aromatic alkyl group, a 1 to 6 carbon alkyl group, and a substituted 1 to 6 carbon alkyl group;
(d) R⁴ is a substituent group selected from the group consisting of a hydrogen atom, a 1 to 6 carbon alkyl group, and a substituent group forming a ring with a G substituent group;
(e) R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group;
(f) R⁶ is a substituent group selected from the group consisting of a hydrogen atom, a 1 to 6 carbon alkyl group, and a substituent group forming a ring with R⁷;
(g) R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group; and
(i) R⁸ is a substituent group which is selected from the group consisting of a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a secondary amine, a tertiary amine, a tertiary cyclic amine optionally substituted by a guanidinylalkyl group, a 1 to n carbon aminoalkylamino group, a 1 to n carbon aminoalkylamide group, a 1 to n carbon aminoalkyl-carbamate group, a 1 to n carbon aminoalkylurea group, a 1 to n carbon aminoalkylamidine group, a 1 to n carbon aminoalkylguanidine group, where n=2-6, and a peptide moiety comprised of 1-4 amino acids bound at the R⁸ position by an N-terminus end of the peptide moiety, wherein an additional substituent group extends from an N terminus, a side chain and a C terminus of the peptide moiety wherein the additional substituent groups may be selected from the group consisting of a hydroxyl group, a heterocyclic aromatic compound, an alkyl ester group, a cycloalkyl group, an amino alkyl group, an aminoalkylamino group, a primary amine, a secondary amine, and a tertiary amine.

46. The method of claim 45 wherein the bacterial infection is caused by a Gram negative bacteria.

47. A composition for treating a bacterial infection in a mammal comprising a chemical compound of Formula I: 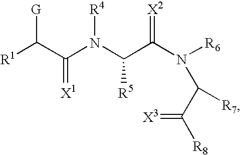
wherein
(a) X¹, X², X³ are identical or different and are independently selected from the group consisting of an oxygen atom, a sulfur atom and two hydrogen atoms;
(b) R¹ is a substituent group selected from the group consisting of a hydrogen, 1 to 16 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 10 carbon hydroxyalkyl group, a 1 to 6 carbon alkylamino group, a 1 to 6 carbon dialkylamino group, a 1 to 6 carbon aminoalkyl group, a 1 to 6 carbon alkoxyamino group, or a substituent group defined by Formula II(a): 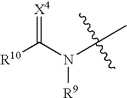
wherein R⁹ is a substituent group selected from the group consisting of a hydrogen atom and a 1 to 6 carbon alkyl; R¹⁰ is a substituent group selected from the group consisting of a hydroxyl group, a hydroxyalkyl, a 1 to 17 carbon alkyl group, a 3 to 6 carbon cycloalkyl group, a 1 to 2 ring aralkyl group, a 1 to 2 ring aryl group, a 1 to 2 ring heteroaryl group, a 1 to 10 carbon alkoxy group, a 1 to 6 carbon alkylamino group and a 1 to 6 carbon dialkylamino group, and an aminoalkyl group; and X⁴ is a substituent group selected from the group consisting of an oxygen atom, a sulfur atom, and two hydrogen atoms;
(c) G is a substituent group selected from the group consisting of a benzyl group, a substituted benzyl group, a phenyl group, a substituted phenyl group, a heterocyclic aromatic alkyl group, a 1 to 6 carbon alkyl group, and a substituted 1 to 6 carbon alkyl group;
(d) R⁴ is a substituent group selected from the group consisting of a hydrogen atom, a 1 to 6 carbon alkyl group, and a substituent group forming a ring with a G substituent group;
(e) R⁵ is a substituent group selected from the group consisting of a 1 to 6 carbon alkyl group, a 3 to 6 carbon cycloalkyl group, a 1 to 2 ring aralkyl group, a 1 to 6 carbon alkoxy group, a 1 to 6 carbon hydroxyalkyl, a 1 to 2 ring aralkoxyalkyl group, an aminoalkyl group, and derivatives thereof including derivatives of the aminoalkyl group wherein the aminoalkyl nitrogen atom has been derivatized to form an amide, a carbamate, an urea, and a guanidinium group;
(f) R⁶ is a substituent group selected from the group consisting of a hydrogen atom, a 1 to 6 carbon alkyl group, and a substituent group forming a ring with R⁷;
(g) R⁷ is a substituent group selected from the group consisting of a hydrogen atom, a 1 to 6 carbon alkyl group, a 1 to 2 ring aralkyl group, a 1 to 6 carbon hydroxyalkyl group, a 3 to 6 carbon cycloalkyl group, a 1 to 2 ring aralkyl group, and a 1 to 6 carbon hydroxyalkyl group wherein the R⁷ substituent group is capable of forming a cyclic ring structure or a substituted cyclic ring structure in conjunction with an R⁶ substituent group; and
(i) R⁸ is a substituent group which is selected from the group consisting of a hydroxyl group, a 1 to 17 carbon alkyl group, a 1 to 17 carbon alkoxy group, a 1 to 17 carbon hydroxyalkyl, a 1 to 2 ring aralkyloxy group, a 1 to 17 carbon alkylamino group, a 3 to 6 carbon cycloalkylamino group, a primary amine, a secondary amine, a tertiary amine, a tertiary cyclic amine optionally substituted by a guanidinylalkyl group, a 1 to n carbon aminoalkylamino group, a 1 to n carbon aminoalkylamide group, a 1 to n carbon aminoalkyl-carbamate group, a 1 to n carbon aminoalkylurea group, a 1 to n carbon aminoalkylamidine group, a 1 to n carbon aminoalkylguanidine group, where n=2-6, and a peptide moiety comprised of 1-4 amino acids bound at the R⁸ position by an N-terminus end of the peptide moiety where an additional substituent group extends from an N terminus, a side chain and a C terminus of the peptide moiety wherein the additional substituent groups may be selected from the group consisting of a hydroxyl group, a heterocyclic aromatic compound, an alkyl ester group, a cycloalkyl group, an amino alkyl group, an aminoalkylamino group, a primary amine, a secondary amine, and a tertiary amine.

48. The composition of claim 47 further comprising an active agent.

49. The composition of claim 48 wherein the active agent is an antibiotic.

50. The composition of claim 47 wherein the pharmaceutical composition is comprised of a therapeutically effective amount of the chemical compound represented by the structure of Formula I.

51. The composition of claim 47 wherein a route of administration of the pharmaceutical composition is selected from the group consisting of parenteral administration, oral administration.

52. The composition of claim 47 wherein the bacterial infection is caused by a Gram negative bacteria.

53. A method of making a compound selected from the group consisting of Formula I and Formula III, comprising the steps of:
(a) providing a solid phase synthesis support resin;
(b) coupling 2 to 5 amino acids to the solid phase support resin in the presence of a coupling agent to obtain a peptide wherein the amino acids may be selected from a group consisting of natural amino acids or derivatives thereof;
(c) adding a first substituent group to N terminus of the peptide;
(d) adding a second substituent group to a C terminus of the peptide;
(e) cleaving the peptide from the from the solid phase support resin resulting in a product selected from the group consisting of compounds having Formula I and Formula IV; and
(f) purifying the product.

54. A method of making a compound selected from the group consisting of Formula I and Formula III, comprising the steps of:
(a) providing a plurality of N-protected amino acids wherein each amino acid is protected at its N terminus by a protecting group wherein the plurality of amino acids may be selected from a group consisting of natural amino acids and derivatives thereof;
(b) coupling the plurality of N-protected amino acids so as to form a 2 to 5 amino acid peptide;
(c) removing the protecting groups from each amino acid in the peptide resulting in a product selected from the group consisting of compounds having Formula I and Formula III; and
(d) purifying the product.
